(19) United States
(12) Patent Application Publication
(10) Pub. No.: US 2022/0223227 A1
(43) Pub. Date: Jul. 14, 2022

(54) MACHINE LEARNING TECHNIQUES FOR IDENTIFYING MALIGNANT B- AND T-CELL POPULATIONS

(71) Applicant: BostonGene Corporation, Waltham, MA (US)

(72) Inventors: Olga Kudryashova, Moscow (RU); Mark Meerson, Moscow (RU); Vasiliy Minkov, Moscow (RU); Nikita Kotlov, Moscow (RU); Feliks Frenkel, Moscow (RU)

(73) Assignee: BostonGene Corporation, Waltham, MA (US)

(21) Appl. No.: 17/553,498

(22) Filed: Dec. 16, 2021

Related U.S. Application Data
(60) Provisional application No. 63/126,440, filed on Dec. 16, 2020.

(51) Int. Cl. G16B 30/00; G16B 20/00; G16B 20/20; G16B 40/20

(52) U.S. Cl. CPC G16B 30/00; G16B 40/20; G16B 20/20; G16B 20/00

(57) ABSTRACT

Techniques for identifying malignant cell populations. The techniques include: obtaining sequencing data previously obtained from a biological sample from a subject; processing the sequencing data to identify: a plurality of cell population estimates for a cell of a first type, the plurality of cell population estimates including a first cell population estimate and a second cell population estimate associated respectively with largest and second largest cell population estimates from among the identified plurality of cell population estimates; and features associated with the plurality of cell population estimates, the features including: a first feature indicative of a size of the first cell population estimate; and a second feature indicative of a ratio between sizes of the first cell population estimate and the second cell population estimate; and determining, using the features and a trained machine learning model, whether the first cell population estimate includes malignant cells of the first type.

### CROSS-REFERENCE TO RELATED APPLICATIONS

This application claims the benefit of priority under 35 U.S.C. 119(e) of U.S. provisional patent application No. 63/126,440, titled “MACHINE LEARNING TECHNIQUES FOR IDENTIFYING MALIGNANT B- AND T-CELL POPULATIONS”, filed on Dec. 16, 2020, which is incorporated by reference in its entirety herein.

### REFERENCE TO A SEQUENCE LISTING SUBMITTED AS A TEXT FILE VIA EFS-WEB RELATED APPLICATIONS

The present application contains a Sequence Listing which has been submitted in ASCII format via EFS-Web and is hereby incorporated by reference in its entirety. Said ASCII copy, created on Mar. 30, 2022, is named B146270020US01-SEQ-DGR and is 5,399 bytes in size.

### FIELD

Aspects of the technology described herein relate to machine learning techniques for analyzing sequencing data obtained from a biological sample obtained from a subject.

### BACKGROUND

The immune system utilizes a network of cells (e.g., B cells and T cells) to defend the body against harmful antigens. B cell receptors (BCRs) and T cell receptors (TCRs) comprise membrane receptor chains that recognize and bind these antigens. Specifically, B cell receptors comprise two immunoglobulin heavy chains (IgHs) and two immunoglobulin light chains of two types: lambda (IgL) and kappa (IgK). T cell receptors comprise alpha (TRA) and beta (TRB) chains or gamma (TRG) and delta (TRD) chains. Genetic mechanisms are used to vary regions of the receptor chains such that each unique B cell or T cell population recognizes a different, specific antigen, enabling the recognition of a wide assortment of antigens.

### SUMMARY

Some embodiments provide for a method, comprising: using at least one computer hardware processor to perform: obtaining sequencing data previously obtained from a biological sample from a subject; processing the sequencing data to identify: a plurality of cell population estimates for a cell of a first type, the plurality of cell population estimates including a first cell population estimate and a second cell population estimate associated respectively with largest and second largest cell population estimates from among the identified plurality of cell population estimates for the cell of the first type; and features associated with the plurality of cell population estimates, the features including: a first feature indicative of a size of the first cell population estimate; and a second feature indicative of a ratio between sizes of the first cell population estimate and the second cell population estimate; and determining, using the features and a trained machine learning model, whether the first cell population estimate includes malignant cells of the first type.

Some embodiments provide for a system, comprising: at least one computer hardware processor; and at least one non-transitory computer-readable storage medium storing processor-executable instructions that, when executed by the at least one computer hardware processor, cause the at least one computer hardware processor to perform: obtaining sequencing data previously obtained from a biological sample from a subject; processing the sequencing data to identify: a plurality of cell population estimates of a cell of a first type, the plurality of cell population estimates including a first cell population estimate and a second cell population estimate associated respectively with largest and second largest cell population estimates from among the identified plurality of cell population estimates for the cell of the first type; and features associated with the plurality of cell population estimates, the features including: a first feature indicative of a size of the first cell population estimate; and a second feature indicative of a ratio between sizes of the first cell population estimate and the second cell population estimate;

and determining, using the features and a trained machine learning model, whether the first cell population estimate includes malignant cells of the first type.

Some embodiments provide for at least one non-transitory computer-readable storage medium storing processor-executable instructions that, when executed by at least one computer hardware processor, cause the at least one computer hardware processor to perform: obtaining sequencing data previously obtained from a biological sample from a subject; processing the sequencing data to identify: a plurality of cell population estimates of a cell of a first type, the plurality of cell population estimates including a first cell population estimate and a second cell population estimate associated respectively with largest and second largest cell population estimates from among the identified plurality of cell population estimates for the cell of the first type; and features associated with the plurality of cell population estimates, the features including: a first feature indicative of a size of the first cell population estimate; and a second feature indicative of a ratio between sizes of the first cell population estimate and the second cell population estimate; and determining, using the features and a trained machine learning model, whether the first cell population estimate includes malignant cells of the first type.

In some embodiments, processing the sequencing data to identify the plurality of cell population estimates comprises: obtaining an initial estimate of cell populations; and generating the plurality of cell population estimates based on the initial estimate, wherein the initial estimate is different from the plurality of cell population estimates.

In some embodiments, the sequencing data comprises a plurality of sequence reads and obtaining the initial estimate of cell populations further comprises grouping sequence reads into groups based on similarity among sequence reads in the plurality of sequence reads.

In some embodiments, the initial estimate of cell populations comprises multiple initial cell population estimates; and obtaining the initial estimate of cell populations comprises obtaining, for each particular initial cell population estimate of at least some of the multiple initial cell population estimates: information indicative of a receptor chain associated with the particular initial cell population estimate; and sequence reads associated with the particular initial cell population estimate.

In some embodiments, at least some sequence reads of the sequence reads associated with the particular initial cell population correspond to identical complementarity determining regions (CDR3). In some embodiments, generating the plurality of cell population estimates further comprises clustering sequence reads associated with at least some of the multiple initial cell population estimates.

Some embodiments further comprise determining a size of each cell population estimate of the plurality of cell population estimates based on a number of sequence reads associated with each particular initial cell population estimate.

In some embodiments, the receptor chain includes an immunoglobulin heavy chain (IgH) or an immunoglobulin light chain, wherein the immunoglobulin light chain includes at least one of a kappa light chain (IgK) or a lambda light chain (IgL).

In some embodiments, the plurality of cell population estimates comprises a first set of cell population estimates generated for IgH, a second set of cell population estimates generated for IgK, and a third set of cell population estimates generated for IgL.

In some embodiments, the first set includes the first cell population estimate and the second cell population estimate, the second set includes a third cell population estimate and a fourth cell population estimate associated respectively with largest and second largest cell population estimates from among the second set, and the third set includes a fourth cell population estimate and a fifth cell population estimate associated respectively with largest and second largest cell population estimates from among the third set.

Some embodiments further comprise processing the sequencing data to identify: features associated with the second set of cell population estimates, the features including: a third feature indicative of a size of the third cell population estimate; and a fourth feature indicative of a ratio between sizes of the third cell population estimate and the fourth cell population estimate; and determining, using the features and the trained machine learning model, whether the third cell population estimate includes malignant cells of the first type.

Some embodiments further comprise processing the sequencing data to identify: features associated with the third set of cell population estimates, the features including: a fifth feature indicative of a size of the third cell population estimate; and a sixth feature indicative of a ratio between sizes of the fifth cell population estimate and the sixth cell population estimate; and determining, using the features and the trained machine learning model, whether the fifth cell population estimate includes malignant cells of the first type.

Some embodiments further comprise obtaining coverages of the second and third sets of cell population estimates; and determining, based on the coverages and the third and fifth features, whether to output a first result of determining whether the third cell population estimate includes malignant cells of the first type, a second result of determining whether the fifth cell population estimate includes malignant cells of the first type, or neither the first nor the second result.

In some embodiments, the sequencing data comprises RNA sequencing data.

In some embodiments, the sequencing data comprises raw DNA or RNA sequencing data, DNA exome sequencing data, DNA genome sequencing data, gene sequencing data, bias-corrected gene sequencing data, any sequencing data comprising data obtained from a sequencing platform, or any sequencing data derived from data obtained from a sequencing platform.

Some embodiments further comprise, prior to processing the sequencing data, filtering the sequencing data to exclude samples with a coverage below a specified coverage threshold, wherein the specified coverage threshold is between 0 and 200.

In some embodiments, the trained machine learning model was trained using RNA sequencing data obtained for biological samples from a plurality of subjects.

In some embodiments, the biological samples comprise non-tumor samples and tumor samples that are diagnosed with cancer. In some embodiments, the subject has, is suspected to have, or is at risk of having cancer.

In some embodiments, the trained machine learning model was trained using sequencing data previously obtained from biological samples comprising B cells.

In some embodiments, the trained machine learning model was trained using sequencing data previously obtained from biological samples comprising cells with associated receptor chains that include IgH.

In some embodiments, the trained machine learning model is one of a Naïve Bayes classifier, a support vector machine classifier, a random forest classifier, or an Adaboost classifier.

Some embodiments further comprise generating a graphical user interface (GUI) including a visualization indicating a result of processing the sequencing data, the visualization comprising a plurality of nodes including a first set of nodes, the first set of nodes representing a cell population estimate of the plurality of cell population estimates, wherein each node included in the first set of nodes represents a respective initial cell population estimate of the initial estimate of cell populations.

In some embodiments, the first set of nodes includes a first node representing a first initial cell population estimate of the initial estimate of cell populations and a second node representing a second initial cell population estimate of the initial estimate of cell populations, wherein the first node is connected to the second node by an edge.

In some embodiments, a visual characteristic associated with at least some of the nodes in the first set of nodes is indicative of a characteristic of the first cell population estimate.

In some embodiments, the visual characteristic associated with the at least some nodes in the first set of nodes comprises a respective size of each of the at least some nodes, a shading of each of the at least some nodes, and/or a color of each of the least some nodes.

In some embodiments, the sequencing data comprises at least 1 million sequence reads, at least 5 million sequence reads, at least 10 million sequence reads, at least 20 million sequence reads, at least 50 million sequence reads, or at least 100 million sequence reads.

In some embodiments, the sequencing data comprises bulk RNA sequencing (RNA-seq) data, single cell RNA sequencing (scRNA-seq) data, or next generation sequencing (NGS) data.

### BRIEF DESCRIPTION OF DRAWINGS

FIG. 1A is a diagram depicting a system for identifying a malignant cell population based on sequencing data, according to some embodiments of the technology described herein.

FIG. 1B is an example diagram illustrating generating estimate cell populations and identifying a malignant cell population using a machine learning model, according to some embodiments of the technology described herein.

FIG. 2 is a flowchart depicting a machine learning process 200 for identifying malignant cell populations using sequencing data obtained from a biological sample, in accordance with some embodiments of the technology described herein.

FIG. 3A is a diagram of an illustrative technique for generating cell population estimates from sequencing data obtained from a biological sample, in accordance with some embodiments of the technology described herein.

FIG. 3B depicts an illustrative example for generating cell population estimates from sequencing data obtained from a biological sample, in accordance with some embodiments of the technology described herein.

FIG. 4 is a flowchart depicting a process 400 for selecting a prediction associated with an immunoglobulin light chain, in accordance with some embodiments of the technology described herein.

FIG. 5 is a flowchart illustrating an example of process 500 for selecting a prediction associated with an immunoglobulin light chain, according to some embodiments of the technology described herein.

FIG. 6 is an example flowchart of process 600 for selecting a prediction associated with an immunoglobulin light chain, in accordance with some embodiments of the technology described herein.

FIGS. 7A-E are illustrative examples of the process 600 for selecting the prediction associated with an immunoglobulin light chain, in accordance with some embodiments of the technology described herein.

FIG. 8 is a flowchart of an illustrative process 800 for training a machine learning model to identify malignant cell populations in biological samples, in accordance with some embodiments of the technology described herein.

FIG. 9A is a plot summarizing the type of tumor associated with datasets used to train and test the example machine learning model, in accordance with some embodiments of the technology described herein. FIG. 9B is a plot indicating the IgH clonality distribution for example datasets, according to some embodiments of the technology described herein.

FIGS. 9C-E are plots indicating the size of the largest estimate cell population in each sample included in each example dataset, in accordance with some embodiments of the technology described herein. FIG. 9F-H are plots showing the proportion of the largest estimate cell population in a sample compared to the number of estimate cell populations identified for the sample, according to some embodiments of the technology described herein.

FIG. 10A is a screenshot of an example report indicating information about estimate and malignant cell populations identified for a biological sample, in accordance with some embodiments of the technology described herein.

FIG. 10B is an example graph showing cell population estimates when there is a prominent initial cell population estimate, in accordance with some embodiments of the technology describe herein.

FIG. 10C is an example map showing cell populations estimates when there is no prominent initial cell population estimate, in accordance with some embodiments of the technology describe herein.

FIG. 10D is a screenshot of an example report illustrating a set of cell population estimates associated with IgH, in accordance with some embodiments of the technology described herein.

FIG. 10E is a screenshot of an example report illustrating a set of cell population estimates associated with IgL, in accordance with some embodiments of the technology described herein.

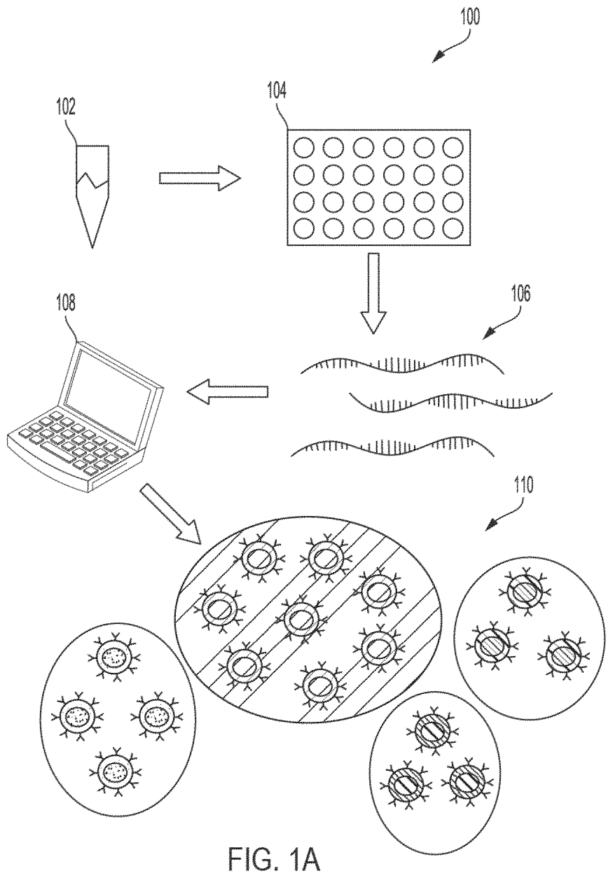

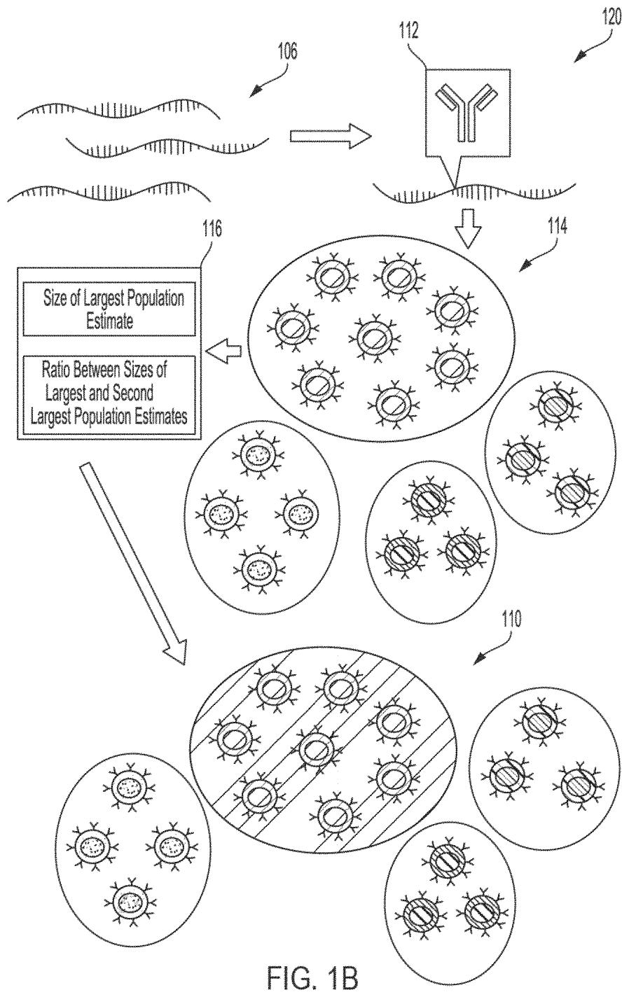

FIG. 1OF is a screenshot of an example report illustrating a set of cell population estimates associated with IgK, in accordance with some embodiments of the technology described herein.

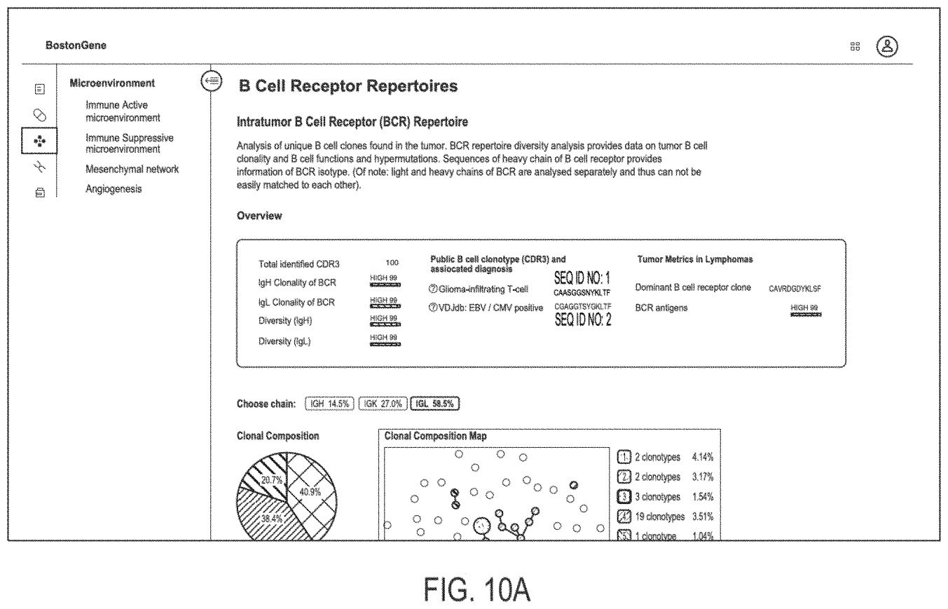

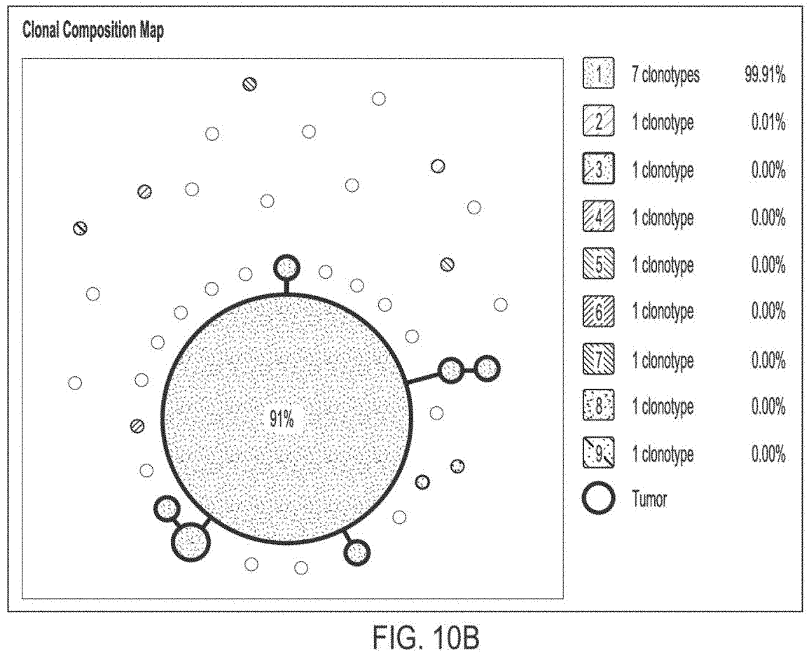

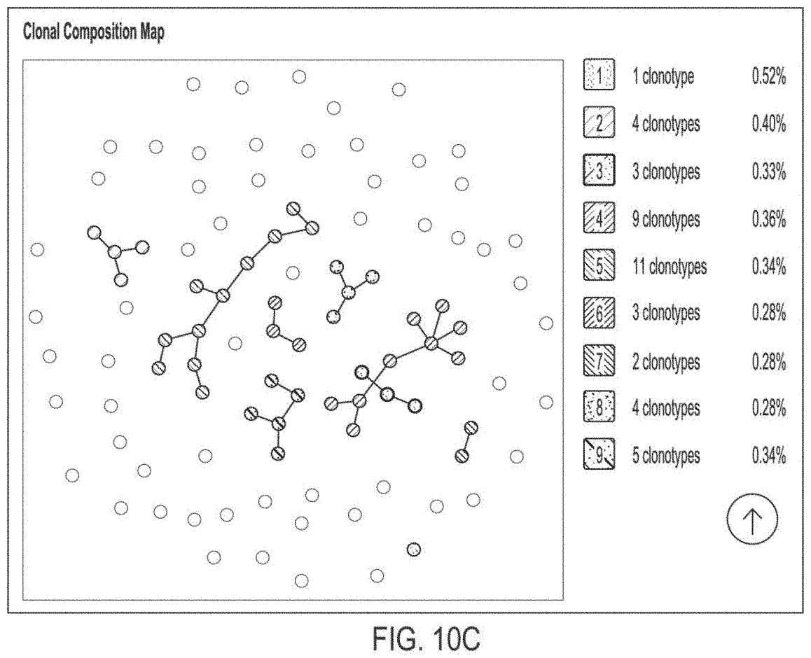

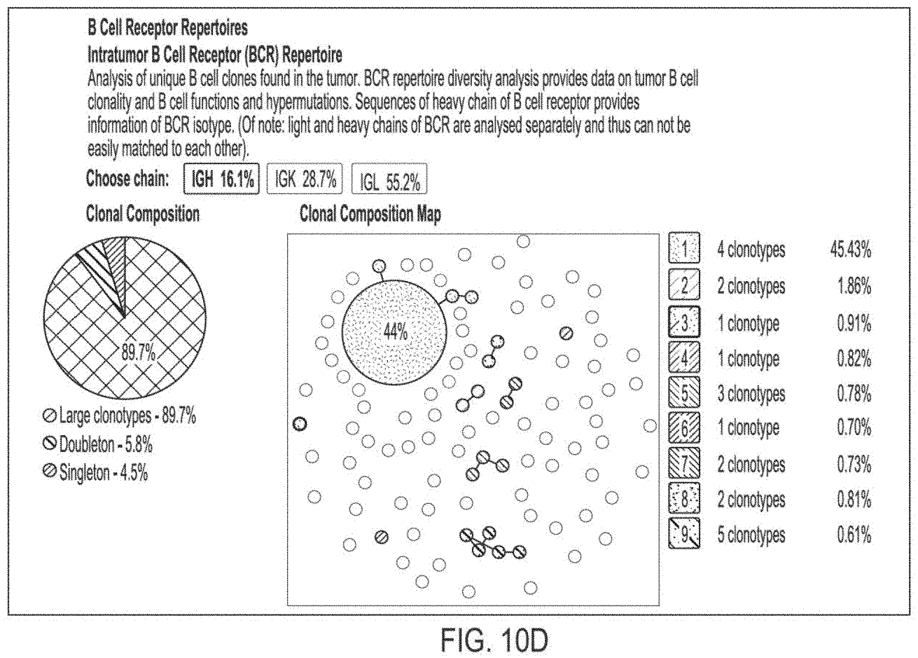

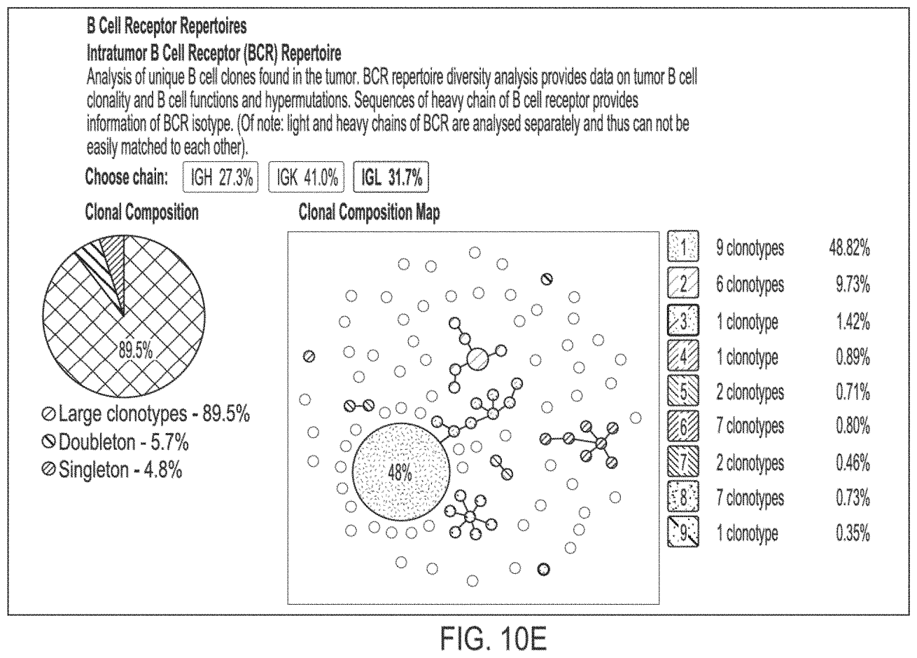

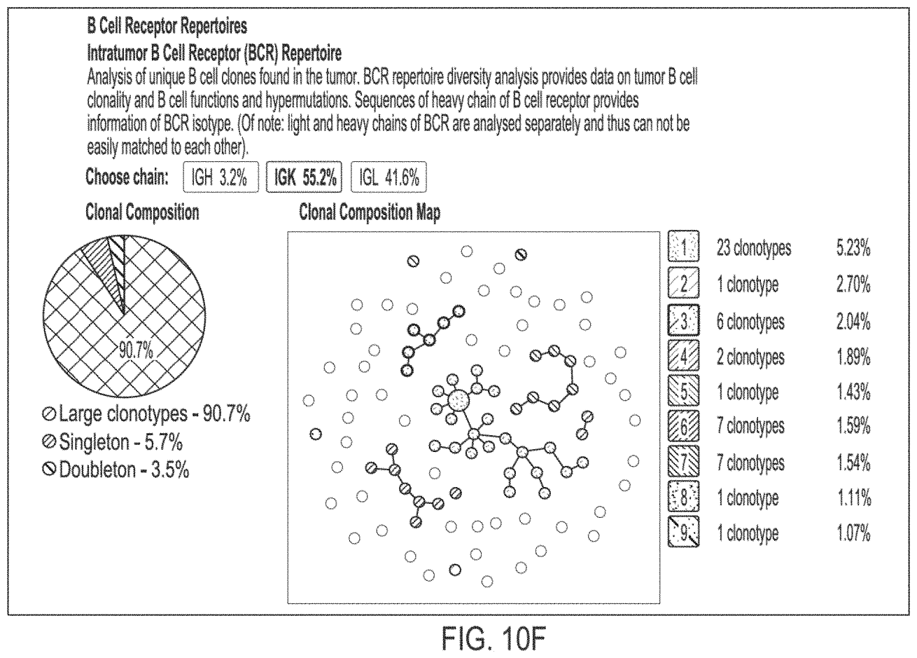

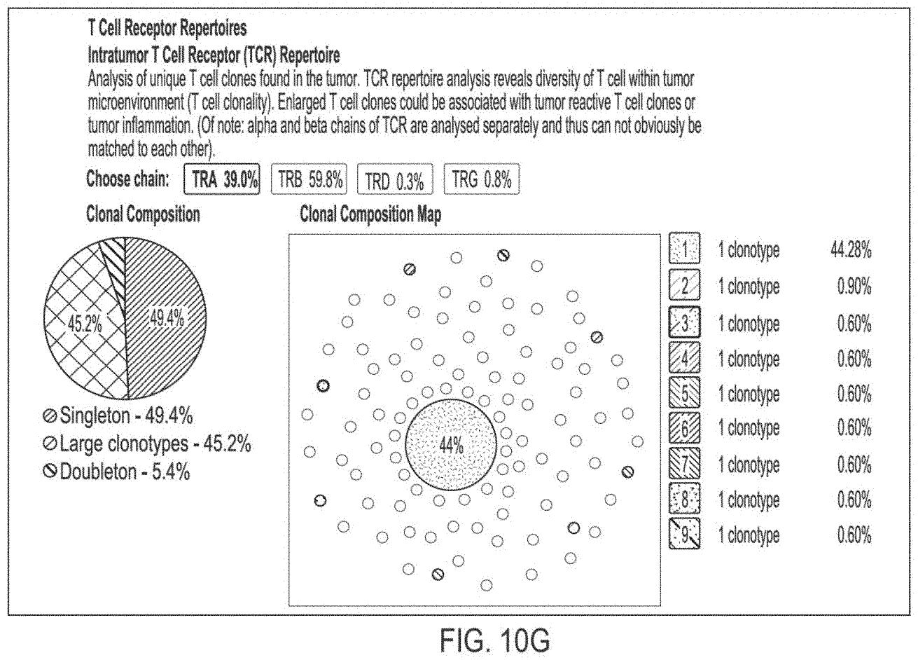

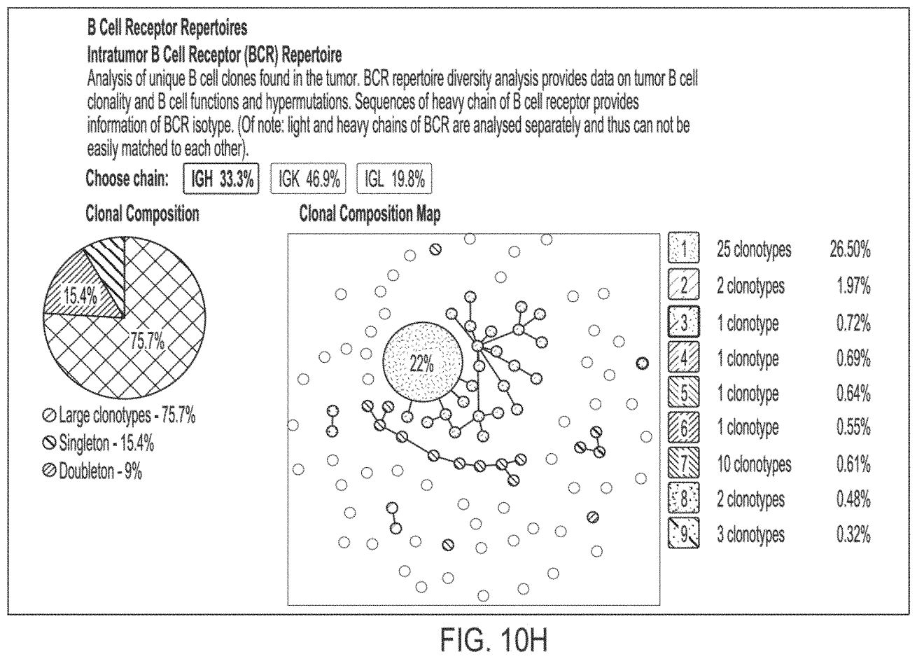

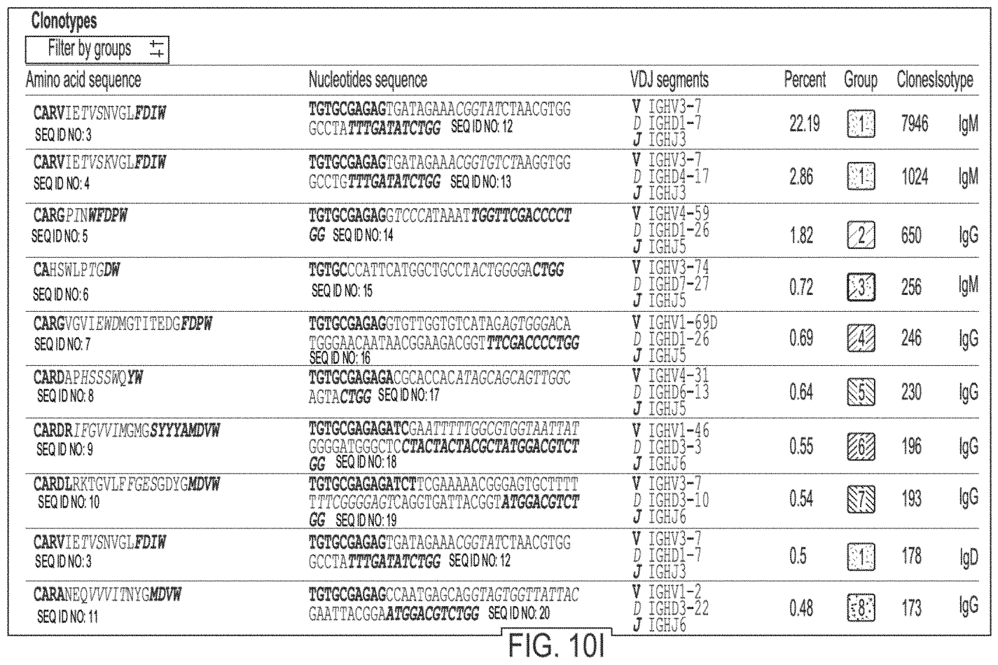

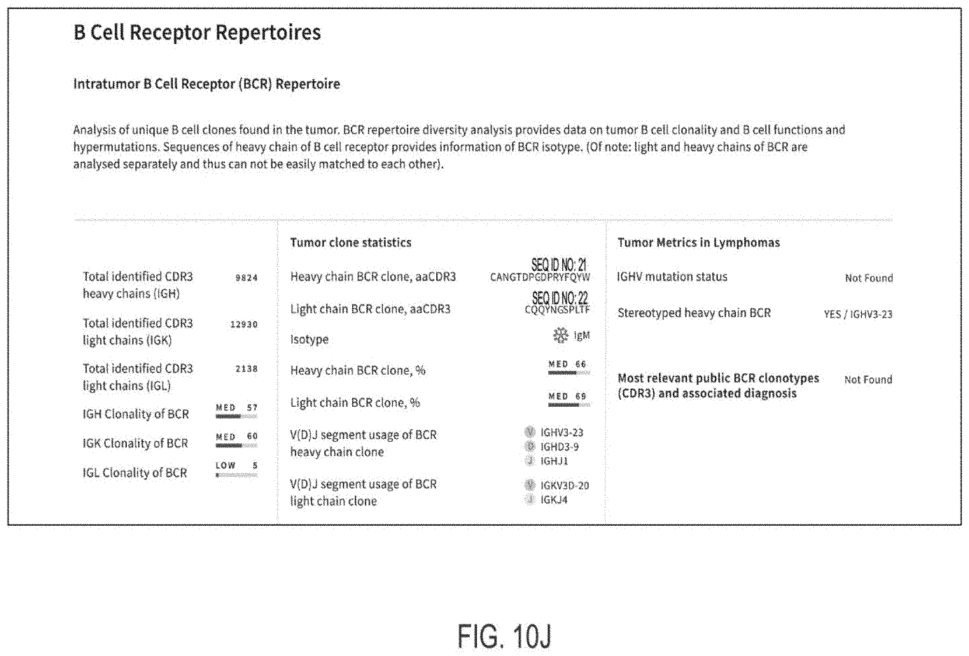

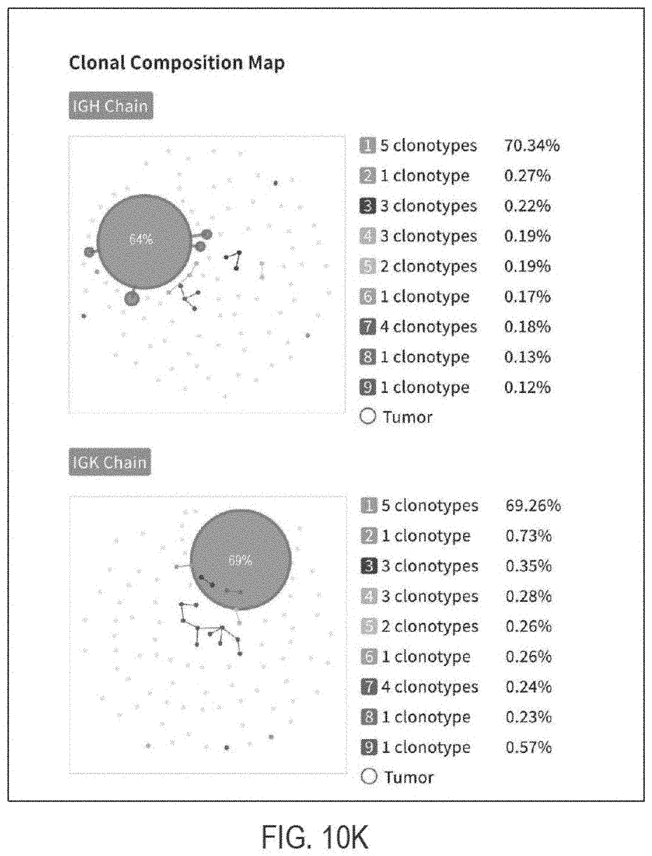

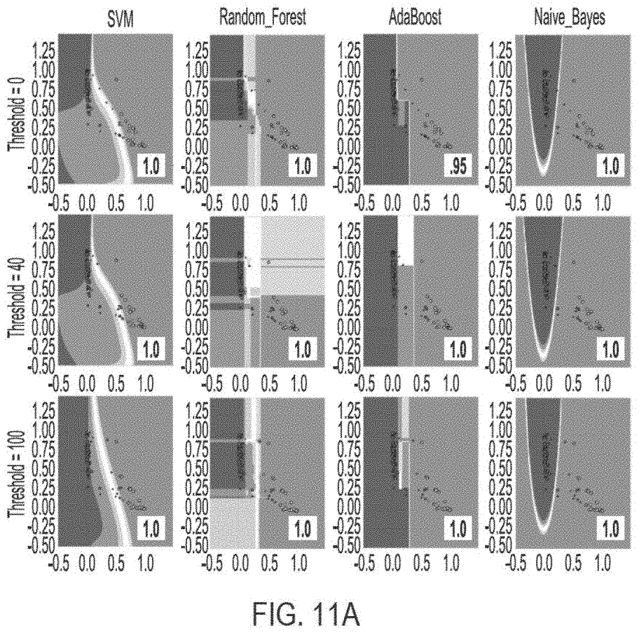

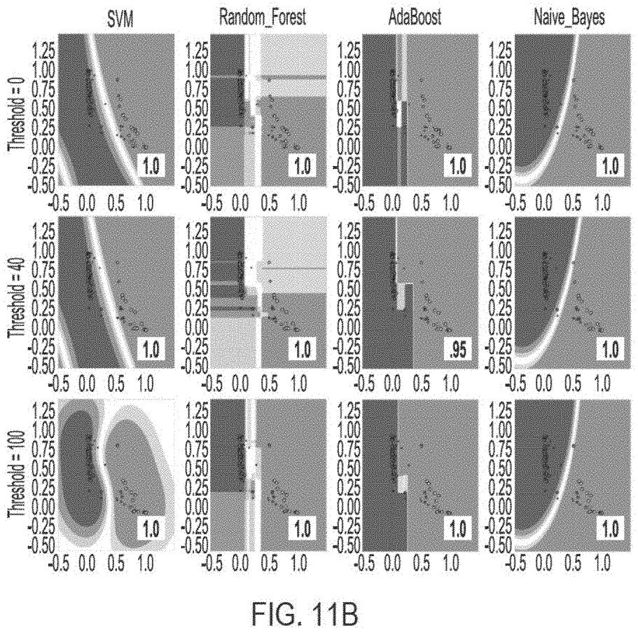

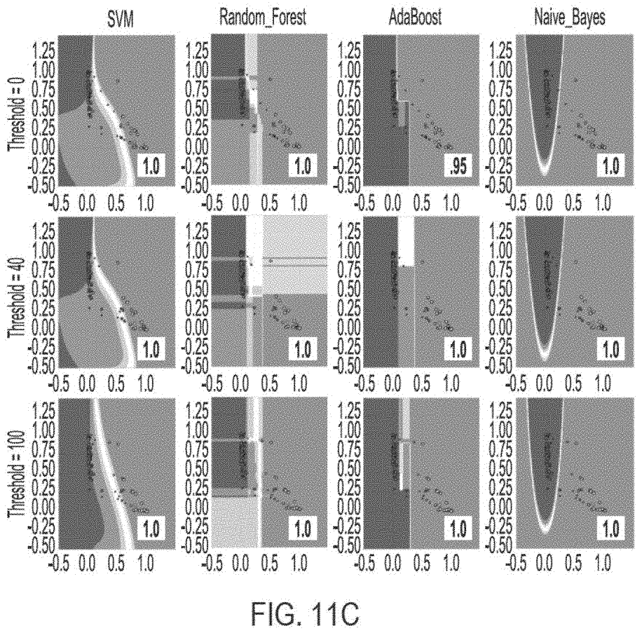

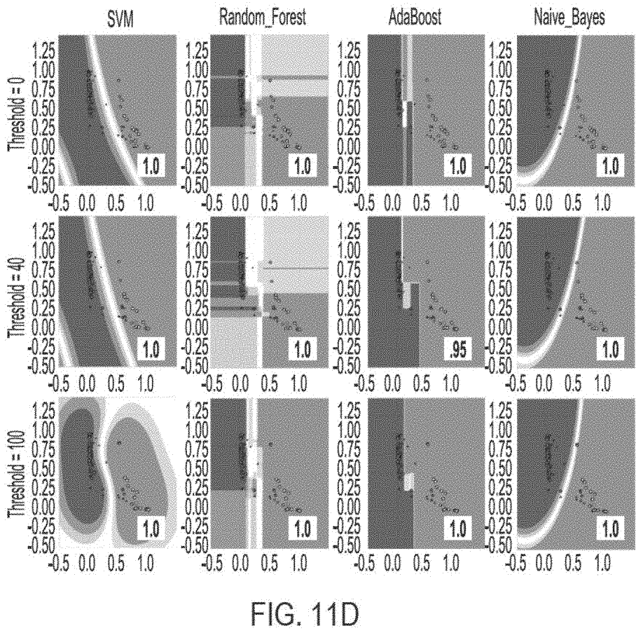

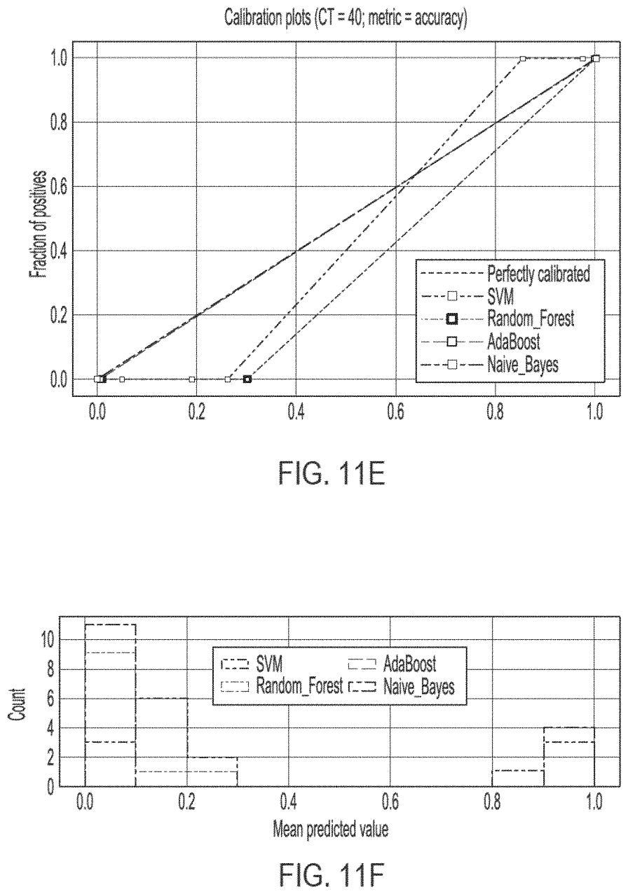

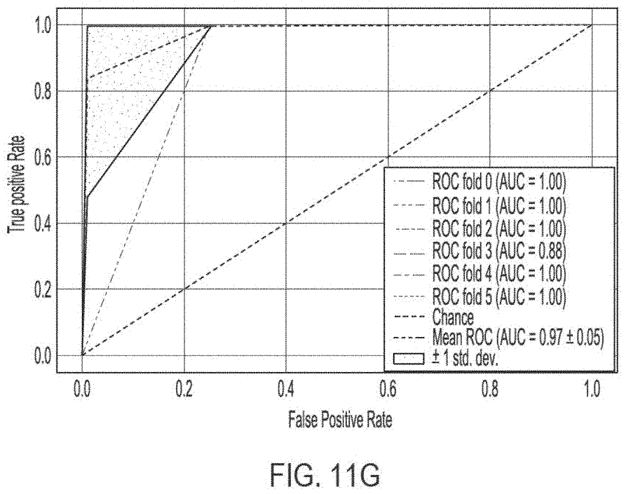

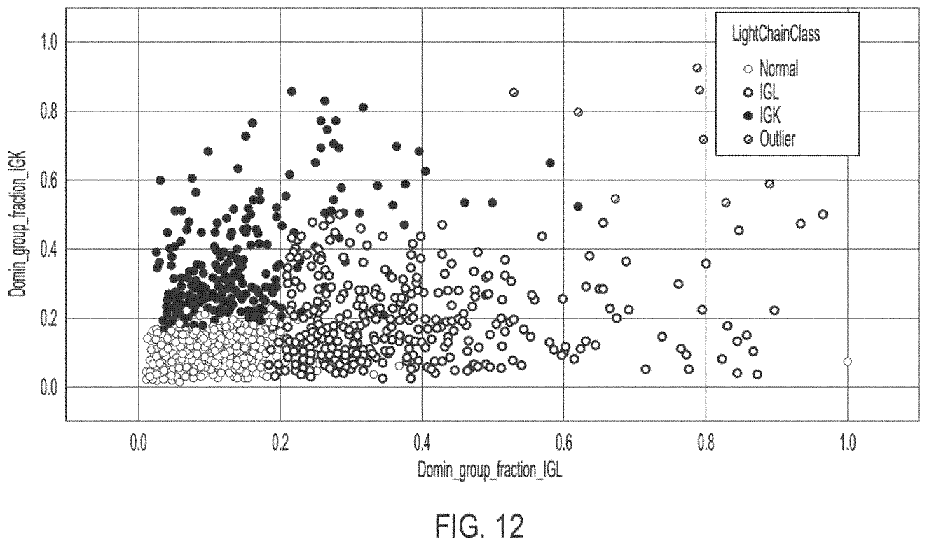

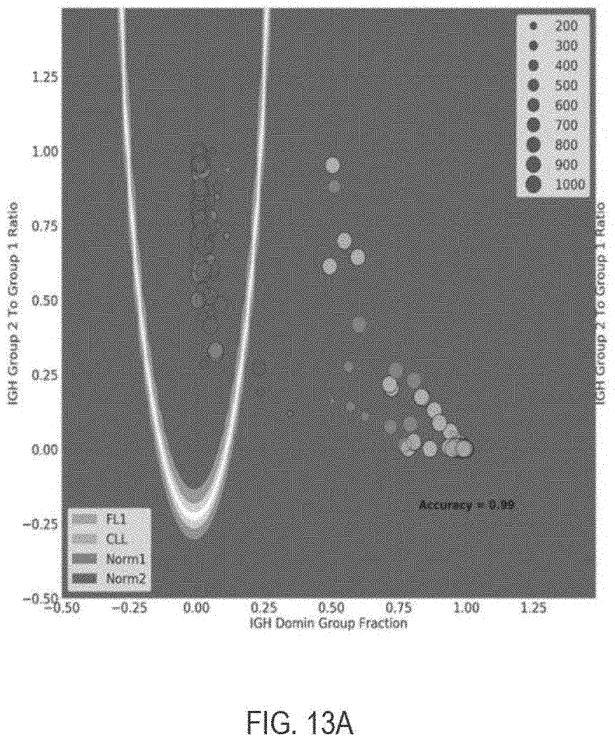

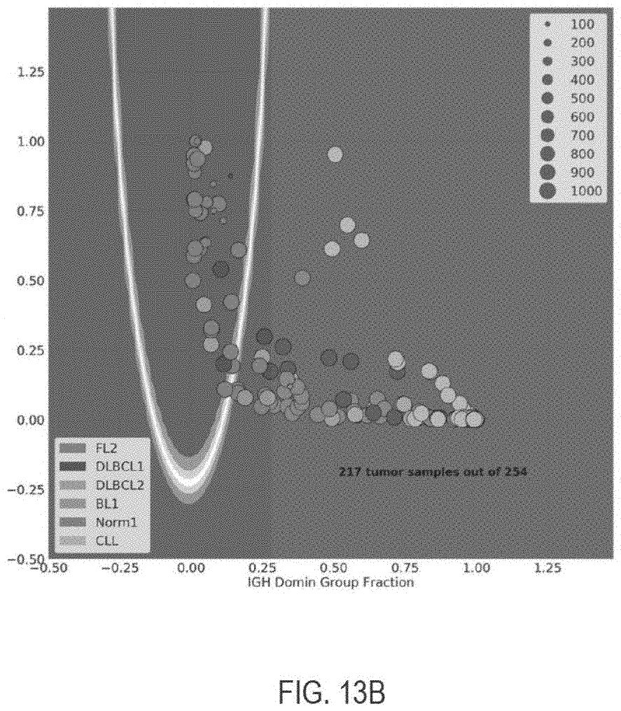

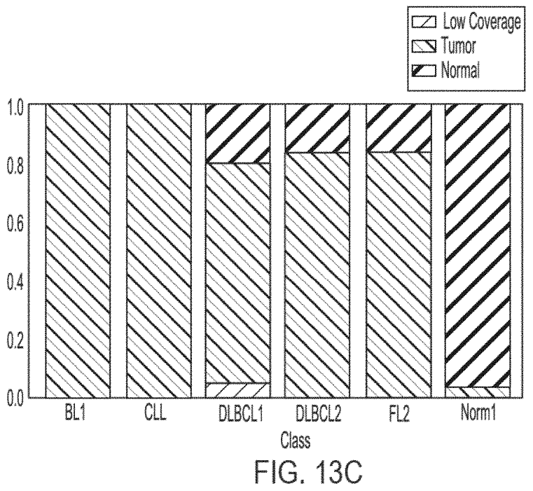

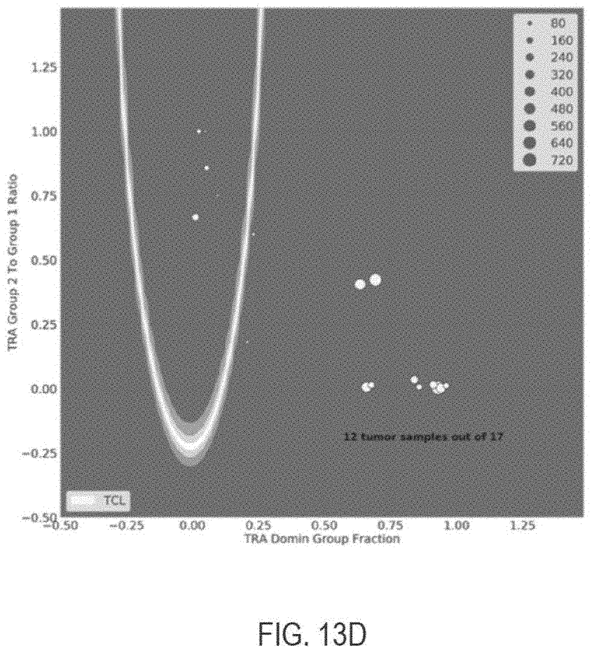

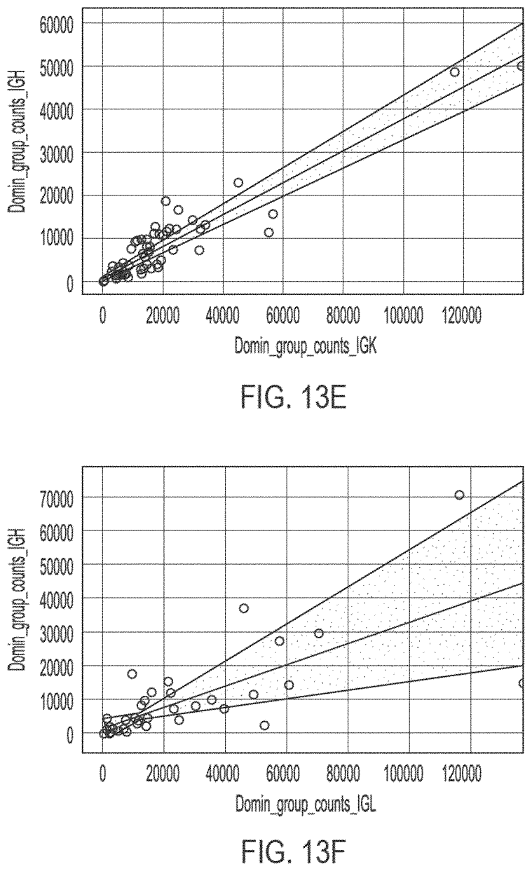

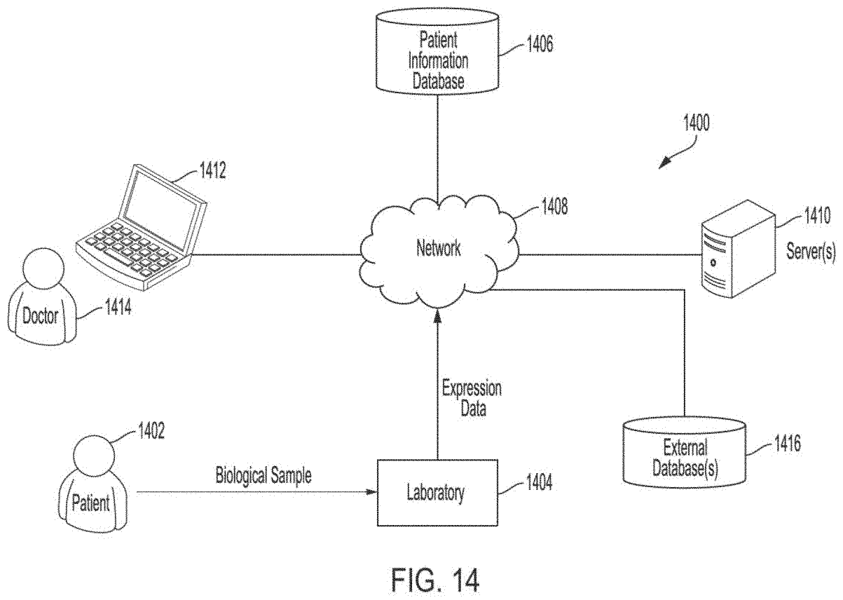

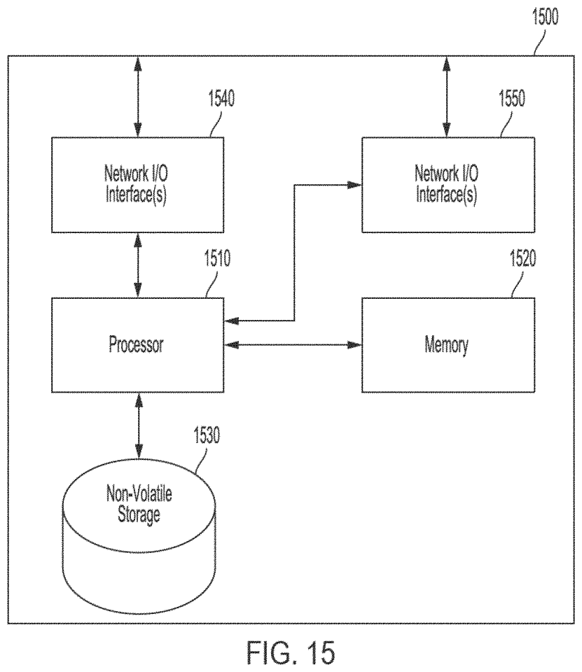

FIG. 10G is a screenshot of an example report illustrating a set of cell population estimates associated with TRA, in accordance with some embodiments of the technology described herein.

FIG. 10H is a screenshot of an example report illustrating a set of cell population estimates associated with IgH, in accordance with some embodiments of the technology described herein.

FIG. 10I is an example report that includes information about initial cell population estimates, in accordance with some embodiments described herein.

FIG. 10J is a screenshot of an example report indicating information about cell population estimates for a biological sample, in accordance with some embodiments of the technology described herein.

FIG. 10K is a screenshot of an example report illustrating sets of cell population estimates associated with IgH and IgK, in accordance with some embodiments of the technology described herein.

FIGS. 11A-D depict graphs illustrating the decision boundaries of different example machine learning models when selected by different parameters, in accordance with some embodiments of the technology described herein.

FIG. 11E is an example calibration plot for different example machine learning models, in accordance with some embodiments of the technology described herein.

FIG. 11F is an example probability distribution for different example machine learning models, in accordance with some embodiments of the technology described herein.

FIG. 11G is an example receiver operating characteristic (ROC) curve for a selected machine learning model, in accordance with some embodiments of the technology described herein.

FIG. 12 is a graph depicting the relationship between the size of the largest cell population and the immunoglobulin light chain selected for further analysis, in accordance with some embodiments of the technology described herein.

FIG. 13A is a graph depicting the predictions of a Naïve Bayes classifier used to process cell population estimates estimated based on BCR sequencing data, in accordance with some embodiments of the technology described herein.

FIG. 13B is a graph depicting the predictions of the Naïve Bayes classifier used to process cell population estimates estimated based on BCR sequencing data, in accordance with some embodiments of the technology described herein.

FIG. 13C is a chart illustrating example classification results using the Naïve Bayes classifier, in accordance with some embodiments of the technology described herein.

FIG. 13D is a graph depicting the predictions of a Naïve Bayes classifier used to process cell population estimates estimated based on TCR sequencing data, in accordance with some embodiments of the technology described herein.

FIG. 13E is a graph depicting the predictions of a Naïve Bayes classifier using process cell population estimates test datasets for B cell samples, in accordance with some embodiments of the technology described herein.

FIG. 13F is a graph depicting the number of IgH sequence reads associated with the largest estimate cell population compared to the number of IgL sequence reads associated with the largest estimate cell population, in accordance with some embodiments of the technology described herein.

FIG. 14 is a block diagram of an illustrative environment in which one or more embodiments of the technology described herein may be implemented.

FIG. 15 depicts an illustrative implementation of a computer system that may be used in connection with some embodiments of the technology described herein.

### DETAILED DESCRIPTION

Aspects of the disclosure relate to machine learning techniques for analyzing sequencing data from a biological sample obtained from a subject that may have been diagnosed with or is at risk of having cancer or an immune-related disease and identifying one or more malignant cell populations for the biological sample based on a result of the machine learning analysis. The techniques involve processing the sequencing data to identify cell population estimates for the biological sample and determining, using features of the cell population estimates and a trained machine learning model, whether a cell population estimate includes malignant cells of a particular type. The techniques for determining whether a cell population estimate includes malignant cells are useful for numerous applications including, but not limited to, detecting and treating a tumor at various stages of development, facilitating identification of one or more therapeutically effective treatments for the subject (which can subsequently be administered), and dynamically analyzing tumor progression over time.

As described above, one important application of the techniques developed by the inventors and described herein is analyzing sequencing data from a biological sample obtained from a subject that may have been diagnosed with or is at risk of having cancer or an immune-related disease. Cancers and immune-related diseases develop when harmful genetic changes (e.g., mutations) occur within the genes of a cell. When such mutations occur, the cell may lose its ability to recognize when to stop replicating, causing uncontrollable growth of the cell population originating from the damaged cell (e.g., tumor growth). For example, immune diseases or cancers specific to the immune system develop when harmful genetic changes occur within lymphocytes (e.g., B cells and T cells), leading to uncontrolled growth of lymphocyte populations.

There are typically many different lymphocyte populations in the immune system, each defined by unique regions of their receptors (e.g., B cell receptors and T cell receptors) that enable the recognition of specific antigens. Generally, in a healthy state, there is no dominant lymphocyte population. However, if a mutation occurs, it may cause one or more populations to grow uncontrollably and become larger than the others.

The treatment of cancers and immune-related diseases involves targeting malignant cell populations, from among many healthy cell populations, to halt tumor growth and eliminate the possibility of further uncontrolled replication. To do so, it is important to evaluate the molecular properties of the malignant cell populations. For example, personalized medicine utilizes neoantigens (e.g., antigens expressed specifically by the tumor) of malignant cell populations to develop immunotherapies that specifically target the diseased tissue. The inventors have therefore recognized the importance of accurately identifying a malignant cell population from among multiple various cell populations in diseased tissue.

The inventors have recognized that conventional methods for identifying malignant cell populations have multiple drawbacks and may be improved upon. Such conventional methods typically involve: (1) defining cell populations as those having receptor chains (e.g., B cell receptor chains and T cell receptor chains) with identical complementarity determining regions (CDR3); and (2) identifying the largest cell population as the malignant cell population.

One problem with such conventional techniques, and recognized by the inventors, is the manner in which a cell population is defined. In certain cell populations (e.g., B cell populations), environmental factors cause genetic changes in the CDR3 region of the cell receptor chain. Therefore, the CDR3 region can differ among cells belonging to the same cell population. By defining a cell population as only including cells having receptor chains with identical CDR3 regions, conventional techniques exclude those cells that have undergone genetic changes, but which otherwise belong to the cell population. Therefore, the use of such conventional techniques can lead to inaccurate cell population estimates that affect the ability to correctly identify the malignant cell population. For example, such techniques may underestimate the size of the largest cell population in a biological sample, making it indistinguishable from other cell populations.

Another problem with the conventional techniques for identifying malignant cell populations, and recognized by the inventors, is that it is not always possible to identify a cell population as malignant based on population size alone. While the malignant cell population is typically the largest cell population in an advanced stage of a disease, where there has been uncontrolled tumor growth over a long period of time, this is not always the case. For example, in early stages of a disease, where the malignant cell population is just beginning to develop, this population may be indistinguishable from other cell populations. However, the inventors have appreciated that it is desirable to identify a tumor in its beginning stages in order to prevent further spread that would result in greater harm to the patient. As another example, in minimal residual disease, a very small malignant cell population remains after treatment, which cannot be identified using techniques that rely on identifying the largest cell population in the biological sample.

The inventors have developed techniques for more accurately identifying malignant cell populations (e.g., B cells and T cells) in a biological sample for a subject that address the above-described problems of conventional identification techniques. First, the inventors have developed techniques for generating cell population estimates that account for the genetic changes that occur in the receptor chains of cells. In some embodiments, the techniques include using sequencing data corresponding to the receptor chains of cells to identify initial cell population estimates. For example, this includes grouping sequence reads that are associated with (e.g., align to) identical regions of a receptor chain (e.g., the CDR3 region of the receptor chain) to identify an initial estimate of cell populations. In some embodiments, the techniques include generating the cell population estimates based on the initial estimate of cell populations. For example, clustering techniques may be applied to sequencing data associated with the cell populations in the initial estimate of cell populations to generate the final cell population estimates. The clustering techniques may be used to combine initial cell population estimates having similar sequencing data (e.g., fewer than a threshold number of differences) to generate the resulting cell population estimates. Accordingly, the techniques account for slight genetic differences between cells that belong to the same cell population, leading to more accurate cell population estimates.

The inventors have also developed machine learning techniques to identify malignant cell populations based on multiple features derived from sequencing data (e.g., data obtained by processing the biological sample with a sequencing technique, such as next-generation sequencing (NGS)). In some embodiments, the features are indicative of the size of the largest cell population within the sample and a ratio between sizes of the largest and second largest cell populations within that sample. The features are processed using a trained machine learning model to determine whether the largest cell population includes malignant cells. By using multiple features, the machine learning techniques described herein can identify malignant cell populations more accurately than conventional techniques that rely on population size alone.

The techniques developed by the inventors and described herein address the above shortcomings of conventional methods for identifying a malignant cell population and for identifying effective therapies for treating such a malignant cell population.

First, the techniques described herein generate estimate cell populations using a multi-stage approach including generating an initial estimate of cell populations and generating the estimate cell populations based on the initial estimate of cell populations. As a result, in cases where a biological cell population includes cells that have undergone genetic mutation, it may nonetheless be possible to accurately include those cells in the estimate cell population. Not only does this allow for the accurate estimation of the size of the cell population, but it also provides information about the diversity of cells included within the same cell population. Such information may be useful for identifying a treatment that can effectively treat all cells in the cell population.

Moreover, the techniques described herein identify a malignant cell population based on multiple features derived from the cell population estimate. For example, such features not only include the size of the largest cell population estimate, but also the ratio between the sizes of the largest and second largest cell population estimates. By processing multiple features using a trained machine learning model, the techniques can more accurately identify malignant cell populations, even when the cell populations seem to be indistinguishable in size.

Furthermore, by using the techniques for generating accurate cell population estimates, in combination with the machine learning techniques, it is possible to develop a comprehensive understanding of a tumor, monitor its progression over time, and identify effective treatments for the subject. As described above, a cell population estimate provides information about the diversity of its cells (e.g., cells having diverse sequencing data). When classified as malignant, this information allows for an understanding of the “face of the tumor,” which researchers can use to predict how the cell population developed and how it will continue to progress. This understanding is further improved upon by using the techniques to evaluate cell populations at different points in time, which provides insight into genetic changes of cells included in a malignant cell population over time. Additionally or alternatively, the information can be used to identify neoantigens associated with the cell population to identify or develop immunotherapies that specifically target the diseased tissue.

Consequently, the techniques developed by the inventors provide for more accurate identification and analysis of malignant cell populations in a biological sample than previously possible using conventional methods. This technology therefore provides an improved diagnostic tool, which can be used to improve the way in which treatments are identified for patients thereby improving clinical outcomes. The techniques described herein allow for the detection of malignant cell populations in early or post-treatment stages of a tumor, where conventional approaches, which identify malignant cell populations based on the size of the largest cell population alone, fail to do so. And even where such techniques are able to identify a cell population including malignant cells, the techniques developed by the inventors go further and identify cell populations including cells that might be otherwise excluded from the cell populations defined using conventional techniques.

The techniques described herein may be implemented as part of a software diagnostic tool, which may be used to present medical professionals with information identifying malignant cell populations and other cell populations in the biological sample. In turn, the software tool may use this information to generate a visualization of the cell populations and a visual indication of a cell population, within the cell populations, which includes malignant cells of a particular type (e.g., using color, shading, size, or any other suitable visual cue, as aspects of the technology described herein are not limited in this respect). Additionally, the visualization may include information about the sequencing data associated with each cell population.

Accordingly, some embodiments provide for computer-implemented techniques for identifying a malignant cell population. In some embodiments, the techniques include: (A) obtaining sequencing data (e.g., RNA-seq data, next generation sequencing (NGS) data) previously obtained from a biological sample (e.g., a biopsy, saliva, blood, etc.) from a subject (e.g., a subject having, suspected of having, or at risk of having cancer (for example, lymphoma) or an immune-related disease (for example, rheumatoid arthritis); (B) processing the sequencing data to identify: (1) a plurality of cell population estimates for a cell of a first type (e.g., B cells or T cells), the plurality of cell population estimates including a first cell population estimate and a second cell population estimate associated respectively with largest and second largest cell population estimates (e.g., determined based on a number or fraction of sequence reads associated with each cell population estimate) from among the identified plurality of cell population estimates; and (2) features associated with the plurality of cell population estimates, the features including: (a) a first feature indicative of a size of the first cell population estimate (e.g., a number or fraction of sequence reads associated with the largest cell population estimate); and (b) a second feature indicative of a ratio between sizes of the first cell population estimate and the second cell population estimate (e.g., ratio between the number or fraction of sequence reads associated with the largest estimate cell population and the number or fraction of sequence reads associated with the next largest cell population); and determining, using the features and a trained machine learning model (e.g., a naïve Bayes classifier, a support vector machine (SVM) classifier, a decision tree classifier, a random forest classifier, a neural network classifier, a non-linear regression classifier, a logistic regression classifier, an ensemble classifier (for example, an Adaboost classifier), etc.), whether the first cell population estimate includes malignant cells of the first type.

In some embodiments, processing the sequencing data to identify the plurality of cell population estimates includes: obtaining an initial estimate of cell populations (e.g., by grouping sequence reads associated with an identical region of a receptor chain); and generating the plurality of cell population estimates (e.g., by applying clustering techniques to the sequence reads) based on the initial estimate, wherein the initial estimate is different from the plurality of cell population estimates.

In some embodiments, the sequencing data includes a plurality of sequence reads and obtaining the initial estimate of cell populations further includes grouping sequence reads into groups based on similarity among sequence reads in the plurality of sequence reads (e.g., by grouping sequence reads associated with identical CDR3 regions).

In some embodiments, the initial estimate of cell populations includes multiple initial cell population estimates and obtaining the initial estimate of cell populations comprises obtaining, for each particular initial cell population estimate of at least some of the multiple initial cell population estimates: information indicative of a receptor chain (e.g., IgH, IgK, IgL, TRA, TRB, TRD, and TRG) associated with the particular initial cell population estimate; and sequence reads (e.g., associated with CDR3 and/or V(D)J regions) associated with the particular initial cell population estimate.

In some embodiments, at least some sequence reads of the sequence reads associated with the particular initial cell population correspond to identical complementarity determining regions (CDR3). For example, the sequence reads may align to the same CDR3 region. In some embodiments, generating the plurality of cell population estimates further includes clustering the sequence reads associated with at least some of the multiple initial cell population estimates. For example, the sequence reads may be clustered based on similarity between the CDR3 region and/or the V(D)J region. Clustering the sequence reads may include using any suitable clustering techniques, as aspects of the technology described herein are not limited in this respect.

Some embodiments further include determining a size of each cell population estimate of the plurality of cell population estimates based on a number of sequence reads associated with each initial cell population estimate (e.g., by comparing the number of sequence reads associated with an estimate cell population to the total number of sequence reads associated with all cell population estimates.)

In some embodiments, receptor chains may include an immunoglobulin heavy chain (IgH) and/or an immunoglobulin light chain. The light chain may be a kappa light chain (IgK) or a lambda light (IgL) chain.

In some embodiments, the plurality of cell population estimates comprises a first set of cell population estimates generated for IgH, a second set of cell population estimates generated for IgK, and a third set of cell population estimates generated for IgL.

In some embodiments, the first set includes the first cell population estimate and the second cell population estimate. In some embodiments, the second set includes a third cell population estimate and a fourth cell population estimate associated respectively with largest and second largest cell population estimates from among the second set. In some embodiments, the third set includes a fourth cell population estimate and a fifth cell population estimate associated respectively with largest and second largest cell population estimates from among the third set. In some embodiments, the first and second cell population estimates may be the largest and second largest cell population estimates defined based on similarity among sequence reads aligning to IgH receptor chains. The third and fourth cell population estimates may be the largest and second largest cell population estimates defined based on similarity among sequence reads aligning to IgK receptor chains. The fifth and sixth cell population estimates may be the largest and second largest cell population estimates defined based on similarity among sequence reads aligning to IgL receptor chains.

Some embodiments further include processing the sequencing data to identify features associated with the plurality of cell population estimates, the features including: a third feature indicative of a size of the third cell population estimate (e.g., fraction or number of sequence reads associated with the largest estimate cell population); and a fourth feature indicative of a ratio (e.g., ratio between the fraction or number of sequence reads associated with the largest estimate cell population and the fraction or number of sequence reads associated with the next largest cell population) between sizes of the third cell population estimate and the fourth cell population estimate; and determining, using the features and a trained machine learning model (e.g., a naïve Bayes classifier, a support vector machine (SVM) classifier, a decision tree classifier, a random forest classifier, a neural network classifier, a non-linear regression classifier, a logistic regression classifier, an ensemble classifier (for example, an Adaboost classifier), etc.), whether the third cell population estimate includes malignant cells of the first type.

Some embodiments further include processing the sequencing data to identify features associated with the plurality of cell population estimates, the features including: a fifth feature indicative of a size of the fifth cell population estimate (e.g., fraction or number of sequence reads associated with the largest estimate cell population); and a sixth feature indicative of a ratio (e.g., ratio between the fraction or number of sequence reads associated with the largest estimate cell population and the fraction or number of sequence reads associated with the next largest cell population) between sizes of the fifth cell population estimate and the sixth cell population estimate; and determining, using the features and a trained machine learning model (e.g., a naïve Bayes classifier, a support vector machine (SVM) classifier, a decision tree classifier, a random forest classifier, a neural network classifier, a non-linear regression classifier, a logistic regression classifier, an ensemble classifier (for example, an Adaboost classifier), etc.), whether the third cell population estimate includes malignant cells of the first type.

Some embodiments further include: obtaining coverages (e.g., value obtained through sequencing procedure) of the second and third sets of cell population estimates; and determining, based on the coverages and the third and fifth features (e.g., by comparing the coverages and features to specified criteria), whether to output a first result of determining whether the third cell population includes malignant cells of a first type, a second result of determining whether the fifth cell population includes malignant cells of the first type, or neither the first nor the second result.

In some embodiments, the sequencing data comprises RNA sequencing data.

In some embodiments, the sequencing data comprises raw DNA or RNA sequencing data, DNA exome sequencing data, DNA genome sequencing data, gene sequencing data, bias-corrected gene sequencing data, any sequencing data comprising data obtained from a sequencing platform, or any sequencing data derived from data obtained from a sequencing platform.

Some embodiments further comprise, prior to processing the sequencing data, filtering the sequencing data to exclude samples with a coverage (e.g., value obtained through the sequencing procedure) below a specified coverage threshold, wherein the specified coverage threshold is between 10 and 70.

In some embodiments, the trained machine learning model may be trained using RNA sequencing data obtained for biological samples from a plurality of subjects (e.g., healthy donors, patients diagnosed with a disease, etc.). The biological samples may comprise non-tumor samples and tumor samples that are diagnosed with cancer (e.g., lymphoma, B cell lymphoma, Hodgkin's lymphoma, non-Hodgkin's lymphoma, T cell lymphoma, etc.) In some embodiments, the trained machine learning model may be trained using sequencing data previously obtained from biological samples comprising B cells.

In some embodiments, the trained machine learning model was trained using sequencing data previously obtained from biological samples comprising cells with associated receptor chains that include IgH.

Some embodiments further comprise generating a graphical user interface (GUI) including a visualization indicating a result of processing the sequencing data, the visualization comprising a plurality of nodes including a first set of nodes, the first set of nodes representing a cell population estimate of the plurality of cell population estimates, wherein each node included in the first set of nodes represents a respective initial cell population estimate of the initial estimate of cell populations. Additionally or alternatively the visualization may indicate a result of identifying a malignant cell population of the plurality of cell population estimated. In some embodiments, the visualization includes a plurality of nodes and a plurality of edges, the plurality of nodes including a first set of nodes comprising a first node and a second node and the plurality of edges including a first edge connecting the first node and the second node.

In some embodiments, the first set of nodes includes a first node representing a first initial cell population estimate of the initial estimate of cell populations and a second node representing a second initial cell population estimate of the initial estimate of cell populations, wherein the first node is connected to the second node by an edge.

In some embodiments, a visual characteristic associated with at least some of the nodes in the first set of nodes is indicative of a characteristic of the first cell population estimate. In some embodiments, the visual characteristic associated with the at least some nodes in the first set of nodes comprises a respective size of each of the at least some nodes, a shading of each of the at least some nodes, and/or a color of each of the least some nodes. For example, sizes of nodes in the first set of nodes may indicate the size of the first cell population estimate relative to other cell population estimates.

In some embodiments, sequencing data comprises at least 1 million sequence reads, at least 5 million sequence reads, at least 10 million sequence reads, at least 20 million sequence reads, at least 50 million sequence reads, or at least 100 million sequence reads.

Cell Population Estimates

“Cell population estimates” are discussed with respect to some embodiments described herein. Sequencing data (e.g., RNA-seq data) may be obtained for a biological sample that includes regions of sequencing data associated with receptor chains (e.g., CRD3) of cells in the biological sample. Such regions are unique to a cell population (e.g., all cells in the cell population share identical sequencing data in these regions or sequencing data that is within a threshold distance, such as for example, an edit distance). As a result, the sequencing data may be used to reconstruct representative cell populations in the biological sample, resulting in a number of cell population estimates.

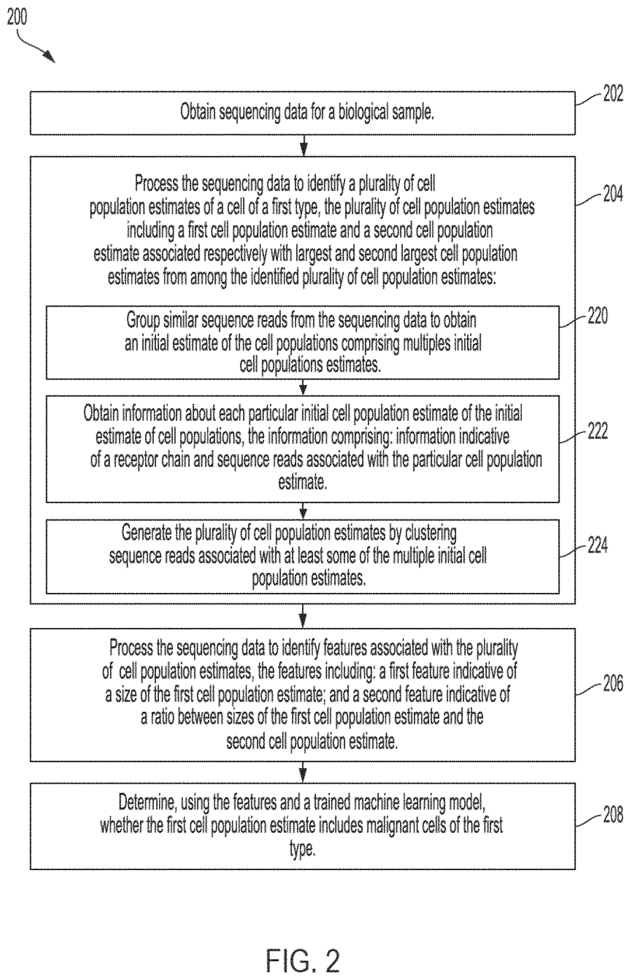

As described herein, generating the cell population estimates includes (a) obtaining an initial estimate of cell populations and (b) generating the cell population estimates based on the initial cell population estimates. In some embodiments, obtaining the initial estimate of cell populations includes grouping sequence reads associated with regions of the genome (e.g., the CDR3 and/or V(D)J regions) that are within a threshold distance of one another (for example, sequence reads with identical CDR3 and/or V(D)J regions). Sequencing reads associated with at least some of the initial cell population estimates are then clustered to generate the cell population estimates. For example, clustering techniques are described herein including at least with respect to FIG. 2.

Each resulting group of sequence reads may represent a respective cell population estimate. An indication of the size of a cell population estimate may be determined by comparing the number of sequence reads associated with the cell population estimate to the total number of sequence reads associated with all of the cell population estimates (e.g., determining the fraction of sequence reads associated with a cell population estimate).

As such, the cell population estimates described herein are estimate representations of the cell populations of the biological sample.

Receptor Chains

As discussed above, the sequence reads that are associated with receptor chains may be used to generate cell population estimates. In some cases, cells may include more than one receptor chain. For example, B cells each include an IgH chain and either an IgL chain or IgK chain. In some embodiments, sequence reads from each receptor chain may be used independently to generate cell population estimates. For example, in some embodiments, only the sequence reads from IgH may be used to generate cell population estimates for the identification of a malignant cell population. In other embodiments, the receptor chains may be analyzed together to identify a malignant cell population. As an example, an estimate cell population may be generated and subsequently analyzed using sequence reads from both a heavy chain and a light chain, such as IgH and IgL.

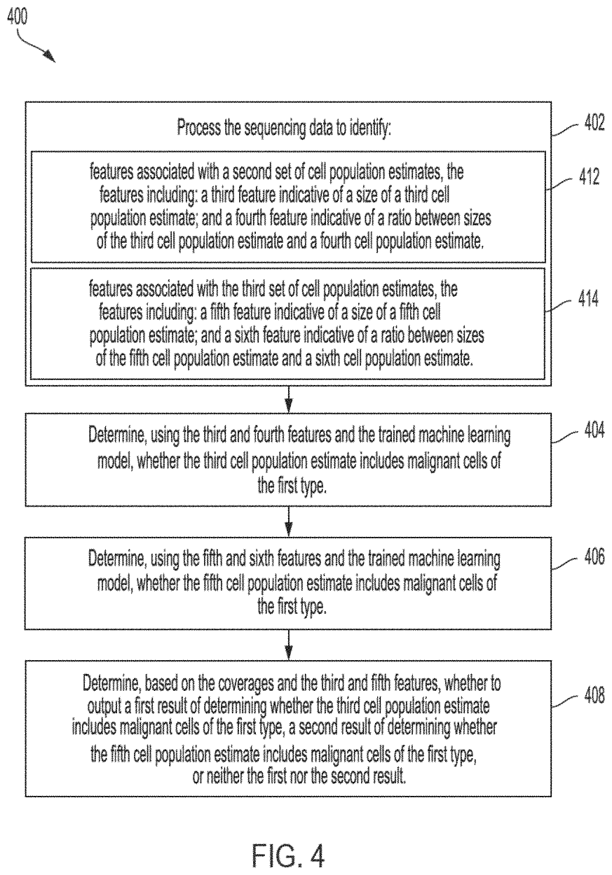

In some embodiments, a biological sample of B cells may include cells that have an IgL receptor chain and other cells that have an IgK receptor chain. As a result, cell population estimates that are generated using sequence reads for IgL will not account for cell populations that have IgK receptor chains, and vice versa. When generating estimate cell populations using either light chain (e.g., IgL and IgK), it is important to account for the cells that do not have that light chain (e.g., because they have the other light chain). Left unaccounted for, the resulting cell population estimates may be inaccurate. For example, the largest estimate cell population generating using one light chain (e.g., IgK) may not be the largest estimate cell population when accounting for cell populations having the other light chain (e.g., IgL). By extension, the incorrect estimate cell population may be identified as the malignant cell population. As such, as described herein, including at least with respect to FIGS. 4-7E, the inventors have developed systems and methods that account for the possible discrepancies.

Alternatively, one receptor chain may be expressed for all cells of a particular type, and therefore may be used to reliably generate cell population estimates and identify a malignant cell population. For example, the IgH chain is expressed for all B cells, therefore all cell populations may be accounted for in the cell population estimates generated using sequence reads that align to IgH receptor chains.

Following below are more detailed descriptions of various concepts related to, and embodiments of, the malignant cell population identification systems and methods developed by the inventors. It should be appreciated that various aspects described herein may be implemented in any of numerous ways. Examples of specific implementations are provided herein for illustrative purposes only. In addition, the various aspects described in the embodiments below may be used alone or in any combination and are not limited to the combinations explicitly described herein.

FIGS. 1A and 1B depicts a system 100 for identifying one or more malignant cell populations 110 based on sequencing data 106. As described herein, at least with respect to FIG. 14, the illustrated system may be implemented in a clinical or laboratory setting.

As shown in FIG. 1A, the system 100 involves sequencing a biological sample 102 using a sequencing platform 104, which may be used to produce sequencing data 106. The biological sample 102 may be obtained for a subject having, suspected of having, or at risk of having cancer or any immune-related disease. A subject may be at risk of having cancer, for example, if the subject has a genetic predisposition (e.g., a known genetic mutation or mutations) to cancer or may have been exposed to cancer-causing agents. Similarly, a subject may be at risk of having an immune-related disease if the subject has a genetic disposition to the immune-related disease or may have been exposed to environmental agents.

In some embodiments, the biological sample 102 may be obtained by performing a biopsy or obtaining a blood sample, a salivary sample, or any other suitable biological sample from the patient. The biological sample may have been previously obtained from a subject. Thus, any step applied to the sample (e.g., obtaining sequencing data from the biological sample) may be performed in vitro. The biological sample 102 may include diseased tissue (e.g., a tumor), and/or healthy tissue. In some embodiments, the biological sample 102 may be obtained from a physician, hospital, clinic, or other healthcare provider. In some embodiments, the origin or preparation methods of the biological sample may include any of the embodiments described herein including in the section called “Biological Samples”.

In some embodiments, the sequencing platform 104, which may produce sequencing data 106, may be a next generation sequencing platform (e.g., Illumina®, Roche®, IonTorrent®, etc.), or any high-throughput or massively parallel sequencing platform. In some embodiments, the sequencing platform 104 may include any suitable sequencing device and/or any sequencing system including one or more devices. In some embodiments, the sequencing methods may be automated, in some embodiments, there may be manual intervention. In some embodiments, the sequencing data 106 may be generated using other types of sequencing techniques (e.g., Sanger sequencing). In some embodiments, the sample preparation may be according to manufacturer's protocols. In some embodiments, the sample preparation may be custom made protocols, or other protocols which are for research, diagnostic, prognostic, and/or clinical purposes. In some embodiments, the protocols may be experimental.

Sequencing data 106 can include the sequencing data generated by a sequencing protocol (e.g., the series of nucleotides in a nucleic acid molecule identified by next-generation sequencing, Sanger sequencing, etc.) as well as information contained therein (e.g., information indicative of source, tissue type, etc.) which may also be considered information that can be inferred or determined from the sequencing data. Sequencing data 106 may be obtained using any suitable techniques, such as those described herein including in the section called “Sequencing Data” and “Alignment and Annotation.”

In some embodiments, the sequencing data 106 may be obtained in a text-based file (e.g., in a FASTA, FASTQ, BAM, or SAM format). In some embodiments, a file in which sequencing data is stored may contain quality scores of the sequencing data. In some embodiments, a file in which sequencing data is stored may contain sequence identifier information. In some embodiments, a file in which sequencing data is stored may contain a description. In some embodiments, a file in which sequencing data is stored may contain an aligned position. However, it should be appreciated that a file in which sequencing data is stored may contain any other suitable information, as aspects of the technology described herein are not limited to any particular type of information.

As one illustrative example, in some embodiments, the sequencing data may comprise bulk sequencing data. The bulk sequencing data may comprise at least 1 million reads, at least 5 million reads, at least 10 million reads, at least 20 million reads, at least 50 million reads, or at least 100 million reads. In some embodiments, the sequencing data comprises bulk RNA sequencing (RNA-seq) data, single cell RNA sequencing (scRNA-seq) data, or next generation sequencing (NGS) data. In some embodiments, the sequencing data comprises microarray data. In some embodiments, the sequencing data 106 includes any suitable sequencing data, such as the sequencing data described herein including in the section called “Sequencing Data.”

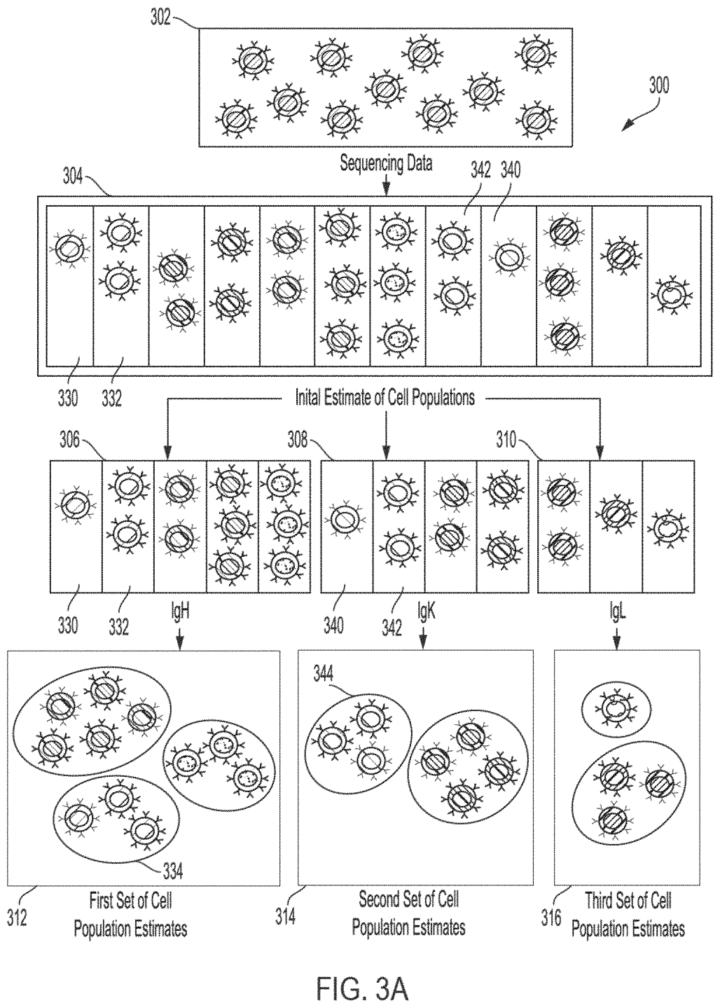

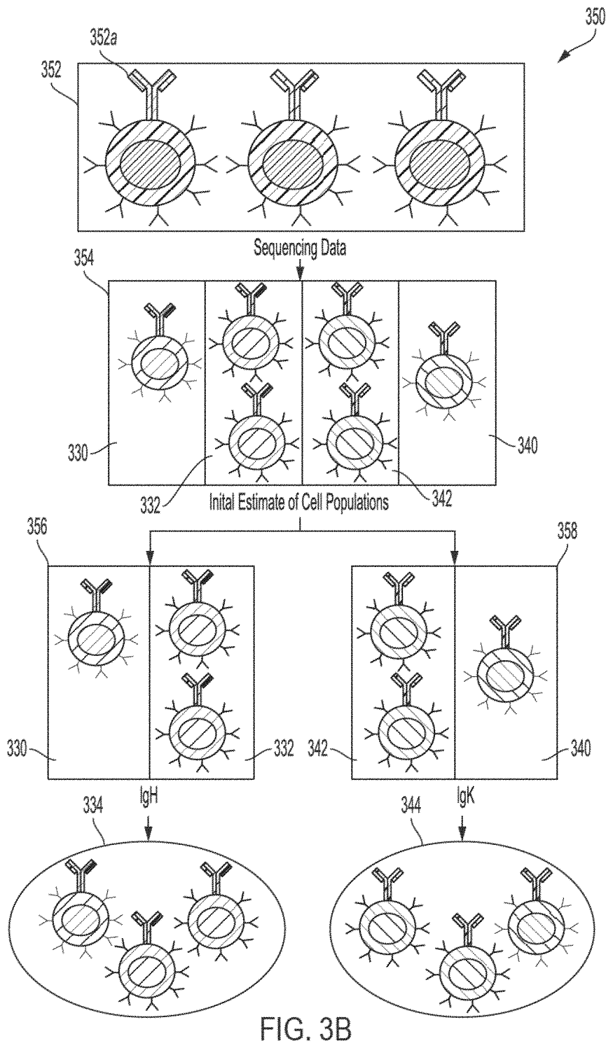

In some embodiments, the sequencing data 106 may be processed using computing device 108 in order to identify one or more malignant cell populations 110. For example, the sequencing data 106 may be processed by one or more software programs running on computing device 108 (e.g., as described herein with respect to FIG. 15). For example, the sequencing data 106 may be processed according to FIG. 1B, the cell population estimation techniques of FIGS. 2 and 3A-B, the machine-learning based approach of FIG. 2, and/or any other methods described herein for identifying malignant cell populations. In some embodiments, the computing device 108 may be operated by a user such as a doctor, clinician, researcher, patient, or other individual. For example, the user may provide the sequencing data 106 as input to the computing device 108 (e.g., by uploading a file), and/or may provide user input specifying processing or other methods to be performed using the sequencing data 106.

Regardless of how the sequencing data 106 is processed, the result 110 may be the identification of a malignant cell population from among multiple cell population estimates. As described herein, the malignant cell population represents a cell population that is cancerous or diseased (e.g., has an immune-related disease).

In some embodiments, a cell population estimate represents a population of cells in the biological sample that originate from the same cell. In some embodiments, cells originating from the cell include membrane receptor chains that bind to the same antigen or antigens. For example, cells in the same estimate cell population may include membrane receptor chains that include identical or similar (e.g., less than a threshold number of differences) CDR3 and/or V(D)J regions, which facilitate the binding of antigens. In some embodiments, the estimate cell populations each include cells of the same type. For example, the estimate cell populations may include B cells. Additionally or alternatively, the estimate cell populations may include T cells.

As an example, system 100 may be performed to identify one or more malignant cell populations in a patient suspected of having B cell lymphoma. The system 100 may include obtaining a blood sample, previously obtained by a physician. The blood sample may include healthy and/or diseased tissue (e.g., malignant cell populations and/or healthy cell populations). The system 100 may include processing the blood sample to extract RNA, which may be analyzed by Next Generation Sequencing (NGS) to produce RNA-seq data. In some embodiments, the RNA-seq data is processed, as described with respect to FIG. 1B, to identify the cell population estimates. The techniques described herein, including at least with respect to FIGS. 1B and 2 are used to identify the malignant cell populations. In some embodiments, these results are used to diagnose or treat the patient suspected of having B cell lymphoma. For example, a treatment may be developed or identified based on neoantigens specific to the identified malignant cell population.

FIG. 1B depicts an illustrative technique 120 for processing the sequencing data 106 on computing device 108 to identify one or more malignant cell populations 110, as discussed above with respect to FIG. 1A.

At act 112, the sequencing data 106 is processed to identify one or more cell population estimates 114. In some embodiments, processing the sequencing data 106 to identify the one or more cell populations includes (a) generating an initial estimate of cell populations and (b) generating the estimate cell populations based on the initial estimate of cell populations.

In some embodiments, processing the sequencing data 106 includes processing sequence reads. A sequence read is an inferred series of nucleotides corresponding to all or part of a fragment of DNA or a fragment of RNA. In some embodiments, the sequence reads included in sequencing data 106 correspond to the membrane receptor chains of cells in the biological sample. For example, the sequence reads may correspond to the CDR3 region and/or the V(D)J segment of membrane receptor chains. In some embodiments, the techniques include aligning sequence reads to a genome (e.g., a genome of an organism, such as a human) to determine whether the reads correspond to a particular region.

In some embodiments, processing the sequence reads to generate the initial estimate of cell populations may include grouping sequence reads that align to identical regions. For example, generating the initial estimate of cell populations may include grouping sequence reads that align to identical CDR3 regions. Accordingly, the initial estimate of cell populations may represent cells with membrane receptor chains that have not undergone the slight genetic mutations caused by environmental factors.

In some embodiments, after generating the initial estimate of cell populations and prior to generating the subsequent estimate cell populations, processing the sequencing reads 106 includes sorting the sequence reads based on the corresponding type of receptor chain. For example, for B cells, the techniques may include sorting sequence reads corresponding to IgH receptor chains, sequence reads corresponding to IgK receptor chains, and sequence reads corresponding to IgL receptor chains. Additionally or alternatively, for T cells, the techniques may include sorting sequence reads corresponding to TRA receptor chains, sequence reads corresponding to TRB receptor, sequence reads corresponding to TRD receptor chains, and sequence reads corresponding to TRG receptor chains.

In some embodiments, processing the sequence reads to generate the estimate cell populations includes processing sequence reads associated with one type of receptor chain for a cell type. For example, the techniques may include processing sequence reads corresponding to IgH receptor chains. Additionally or alternatively, processing the sequence reads to generate the initial and estimate cell populations includes processing sequence reads associated with multiple types of receptor chains for a cell type. However, the processing may be done independently. For example, the techniques may include processing sequence reads corresponding to IgH receptor chains to generate a first set of estimate cell populations for a sample and separately processing sequence reads associated with IgL receptor chains to generate a second set of estimate cell populations for the same sample.

In some embodiments, processing the sequence reads to generate the estimate cell populations includes clustering sequence reads associated with each of the cell populations of the initial estimate of cell populations. For example, the techniques may include applying the clustering techniques described herein including at least with respect to FIG. 2. Accordingly, in some embodiments, initial cell population estimates associated with sequence reads that have less than a threshold number of differences) are combined to generate an estimate cell population. Additional details for generating estimate cell populations are described herein including at least with respect to FIGS. 3A-B.

In some embodiments, the system 120 includes obtaining features based on the estimate cell populations 114. The features may be indicative of a size of the largest cell population estimate and a ratio between the sizes of the largest and second largest cell population estimates. In some embodiments, these features may be calculated using information derived from the sequencing data 106 during the processing act 112. For example, a size of an estimate cell population may be based on the number of sequence reads associated with the estimate cell population. Additionally or alternatively, the size of the estimate cell population may be based on the fraction of sequence reads associated with the estimate cell population relative the total number of sequence reads associated with all of the generated estimate cell populations 114.

In some embodiments, the features may be processed using a machine learning model 116 trained to determine whether one or more of the cell population estimates are malignant or non-malignant. For example, the machine learning model may include a naïve Bayes classifier, a support vector machine (SVM) classifier, a decision tree classifier, a random forest classifier, a neural network classifier, a non-linear regression classifier, a logistic regression classifier, an ensemble classifier (for example, an Adaboost classifier), or any suitable machine learning model, as aspects of the technology described herein are not limited to any particular machine learning technique.

FIG. 2 is a flowchart depicting a machine learning process 200 for identifying malignant cell populations based on sequencing data obtained from a biological sample, in accordance with some embodiments of the technology described herein. Process 200 may be performed by any suitable computing device(s). For example, process 200 may be performed by a laptop computer, a desktop computer, one or more servers, in a cloud computing environment, computing device 1500 as described herein with respect to FIG. 15, or in any other suitable way.

Process 200 begins at act 202, where sequencing data is obtained for a biological sample previously obtained from a subject having, suspected of having, or at risk of having cancer and/or an immune-related disease. Any suitable type of sequencing data may be obtained, such as the sequencing data described herein including at least with respect to FIG. 1A and the sequencing data described in the sections called “Sequencing Data” and “Alignment and Annotation,” or any suitable sequencing data, as aspects of the technology described herein are not limited to any particular type of sequencing data.

In some embodiments, obtaining sequencing data comprises obtaining sequencing data using a sequencing platform and/or from a data store storing such information. For example, the sequencing platform may include the sequencing platform described herein including at least with respect to FIG. 1A, or any other suitable sequencing platform, as aspects of technology are not limited in this respect. The data store may include any suitable data store, such as a flat file, a data store, a multi-file, or data storage of any suitable type, as aspects of the technology described herein are not limited to any particular type of data store. In some embodiments, the sequencing data obtained at act 202 includes sequence reads.

As described above, the sequence reads may correspond to regions of the membrane receptor chains of cells in the biological sample. For example, the sequence reads may correspond to the CDR3 region and/or the V(D)J segment of membrane receptor chains.

At act 204, the process 200 includes processing the sequencing data to identify a plurality of cell population estimates of a cell of a first type (e.g., B cells or T cells). The plurality of cell population estimates includes at least a first cell population estimate and a second cell population estimate associated respectively with largest and second largest cell population estimates. In some embodiments, the cell population estimates are generated by processing sequence reads corresponding to a particular receptor chain type (e.g., IgH, IgK, or IgL for B cells, TRA, TRB, TRD, or TRG for T cells). For example, cell population estimates for B cell populations in a biological sample may be generated by grouping sequence reads associated with the IgH receptor chain.

In some embodiments, act 204 includes sub-acts 220, 222, and 224. At sub-act 220, the process includes grouping similar sequence reads from the sequencing data to obtain an initial estimate of cell populations, which includes multiple initial cell population estimates. In some embodiments, the techniques may include grouping sequence reads corresponding to identical regions of a receptor chain. For example, sequence reads corresponding to identical CDR3 regions may be grouped to define an initial set of cell population estimates.

In some embodiments, in order to determine whether sequence reads correspond to particular regions of receptor chains, the techniques include aligning the sequence reads to a reference structure. For example, the techniques may include aligning the sequence reads to a genome or a portion of a genome, such as a human genome or the genome of an organism. In some embodiments, the alignment is performed using any suitable alignment techniques, such as, for example, those described in Bolotin et al. (MiXCR: software for comprehensive adaptive immunity profiling; Nature Methods, 2015, 12: 380-381). Bolotin et al. (MiXCR: software for comprehensive adaptive immunity profiling; Nature Methods, 2015, 12: 380-381) describes methods for processing sequencing data from raw sequence reads to generate quantitated clonotypes and is incorporated by reference herein in its entirety. In some embodiments, the alignment techniques are used to align at least 1 million sequence reads, at least 5 million sequence reads, at least 10 million sequence reads, at least 20 million sequence reads, at least 50 million sequence reads, or at least 100 million sequence reads

At sub-act 222, process 200 includes obtaining information about each particular initial cell population estimate of at least some of the multiple initial cell population estimates. In some embodiments, the information includes information indicative of the receptor chain (e.g., IgH, IgK, IgL, TRA, TRB, TRD, TRG). For example, such information may be obtained from sequence reads aligning to specific regions of the receptor chain (e.g., a constant region indicates the receptor chain type). In some embodiments, the information includes information indicative of the sequence reads associated with each particular initial cell population estimate. In some embodiments, the information indicates that the sequence reads align to certain regions of the receptor chains, such as the CDR3 regions and/or V(D)J regions. In some embodiments, the obtained information may be output from a software used to generate the initial cell population estimates, as described herein including with respect to FIGS. 3A-3B. In some embodiments, the information may be processed at act 224 to generate the plurality of cell population estimates.

Based on the information obtained at sub-act 222, and prior to sub-act 224, the techniques may include sorting the sequence reads, and by extension the initial cell populations estimates, based on their corresponding receptor chain type. For example, this may include distinguishing between initial cell population estimates associated with IgK, initial cell population estimates associated with IgL, and initial cell population estimates associated with IgH for a biological sample including B cells.

In some embodiments, sub-act 224 includes processing initial cell population estimates associated with one type of receptor chain. For example, this may include processing only the initial cell population estimates associated IgH (e.g., or by IgK, IgL, TRA, TRB, TRD, TRG, etc.) In particular, sub-act 224 includes generating the plurality of cell population estimates by clustering the sequence reads associated with at least some of the multiple initial cell population estimates (e.g., initial cell population estimates associated with one type of receptor chain). In some embodiments, prior to clustering, the step distance between initial cell population estimates (e.g., the number of substitutions needed to convert one sequence into another) is defined as the edit distance, which may be used to calculate a distance matrix. Next, in some embodiments, clustering is performed to generate the cell population estimates. For example, hierarchical clustering, with a specified height threshold, may be used to generate the cell population estimates. For example, the height threshold may be 1.0, 1.4, 1.6, 1.8, 1.9, 2.1, 2.2, 2.4, 2.6, 2.8, 3.0, or any suitable threshold, as aspects of the technology described herein are not limited in this respect. In some embodiments, only initial cell population estimates associated with identical V and J regions of the V(D)J region may be clustered together. In some embodiments, initial cell population estimates clustered into a same group using these techniques are considered to have a high degree of similarity.

At act 206, process 200 includes processing the sequencing data to identify features (e.g., a first feature and a second feature) associated with at least one set of the plurality of cell population estimates. In some embodiments, identifying the first feature, indicative of the size of the first cell population estimate, includes processing the sequencing data associated with the first cell population estimate. For example, as described herein, each cell population estimate may be associated with the sequence reads that were used to generate the cell population estimates. As such, the number of sequence reads associated with a cell population estimate may be indicative of the size of the cell population estimate. For example, the fraction of sequence reads associated with a cell population estimate, relative to the total number of sequence reads associated with all of the identified cell populations estimates, may be indicative of the size of that cell population estimate. In some embodiments, the second feature, indicative of a ratio between sizes of the first and second cell population estimates, is also determined based on the number of sequence reads associated with each of the first and second cell population estimates. For example, the second feature may include a ratio between the fraction of sequence reads associated with the second cell population estimate and the fraction of sequence reads associated with the first cell population estimate.

In some embodiments, at act 206, one or more additional features are optionally identified. For example, the techniques may include identifying the coverage of the estimate cell populations. In some embodiments, coverage may be indicative of an average number of sequence reads that align to certain regions of the receptor chains. In some embodiments, the coverage may be compared to a specified coverage threshold prior to act 208, to determine whether to exclude sequencing data (e.g., and, by extension certain cell population estimates) from further processing. For example, sequencing data with a coverage below a coverage threshold of 0, 20, 40, 60, 80, 100, 120, 140, 160, 180 or 200 may be excluded from act 208.

In some embodiments, the features identified for the cell population estimates may be embodied in at least one data structure having fields storing information indicative of the sizes of the largest and second largest cell population estimates. The data structure or data structures may be provided as input to software comprising code that is configured to access a machine learning model trained to determine whether the largest cell population estimate includes malignant cells of the first type, as described herein including with respect to act 208.

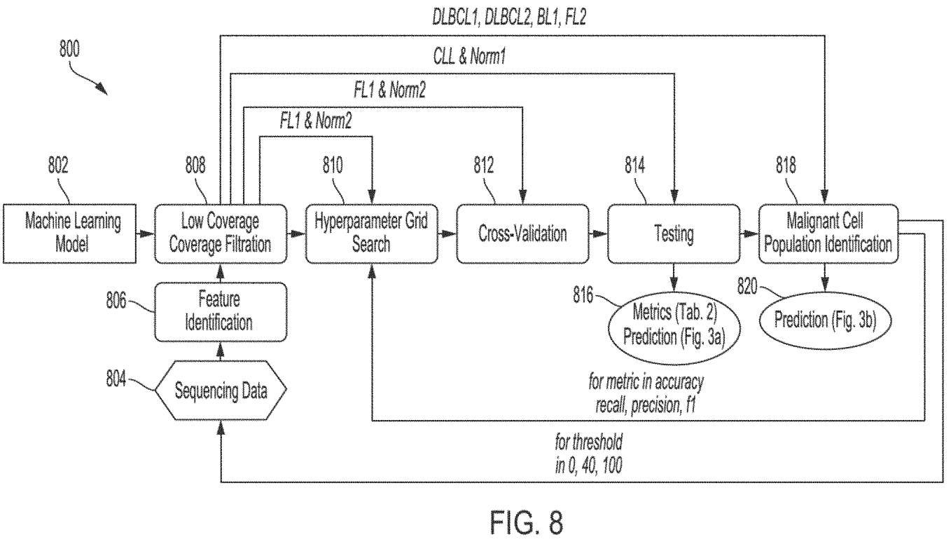

At act 208, the identified features and a trained machine learning model are used to determine whether the first cell population estimate includes malignant cells of the first type. The machine learning model may include, for example, a naïve Bayes classifier, a support vector machine (SVM) classifier, a decision tree classifier, a random forest classifier, a neural network classifier, a non-linear regression classifier, a logistic regression classifier, an ensemble classifier (for example, an Adaboost classifier), or any suitable machine learning model, as aspects of the technology described herein are not limited to any particular machine learning technique. In some embodiments, the machine learning model is trained according to the techniques described herein, including at least with respect to FIG. 8.

As described above, process 200 is used to generate a first set of cell population estimates at act 204 associated with a first receptor chain (e.g., identified prior to sub-act 224). The first set of cell population estimates are analyzed using the machine learning techniques to determine whether the largest cell population estimate of the first set of cell population estimates includes malignant cells of the first type (e.g., a first prediction for the biological sample).

However, it should be appreciated that one or more acts of process 200 may be repeated to generate a second prediction for the biological sample, based on a different receptor chain type. For example, process 200 may be used to generate a second set of cell population estimates at act 204 associated with a second receptor chain (e.g., identified prior to sub-act 224). The second set of cell population estimates may be analyzed using the machine learning techniques to determine whether the largest cell population estimate of the second set of cell population estimates includes malignant cells of the first type (e.g., a second prediction). This may be repeated for other receptor chains associated with cells in the biological sample to generate any suitable number of predictions, as aspects of the technology described herein are not limited in this respect. Techniques for analyzing second and third sets of cell population estimates are also described herein, including at least with respect to FIG. 4.

In some embodiments, each of the multiple predictions may be used, independently or in combination, to identify one or more malignant cell populations in the biological sample. In some embodiments, the predictions are used as quality control. For example, if the first prediction matches the second prediction, this may indicate that the techniques have correctly identified a malignant cell population.

Additionally or alternatively, some of the predictions may be more reliable than others. For example, as described above, some biological samples include B cells with IgK light chain receptors and B cells with IgL light chain receptors. Cell populations estimates generated based on sequencing data associated with one light chain may not accurately represent the largest cell population in the biological sample. However, the set of cell population estimates associated with the other light chain may accurately represent the largest cell population in the biological sample, and, by extension, the resulting prediction, may be more reliable. Accordingly, it may be desirable to select the more reliable prediction associated with the set of cell population estimates that most accurately represents the biological cell populations. FIGS. 4-7E describe example techniques for selecting between such predictions.

FIG. 3A is a diagram illustrating a technique 300 for processing the sequencing data to identify a plurality of cell population estimates including a first set of cell population estimates 312, a second set of cell population estimates 314, and a third set of cell population estimates 316, as described herein including with respect to act 204 of FIG. 2.

In some embodiments, sequencing data 302 is obtained from a biological sample previously obtained from a subject. The sequencing data 302 may include sequence reads that correspond (e.g., align) to certain regions of the receptor chains of cells in the biological sample. For example, the sequence reads may correspond to the CDR3 and/or V(D)J regions of the immunoglobulin heavy chains and light chains of B cells. In some embodiments, the sequence reads may correspond to regions of different receptor chains of the same cell. For example, some sequence reads may correspond to the CDR3 region of an IgH receptor chain of a cell, while other sequence reads may correspond to the CDR3 region of an IgK receptor chain of the same cell.

In some embodiments, the sequencing data 302 includes sequence reads. In some embodiments, the techniques include aligning the sequence reads to a reference structure (e.g., a genome, a portion of a genome, etc.) using any suitable techniques, as described herein including with respect to FIG. 2. For example, the alignment techniques may be used to determine the alignment of sequence reads to the CDR3 and/or V(D)J regions of a receptor chain.

In some embodiments, an initial estimate of cell populations 304 is generated based on the sequence reads included in the sequencing data 302. Sequence reads that are identical may be grouped into the same cell population estimate of the initial estimate of cell populations 304. For example, sequence reads aligning to identical CDR3 regions may be grouped to form the initial estimate of cell populations 304. In some embodiments, existing software may be used to generate the initial estimate of cell populations 304. For example, Bolotin et al. (MiXCR: software for comprehensive adaptive immunity profiling; Nature Methods, 2015, 12: 380-381) describes methods for processing sequencing data from raw sequence reads to generate quantitated clonotypes and is incorporated by reference herein in its entirety. In some embodiments, the quantitated clonotypes may be equivalent to the initial estimate of cell populations 304.

In some embodiments, information is obtained for each of the initial cell population estimates 304. For example, the information may include sequence reads used to define the initial cell population estimate (e.g., associated with the CDR3 region), an associated receptor chain, a number of sequence reads included in the initial cell population estimate, coverage, fraction of sequence reads associated with initial cell population estimate, and/or information about mutations in the sequencing reads associated with the initial cell population estimate. Information regarding the sequence reads may include specific nucleotide and amino acid sequences, V(D)J genes, and constant regions. In some embodiments, this information may be derived from the sequencing data or output by a software used for generating the initial estimate of cell populations 304.

In some embodiment, the initial cell population estimates 304 may then be sorted based on the obtained information, such as the associated receptor chain. For example, initial cell population estimates 330, 332 may each be associated with an IgH receptor chain. As a result, both may be sorted into the IgH receptor chain group 306. As another example, initial cell population estimates 340, 342 may each be associated with an IgK receptor chain. As a result, they both may be sorted into the IgK receptor chain group 308.

In some embodiments, the sequencing data associated with each receptor chain group 306, 308, 310 is processed to identify a plurality of cell population estimates, including a first set of cell population estimates 312 for cell populations associated with the IgH receptor chain, a second set of cell population estimates 314 for cell populations associated with the IgK receptor chain, and a third set of cell population estimates 316 for cell populations associated with the IgL receptor chain. The processing may include, for each receptor chain group 306, 308, 310, clustering sequencing reads associated with the initial cell population estimates according to the techniques described herein including at least with respect to FIG. 2. Initial cell population estimates with fewer than a threshold number of differences may be combined into one cell population estimate of the plurality of cell population estimates. For example, with the clustering techniques may group initial cell population estimate 330 and initial cell population estimate 332. As a result, the initial cell population estimates 330, 332 may be combined to form cell population estimate 334.

In some embodiments, the size and/or fraction may be calculated for each cell population estimate of each set of the plurality of cell population estimates 312, 314, 316. The size may be determined based on the number of sequence reads associated with each of the cell population estimates. The fraction may be determined based on the number of sequence reads associated with each cell population estimate compared to the total number of sequence reads associated with the set of cell population estimates. For example, if three sequence reads are associated with cell population estimate 334 and there are 11 total sequence reads associated with the first set of cell population estimates 312, then the fraction of cell population estimate 334, for the purpose of this example, would be 3/11.

Additionally, in some embodiments, as shown in FIG. 3A, technique 300 results in three sets of cell population estimates 312, 314, 316 that make up the plurality of cell population estimates. This is a result of having sequencing data 302 that includes sequence reads from each type of receptor chain (e.g., IgH, IgK, and IgL). In some embodiments, it may not be possible to identify which cell a sequence read is associated with. Therefore, multiple sets of cell population estimates may be generated (e.g., for each of the receptor chains) and analyzed separately. In other embodiments, it may be possible to identify the cell that a sequence read is associated with, allowing for the generation of cell population estimates that include sequence reads from both types of associated receptor chains (e.g., heavy and light chains).

In some embodiments, a set of cell population estimates for cell populations associated with a particular receptor may be embodied in at least one data structure having fields storing sequencing data associated with the set of cell population estimates and other information associated with the set of cell population estimates, such as, for example, the information described herein with respect to act 222 of FIG. 2.

FIG. 3B provides a non-limiting example of the technique 350 for identifying example cell population estimates. Sequencing data 352 may be obtained from a biological sample of three B cells from the same cell population. Each cell has an IgH receptor chain and an IgK receptor chain. For the purpose of this example, assume that a genetic mutation occurred in one of the B cells 352a, causing the CDR3 region of both its IgH and IgK receptor chains to vary slightly from the CDR3 regions of the receptor chains of the other two cells.

Sequence reads associated with the CDR3 regions may be used to generate the initial estimate of cell populations 354. In this example, identical CDR3 regions are used to define each initial cell population estimate 330, 332, 340, 342. As a result, sequence reads aligning to the CDR3 regions of the IgH receptor chains of the two cells that did not undergo the genetic mutation are grouped into initial cell population estimate 332, while the sequence reads aligning to CDR3 region of the IgH receptor chain of the cell that did undergo the genetic mutation are grouped into a separate initial cell population estimate 330. Similarly, sequence reads associated with the CDR3 regions of the IgK receptor chains of the two cells that did not undergo genetic mutation are grouped into initial cell population estimate 342, while the sequence reads including the CDR3 region of the IgK receptor chain of the cell that did undergo genetic mutation are grouped into a separate initial cell population estimate 340.

The initial cell population estimates 354 may then be further sorted based on the associated receptor chains. As such, initial cell population estimates 330, 334, that were generated based on sequence reads associated with the IgH receptor chain, are sorted into the IgH receptor chain group 356. The initial estimate cell population estimates 340, 342, that were generated based on sequence reads associated with the IgK receptor chain, are sorted into the IgK receptor chain group.

Finally, the sequence reads associated with each of the initial cell population estimates 356 generated for the IgH receptor chain may be clustered to determine cell population estimate 334, while each of the initial cell population estimates 358 generated for the IgK receptor chain may be clustered to determine cell population estimate 344. Since all three cells were derived from the same biological cell population, they all share very similar, though not identical, sequencing data. As shown, clustering may be used to reveal the high measure of similarity between these regions, and to ultimately group all of the sequence reads into a same cell population estimate 334, 344 for each of the associated receptor chains.

As shown through the example of FIG. 3B, technique 350 for generating cell population estimates may account for the variation among sequence reads caused by different mutational events, unlike conventional methods. As a result, the cell population estimates, generated using technique 350, are more biologically accurate. Not only does this improve the accuracy of the methods and systems described herein that rely on the output of technique 350, but it also improves existing diagnostic and treatment technologies that rely on the accurate reconstruction of cell populations.

As described herein, including at least with respect to FIGS. 2 and 3A-3B, the techniques developed by the inventors are used, in some embodiments, to identify multiple sets of cell population estimates. For example, the techniques may be used to generate a first set of cell population estimates for cell populations associated with IgH, a second set of cell population estimates for cell populations associated with IgK, and/or a third set of cell population estimates for cell populations associated with IgL.

Accordingly, the techniques developed by the inventors, in some embodiments, are used to output a prediction for each respective set of cell population estimates. For example, the techniques may be used to output an “IgH prediction,” which indicates whether the largest cell population estimate in the first set of cell population estimates includes malignant cells of the first type, an “IgK prediction,” which indicates whether the largest cell population estimate in the second set of cell population estimates includes malignant cells of the first type, and/or an

“IgL prediction,” which indicates whether the largest cell population estimate in the third set of cell population estimates includes malignant cells of the first type. Techniques for determining whether a largest cell population estimate of a set of cell population estimates includes malignant cells of a particular type are described herein including at least with respect to FIGS. 2 and 4.

It should be appreciated that, in some embodiments, some biological samples include B cell populations associated with only one type of light chain (e.g., either IgK or IgL), or that a vast majority of the B cell populations are associated with one type of light chain. Accordingly, the set of cell population estimates for cell populations associated with that light chain receptor are representative of the biological cell populations. For example, the set of cell population estimates accurately represents the number and relative of sizes of the biological cell populations.

Alternatively, some biological samples include some B cell populations associated with one type of light chain receptor (e.g., IgK) and some B cell populations associated with the other type of light chain receptor (e.g., IgL). Accordingly, the set of cell population estimates associated with cell populations associated with IgK may represent a portion of the biological cell populations (e.g., the second set of cell population estimates 314 as shown in FIG. 3A), while the set of cell population estimates associated with cell populations associated with IgL may represent the remaining proportion of cell population estimates (e.g., the third set of cell population estimates 316 as shown in FIG. 3A).

As a result, only one set of cell population estimates represents the true largest cell population in the biological sample. Therefore, while the techniques described herein, including at least with respect to FIGS. 2 and 3A-3B, may be used to output an IgK prediction and an IgL prediction, only one prediction may indicate whether the true largest cell population in the biological sample includes malignant cells of a particular type. Accordingly, it is desirable to identify the prediction that accurately captures this information. Additionally or alternatively, the sets of cell population estimates for cell populations associated with either IgK and/or IgL may be associated with sequencing data that has low coverage (e.g., below a threshold). Using sequencing data with low coverage may lead to inaccurate and unreliable predictions. Accordingly, the techniques described herein consider the coverage associated with each set of cell population estimates in identifying the prediction (e.g., the IgK or IgL prediction) that is to be output.

FIG. 4 is a flowchart depicting a process 400 for selecting a prediction associated with an immunoglobulin light chain, in accordance with some embodiments of the technology described herein.

At act 402, process 400 includes processing the sequencing data to identify features 412, associated with a second set of cell population estimates and features 414 associated with a third set of cell population estimates. In some embodiments, the second set of cell population estimates was generated for cell populations associated with a first light chain receptor (e.g., IgK). In some embodiments, the third set of cell population estimates was generated for cell populations associated with a third light chain receptor (e.g., IgL). For example, the second and third sets of cell population estimates may have been generated using the techniques described herein including at least with respect to FIGS. 2 and 3A-3B.

In some embodiments, the features 412 associated with the second set of cell population estimates include a feature indicative of a size of a third cell population estimate and a feature indicative of a ratio between sizes of the third cell population estimate and a fourth cell population estimate. For example, the third cell population estimate may be the largest cell population estimate and the fourth cell population estimate may be the second largest cell population estimate of the first set of cell population estimates.

In some embodiments, the features 414 associated with the third set of cell population estimates include a feature indicative of a size of a fifth cell population estimate and a feature indicative of a ratio between sizes of the fifth cell population estimate and a sixth cell population estimate. For example, the fifth cell population estimate may be the largest cell population estimate and the sixth cell population estimate may be the second largest cell population estimate of the second set of cell population estimates.

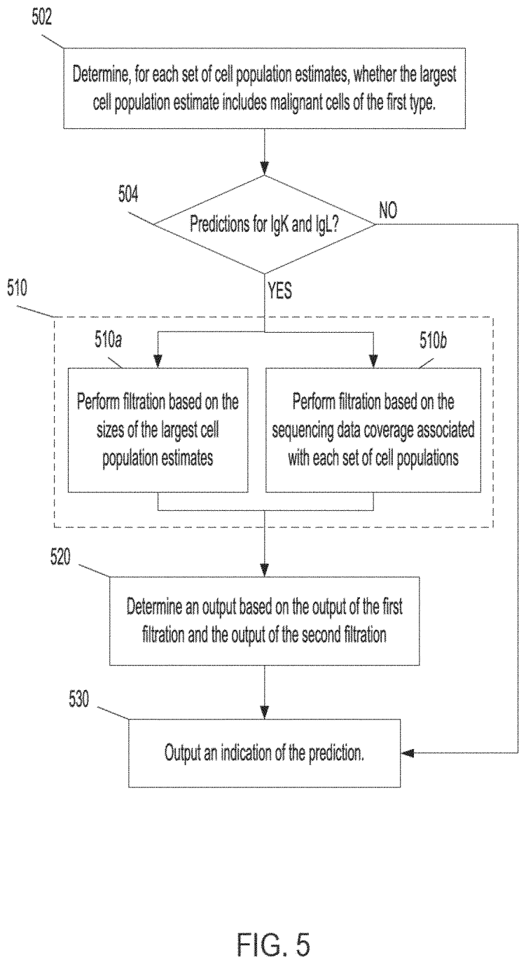

At act 404, process 400 includes processing the features associated with the second set of cell population estimates using a trained machine learning model to determine whether the third cell population estimate includes malignant cells of the first type. In some embodiments, act 404 includes the techniques described herein including with respect to act 208 of FIG. 2. In some embodiments, the output includes a prediction for the second set of cell population estimates. For example, the output may include an “IgK prediction” as referred to herein in connection with FIGS. 5-7E.

At act 406, process 400 includes processing the features associated with the third set of cell population estimates using the trained machine learning model to determine whether the fifth cell population estimate includes malignant cells of the first type. In some embodiments, act 406 includes the techniques described herein including with respect to act 208 of FIG. 2. In some embodiments, the output includes a prediction for the third set of cell population estimates. For example, the output may include an “IgL prediction” as referred to herein in connection with FIGS. 5-7E.

In some embodiments, acts 404 and 406 may be conducted in parallel or in the reverse order (e.g., act 406 may be conducted before act 404).

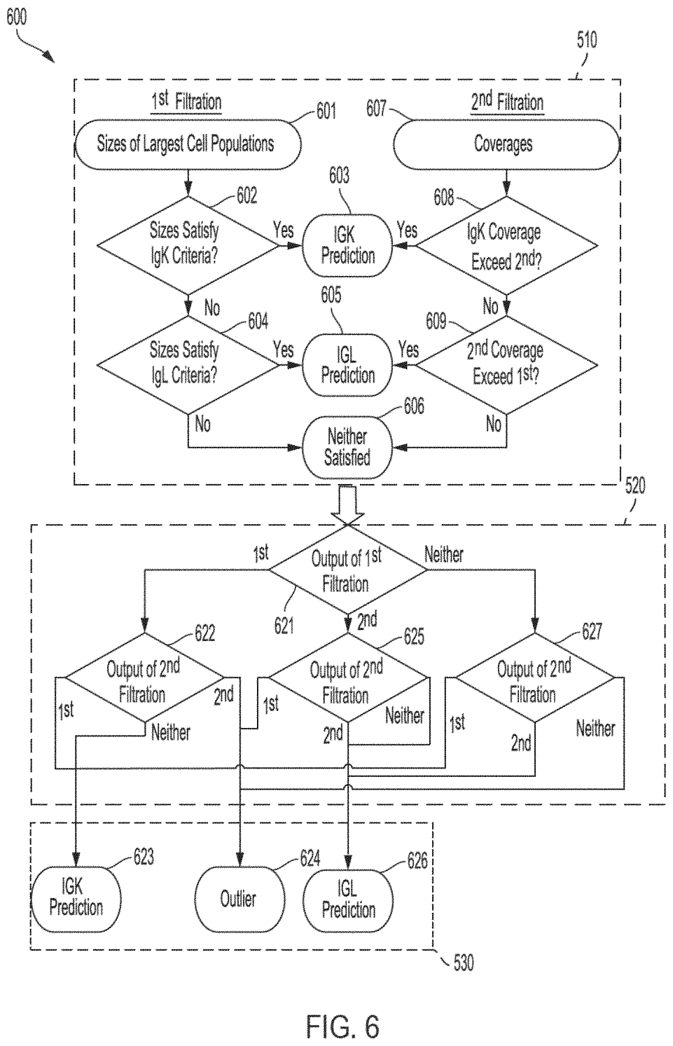

At act 408, process 400 includes determining, based on coverages and the features indicative of the sizes of the largest cell population estimates in each set of cell population estimates, whether to output a result of act 404 (e.g., the IgK prediction), a result of act 406 (e.g., the IgL prediction), or neither result. In some embodiment, the coverages may be obtained in parallel with act 402. In some embodiments, the coverages may be obtained as described herein including with respect to act 206 of FIG. 2. Example implementations of act 408 is described herein including at least with respect to FIGS. 5-6.

FIG. 5 is a flowchart illustrating an example of process 500 for selecting a prediction associated with an immunoglobulin light chain, according to some embodiments of the technology described herein.

Act 502 includes, in some embodiments, determining, for each set of cell population estimates, whether the largest cell population estimate includes malignant cells of the first type 502. In some embodiments, this includes generating a prediction for one or more sets of cell population estimates. For example, this may include determining whether the largest cell population estimate of a set of cell populations estimates for cell populations associated with IgK include malignant cells of a first type (e.g., an IgK prediction). Additionally or alternatively, this may include determining whether the largest cell population estimate of a set of cell population estimates for cell populations associated with IgL include malignant cells of the first type (e.g., an IgL prediction). Additionally or alternatively, this may include determining whether the largest cell population estimate of a set of cell population estimates for cell populations associated with IgH include malignant cells of the first type (e.g., an IgH prediction). In some embodiments, the techniques at act 502 may include the techniques described herein including with respect to act 208 of FIG. 2 and acts 404 and 406 of FIG. 4.

At act 504, the techniques include, in some embodiments, determining whether an IgK prediction and an IgL prediction was output at 504. If only one light chain prediction was output (e.g., either an IgK prediction or an IgL prediction), then process 500 proceeds to act 530, which includes outputting an indication of that prediction (e.g., either IgL or IgK).

If, at act 504, it is determined that both an IgK prediction and an IgL prediction were output, then process 500 proceed to act 510. In some embodiments, act 510 includes one or more filtration techniques. The filtration techniques may be used to process the set of cell population estimates for cell populations associated with IgK and the set of cell population estimates for cell populations associated with IgL. For example, act 510 includes sub-acts 510a, which includes performing filtration based on the sizes of the largest cell population estimates in each set of cell population estimates, and sub-act 510b, which includes performing filtration based on the sequencing data coverage associated with each set of cell populations. Embodiments of the filtration techniques at act 510 are described herein including at least with respect to FIG. 6.

At act 520, process 500 includes determining an output based on outputs of the filtration techniques 510 (e.g., the first filtration at act 510a and the second filtration at act 510b). In some embodiments, determining an output includes determining whether to output the IgK prediction or the IgL prediction, which were determined at act 502. Additionally or alternatively, the techniques may include determining to output neither the IgK nor the IgL prediction. Techniques for determining the output are described herein, including at least with respect to FIG. 6.

At act 530, process 500 includes outputting an indication of the prediction. In some embodiments, outputting the indication of the prediction may include using the prediction for further analysis (e.g., for identifying a treatment, monitoring tumor progression, etc.). In some embodiments, outputting an indication of the prediction includes generating a graphical user interface (GUI) including a visualization of the prediction. However, it should be appreciated that outputting the indication of the prediction 530 may include using any suitable techniques, as aspects of the technology described herein are not limited to any particular type of output or application.

FIG. 6 is an example flowchart of process 600 for selecting a prediction associated with an immunoglobulin light chain, in accordance with some embodiments of the technology described herein.

In some embodiments, process 600 includes a first stage 510, for performing filtration techniques, a second stage 520, for determining an output, and a third stage 530, for outputting an indication of the prediction. In some embodiments, the first stage 510 includes a first filtration and a second filtration. The results of each filtration are used to inform stage 620 for determining the output.

In some embodiments, the first filtration processes the size of the largest cell population estimate 601 in each set of cell population estimates. For example, this includes processing the size of the largest cell population estimate in the set of cell population estimates associated with IgK (referred to herein as the “size associated with IgK”) and the size of the largest cell population estimate in the set of cell population estimates associated with IgL (referred to herein as the “size associated with IgL”). In some embodiments, the first filtration includes comparing the sizes 601 to a set of thresholds at one or both of acts 602 and 604 to determine an output of the first filtration (e.g., the IgK prediction 603 or the IgL prediction 605).

At act 602, the size associated with IgK and the size associated with IgL are compared to a set of thresholds to determine if first criteria are satisfied. In some embodiments, the size associated with IgK is compared to a minimum size threshold (e.g., 0.1, 0.2, 0.3, or any suitable threshold) and the size associated with IgL is compared to a maximum size threshold (e.g., 0.4, 0.5, 0.6, or any suitable threshold). If the size associated with IgK exceeds the minimum size threshold and the size associated with IgL is less than the maximum size threshold, then the IgK prediction 603 is selected for the first filtration. If the criteria are not met, then the process 600 proceeds to act 604.

At act 604, the size associated with IgK and the size associated with IgL are compared to a different set of thresholds than at act 602 to determine if second criteria are satisfied. In some embodiments, the size associated with IgK is compared to a maximum size threshold (e.g., 0.4, 0.5, 0.6, or any suitable threshold) and the size associated with IgL is compared to a minimum size threshold (e.g., 0.1, 0.2, 0.3, or any suitable threshold). If the size associated with IgK is less than the maximum size threshold and the size associated with IgL exceeds the minimum size threshold, then the IgL prediction 605 is selected for the first filtration. If the criteria are not met, then the output of the first filtration indicates that neither criterion 606 was satisfied. In some embodiments, the second filtration processes the coverages 607 associated with each set of cell population estimates. For example, this includes processing the coverage associated with the set of cell population estimates associated with IgK (referred to herein as the “IgK coverage”) and the coverage associated with the set of cell population estimates associated with IgL (referred to herein as the “IgL coverage”). In some embodiments, the first filtration includes comparing the coverages 607 to a set of thresholds at one or both of acts 608 and 609 to determine an output of the second filtration (e.g., the IgK prediction 603 or the IgL prediction 605).

At act 608, the IgK coverage is compared to the IgL coverage to determine whether the IgK coverage exceeds the IgL coverage by a specified coverage factor. For example, the coverage factor may be 25, 50, 100, 125, 150, 175, or 200. If the IgK coverage exceeds the IgL coverage by the coverage factor, then the IgK prediction 603 may be selected for the second filtration. If not, then the second filtration proceeds to act 609

At act 609, the IgL coverage is compared to the IgK coverage to determine whether the IgL coverage exceeds the IgK coverage by a specified coverage factor. For example, the coverage factor may be 25, 50, 100, 125, 150, 175, or 200. If the IgL coverage exceeds the IgK coverage by the coverage factor, then the IgL prediction 605 is selected for the second filtration. If not, then the output of the second filtration indicates that neither criterion 606 was satisfied 606.

In some embodiments, the results of first stage 510 are used to inform stage 520, for determining an output. The output of stage 520 includes either an IgK prediction 623, an IgL prediction 226, or an indication that the sample as an outlier 624. The IgK prediction 623 indicates that the prediction for the set of cell population estimates associated with IgK should be output. The IgL prediction 626 indicates that the prediction for the set of cell populations associated with IgL should be output. An output indicating that the sample is an outlier indicates that neither the prediction for the set of cell population estimates associated with IgK, nor the set of cell populations associated with IgL should be used for further analysis.

At stage 520 the techniques include comparing the outputs of the first and second filtrations at stage 510 to determine whether to output the IgK prediction 623, IgL prediction 626, or neither. If neither result is output, the sample may be considered an outlier 624.

In some embodiments, if the output of the first filtration 621 is the IgK prediction 603 and the output of the second filtration 622 is the IgK prediction 603, then the IgK prediction 623 is output at stage 520. Similarly, if the output of the first filtration 621 is the IgL prediction 605 and the output of the second filtration 625 is the IgL prediction 605, then the IgL prediction 626 is output at stage 520.

In some embodiments, if the output of the first filtration 621 is the IgK prediction 603 and the output of the second filtration 622 is the IgL prediction 605, then neither the IgK nor the IgL prediction may be output at stage 520. This may apply vice versa if the output of the first filtration 621 is the IgL prediction 605 and the output of the second filtration 625 is the IgK prediction 603. Accordingly, the output of the second stage 520 indicates that the sample is an outlier 624.

In some embodiments, if the output of the first filtration 621 is the IgK prediction 603 and the output of the second filtration 622 is neither result 606, then the IgK prediction 623 is output at stage 520. Similarly, if the output of the first filtration 621 is the IgL prediction 605 and the output of the second filtration 625 is neither result 606, then the IgL prediction 626 is output at stage 520.

In some embodiments, if the output of the first filtration 621 is neither result 606 and the output of the second filtration 627 is the IgK prediction 603, then the IgK prediction 623 is output at stage 520. Similarly, if the output of the first filtration 621 is neither result 606 and the output of the second filtration 627 is the IgL prediction 605, then the IgL prediction 626 is output at stage 520.

In some embodiments, if the output of the first filtration 621 is neither result 606 and the output of the second filtration 627 is neither result 606, then neither the IgK prediction, nor the IgL prediction may be output at stage 520, and the sample is identified to be an outlier 624.

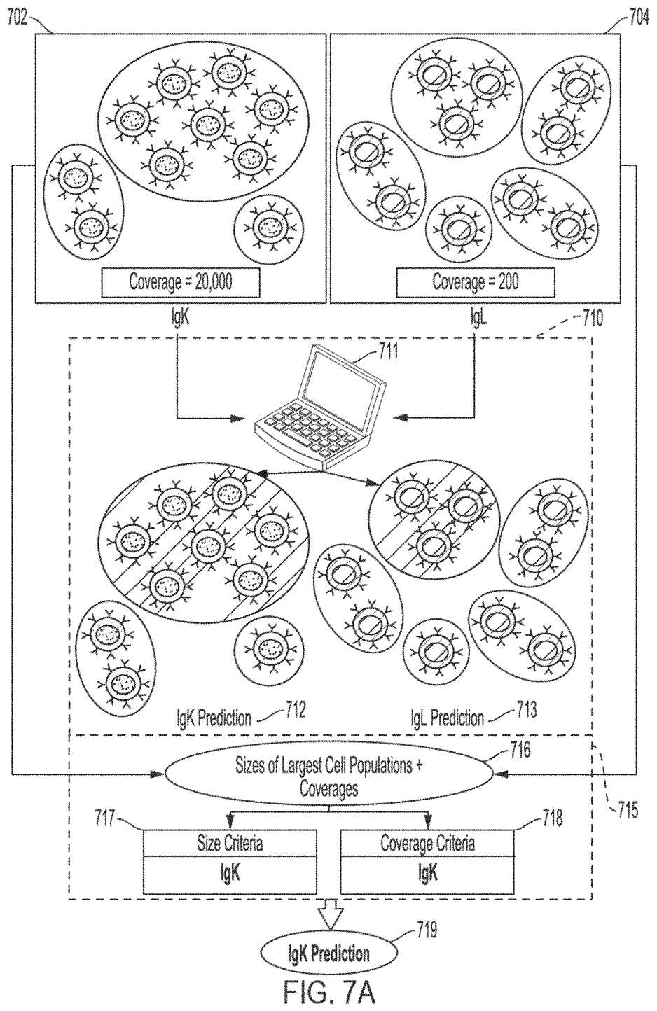

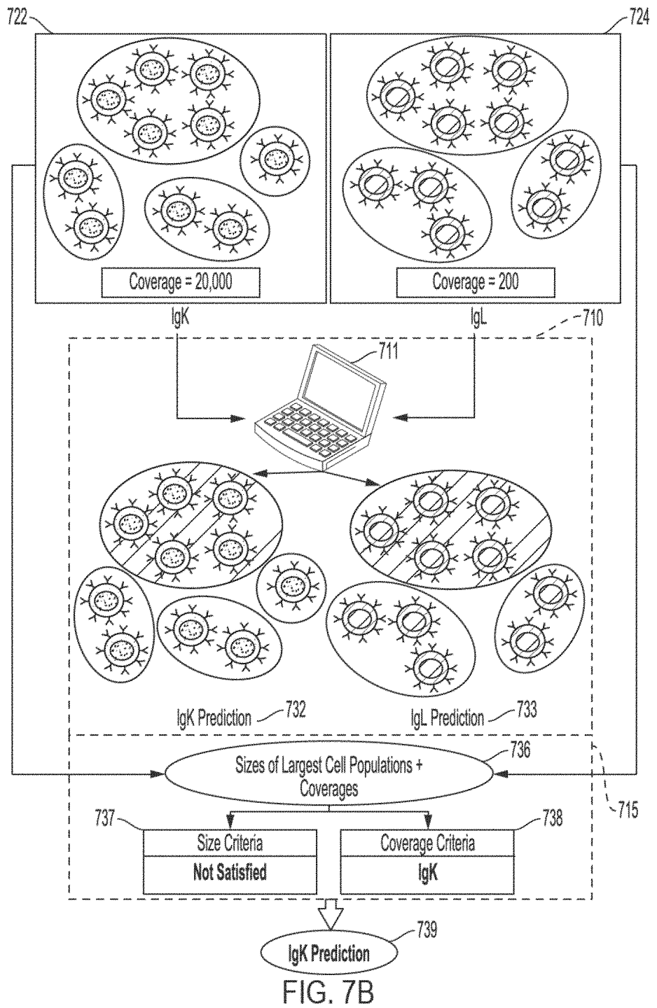

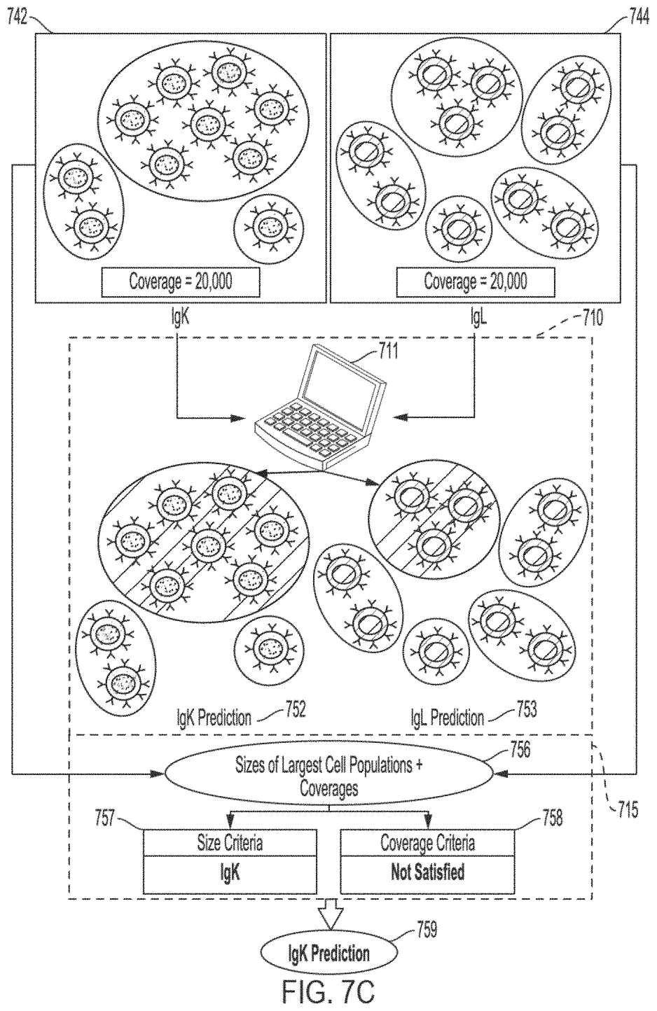

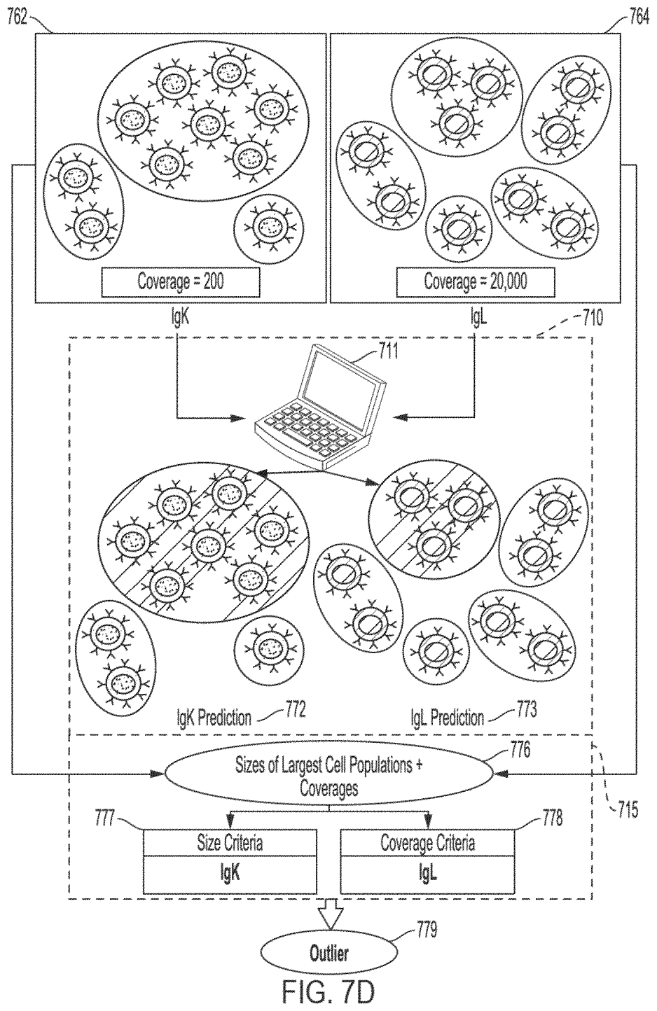

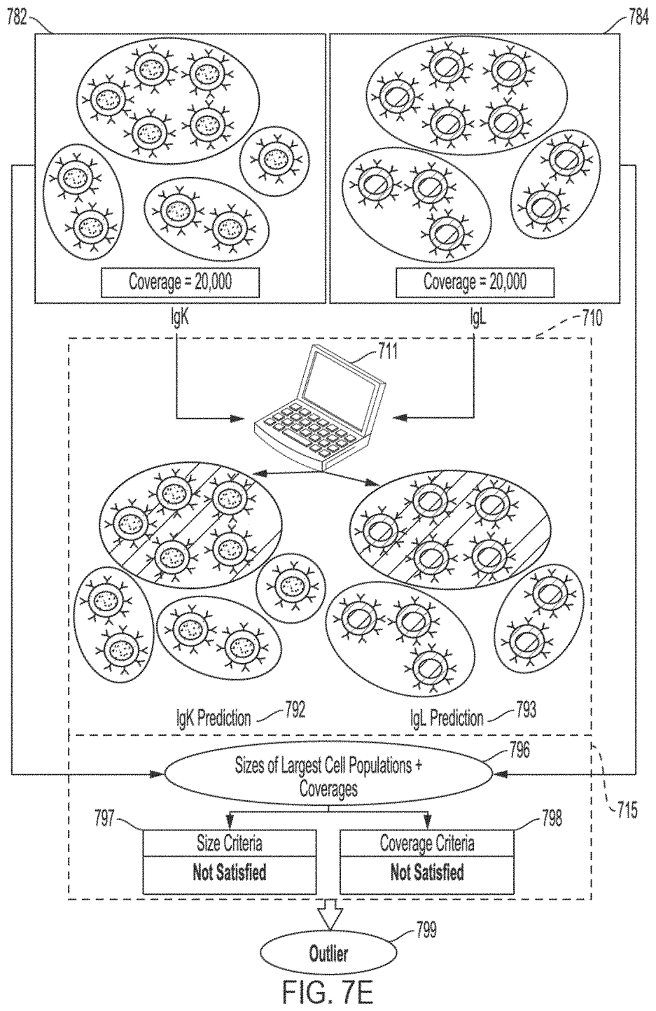

FIGS. 7A-E are illustrative examples of the processes described herein including at least with respect to FIGS. 4-6 for selecting a prediction associated with an immunoglobulin light chain for further analysis, in accordance with some embodiments of the technology described herein.

For the purpose of the following non-limiting examples, let the fraction of sequence reads associated with the largest cell population estimate of each set of cell population estimates be the feature indicative of size. Further, let the minimum size thresholds be 0.2, the maximum size thresholds be 0.5, and the specified coverage factor be 50.

The example in FIG. 7A includes a set of IgK cell population estimates 702 and a set of IgL cell population estimates 704. For the purpose of this example, let the fraction of sequence reads associated with the largest cell population estimate of the set of IgK cell population estimates 702 be 0.7 and the fraction of sequence reads associated with the largest cell population estimate of the set of IgL cell population estimates 704 be 0.3. Further, the coverages, as shown in FIG. 7A, are 20,000 and 200 for the set of IgK cell population estimates 702 and the set of IgL cell population estimates 704, respectively.

At stage 710, features for each set of cell population estimates 702, 704 are processed using a trained machine learning model 711 to determine whether the largest cell population estimate includes malignant cells of a first type. IgK prediction 712 corresponds to a result of determining whether the largest cell population estimate of the IgK cell population estimates 702 includes malignant cells of the first type. IgL prediction 713 corresponds to a result of determining whether the largest cell population estimate of the IgL cell population estimates 704 includes malignant cells of the first type.

At stage 715, the sizes of the largest cell population estimates and the coverages for each set of cell population estimates 702, 704 are used as input 716 to the two filtration steps, described herein including at least with respect to FIGS. 4-6.

For the first filtration step, the sizes of the largest cell population estimates are compared to the maximum and minimum size thresholds. In this example, 0.7, the size of the largest cell population estimate of the set of IgK cell population estimates 702, exceeds the minimum size threshold of 0.2. Additionally, 0.3, the size of the largest cell population estimate of the set of IgL cell population estimates 704, is less than the maximum size threshold of 0.5. Since this satisfies the first criteria (e.g., act 602 of FIG. 6), the IgK prediction 712 is selected as output 717 for the first filtration.

For the second filtration step, the coverages for each set of cell population estimates 702, 704 are compared to one another and to a coverage factor. In this example, the coverage of the set of IgK cell population estimates 702 exceeds the coverage of the set of IgL cell population estimates 704 by a factor of 100. Since this is greater than the example coverage factor of 50, this satisfies the first criteria (e.g., act 608 of FIG. 6), and the IgK prediction 712 is selected as output 718 for the second filtration.

Based on the outputs 717, 718 of the two filtration steps, a final output 719 is selected. Since, the IgK prediction was selected as the outputs 717, 718 for both filtration steps, the IgK prediction is selected as the final output 719. The example in FIG. 7B includes a set of IgK cell population estimates 722 and a set of

IgL cell population estimates 724. For the purpose of this example, let the fraction of sequence reads associated with the largest cell population estimate of the set of IgK cell population estimates 722 be 0.5 and the fraction of sequence reads associated with the largest cell population estimate of the set off IgL cell population estimates 724 be 0.5. Further, the coverages, as shown in FIG. 7B, are 20,000 and 200 for the set of IgK cell population estimates 722 and the set of

IgL cell population estimates 724, respectively.

At stage 710, features for each set of cell population estimates 722, 724 are processed using the trained machine learning model 711 to determine whether the largest cell population estimate includes malignant cells of a first type. IgK prediction 732 corresponds to determining that the largest cell population estimate of the set of IgK cell population estimates 722 includes malignant cells of the first type. IgL prediction 733 corresponds to determining that the largest cell population estimate of the set of IgL cell population estimates 724 includes malignant cells of the first type.

At stage 715, the sizes of the largest cell population estimates and the coverages for each set of cell population estimates 722, 724 are used as input 736 to the two filtration steps, described herein including at least with respect to FIGS. 4-6.

For the first filtration step, the sizes of the largest cell population estimates may be compared to the maximum and minimum size thresholds. In this example, 0.5, the size of the largest cell population estimate of the set of IgK cell population estimates 722, exceeds the minimum size threshold of 0.2. However, 0.5, the size of the largest cell population estimate of the set of IgL cell population estimates 724, exceeds the maximum size threshold of 0.5. Similarly, the size of the largest cell population estimate of the set of IgL cell population estimates 724 exceeds the minimum size threshold of 0.2, but the size of the largest cell population estimate of the set of IgK cell population estimates 722 exceeds the maximum size threshold of 0.5. Since this satisfies neither the first criteria (e.g., act 602 of FIG. 6) nor the second criteria (e.g., act 604 of FIG. 6), neither the IgK nor the IgL prediction may be selected as output 737 for the first filtration.

For the second filtration step, the coverages for each set of cell population estimates 722, 724 are compared to one another and to a coverage factor. In this example, the coverage of the set of IgK cell population estimates 722 exceeds the coverage of the set of IgL cell population estimates 724 by a factor of 100. Since this exceeds the coverage factor of 50, this satisfies the first criteria (e.g., act 608 of FIG. 6), and the IgK prediction 732 is selected as output 738 for the second filtration.

Based on the outputs 737, 738 of the filtration steps, a final output 739 is selected. Since, neither of the results was selected as output 737 for the first filtration step, but the IgK prediction 732 was selected as the output 738 for the second filtration step, the IgK prediction 732 is selected as the final output 739.

The example in FIG. 7C includes a set of IgK cell population estimates 742 and a set of IgL cell population estimates 744. For the purpose of this example, let the fraction of sequence reads associated with the largest cell population estimate of the set of IgK cell population estimates 742 be 0.7 and the fraction of sequence reads associated with the largest cell population estimate of the set of IgL cell population estimates 744 be 0.3. Further, the coverages, as shown in FIG. 7C, are 20,000 for both sets of cell population estimates 742, 744.

At stage 710, features for each set of cell population estimates 742, 744 are processed using the trained machine learning model 711 to determine whether the largest cell population estimate includes malignant cells of a first type. IgK prediction 752 corresponds to determining that the largest cell population estimate of the set of IgK cell population estimates 742 includes malignant cells of the first type. IgL prediction 753 corresponds to determining that the largest cell population estimate of the set of IgL cell population estimates 744 includes malignant cells of the first type.

At stage 715, the sizes of the largest cell population estimates and the coverages for each set of cell population estimates 742, 744 are used as input 756 to the two filtration steps, described herein including at least with respect to FIGS. 4-6.

For the first filtration step, the sizes of the largest cell population estimates may be compared to the maximum and minimum size thresholds. In this example, 0.7, the size of the largest cell population estimate of the set of IgK cell population estimates 742, exceeds the minimum size threshold of 0.2. Additionally, 0.3, the size of the largest cell population estimate of the set of IgL cell population estimates 744, is less than the maximum size threshold of 0.5. Since this satisfies the first criteria (e.g., act 602 of FIG. 6), the IgK prediction 752 is selected as the output 757 for the first filtration.

For the second filtration step, the coverages for each set of cell population estimates 742, 744 are compared to one another and to a coverage factor. In this example, neither coverage exceeds the other by the coverage factor. As such, neither the first nor the second criteria are satisfied (e.g., acts 608 and 609 of FIG. 6), so neither the IgK nor the IgL prediction 752, 753 is selected as the output 758 for the second filtration.

Based on the outputs 757, 758 of the filtration steps, a final output 759 is selected. Since the IgK prediction 752 was selected as the output 757 for the first filtration, but neither result was selected as the output 758 for the second filtration, the IgK prediction is selected as the final output 759.

The example in FIG. 7D includes a set of IgK cell population estimates 762 and a set of IgL cell population estimates 764. For the purpose of this example, let the fraction of sequence reads associated with the largest cell population estimate of the set of IgK cell population estimates 762 be 0.7 and the fraction of sequence reads associated with the largest cell population estimate of the set of IgL cell population estimates 764 be 0.3. Further, the coverages, as shown in FIG. 7D, are 200 and 20,000 for the set of IgK cell population estimates 762 and the set of IgL cell population estimates 764, respectively.

At stage 710, features for each set of cell population estimates 762, 764 are processed using the trained machine learning model 711 to determine whether the largest cell population estimate includes malignant cells of a first type. IgK prediction 772 corresponds to determining that the largest cell population estimate of the set of IgK cell population estimates 762 includes malignant cells of the first type. IgL prediction 773 corresponds to determining that the largest cell population estimate of the set of IgL cell population estimates 764 includes malignant cells of the first type.

At stage 715, the sizes of the largest cell population estimates and the coverages for each set of cell population estimates 762, 764 are used as input 776 to the two filtration steps, described herein including at least with respect to FIGS. 4-6.

For the first filtration step, the sizes of the largest cell population estimates are compared to the maximum and minimum size thresholds. In this example, 0.7, the size of the largest cell population estimate of the set of IgK cell population estimates 762, exceeds the minimum size threshold of 0.2. Additionally, 0.3, the size of the largest cell population estimate of the set of IgL cell population estimates 764, is less than the maximum size threshold of 0.5. Since this satisfies the first criteria (e.g., act 602 of FIG. 6), the IgK prediction 772 is selected as output 777 for the first filtration.

For the second filtration step, the coverages for each set of cell population estimates 762, 764 are compared to one another and to a coverage factor. In this example, the coverage of the set of IgL cell population estimates 764 exceeds the coverage of the set of IgK cell population estimates 762 by a factor of 100. Since this exceeds the coverage factor of 50, this satisfies the second criteria (e.g., act 609 of FIG. 6), and the IgL prediction 773 is selected as output 778 for the second filtration.

Based on the outputs 777, 778 of the filtration steps, a final output 779 is selected. Since the IgK prediction 772 was selected as the output 777 and the IgL prediction 773 was selected as the output 778, neither the IgK nor the IgL prediction is selected as the final output 779. As such, the sample used to generate the cell population estimates may be considered an outlier.

The example in FIG. 7E includes a set of IgK cell population estimates 782 and a set of IgL cell population estimates 784. For the purpose of this example, let the fraction of sequence reads associated with the largest cell population estimate of the set of IgK cell population estimates 782 be 0.5 and the fraction of sequence reads associated with the largest cell population estimate of the set of IgL cell population estimates 784 be 0.5. Further, the coverages, as shown in FIG. 7E, are 20,000 for both the set of IgK cell population estimates 782 and the set of IgL cell population estimates 784.

At stage 710, features for each set of cell population estimates 782, 784 are processed using the trained machine learning model 711 to determine whether the largest cell population estimate includes malignant cells of a first type. IgK prediction 792 corresponds to determining that the largest cell population estimate of the set of IgK cell population estimates 782 includes malignant cells of the first type. IgL prediction 793 corresponds to determining that the largest cell population estimate of the set of IgL cell population estimates 784 includes malignant cells of the first type.

At stage 715, the fractions of the largest cell population estimates and the coverages for each set of cell population estimates 782, 784 are used as input 796 to the two filtration steps, described herein including at least with respect to FIGS. 4-6.

For the first filtration step, the sizes of the largest cell population estimates are compared to the maximum and minimum size thresholds. In this example, 0.5, the size of the largest cell population estimate of the set of IgK cell population estimates 782, exceeds the minimum size threshold of 0.2. However, 0.5, the size of the largest cell population estimate of the set of IgL cell population estimates 784, exceeds the maximum size threshold of 0.5. Similarly, the size of the largest cell population estimate of the set of IgL cell population estimates 784 exceeds the minimum size threshold of 0.2, but the size of the largest cell population estimate of the set of IgK cell population estimates 782 is exceeds the maximum size threshold of 0.5. Since this satisfies neither the first criteria (e.g., act 602 of FIG. 6) nor the second criteria (e.g., act 604 of FIG. 6), neither the IgK nor the IgL prediction are selected as output 797 for the first filtration.

For the second filtration step, the coverages for each set of cell population estimates 782, 784 are compared to one another and to a coverage factor. In this example, neither coverage exceeds the other by the coverage factor. As such, neither the first nor the second criteria are satisfied (e.g., acts 608 and 609 of FIG. 6), so neither the IgK nor the IgL prediction is selected as the output 798 for the second filtration.

Based on the outputs 797, 798 of the filtration steps, a final output 799 is selected. Since neither the IgK nor the IgL prediction was selected for either of the outputs 797, 798 of the filtration steps, neither the IgK nor the IgL prediction is selected as the final output 799.

FIG. 8 is a flowchart of an illustrative process 800 for training a machine learning model to identify malignant cell populations in biological samples, in accordance with some embodiments of the technology described herein.

In some embodiments, machine learning model 802 may include, but is not limited to a: a naïve Bayes classifier, a support vector machine (SVM) classifier, a decision tree classifier, a random forest classifier, a neural network classifier, a non-linear regression classifier, a logistic regression classifier, or an ensemble classifier (for example, an Adaboost classifier).

In some embodiments, the techniques include obtaining sequencing data 804 from one or more biological samples. In some embodiments, the sequencing data 804 may include training data, test data, and experimental data for training machine learning model 802. In some embodiments, each of the datasets may include data from biological samples that are healthy and/or biological samples that are cancerous, as described in the section “Biological Samples.” For example, the training datasets and testing datasets may each include datasets from a healthy donor and datasets from tissue diagnosed with lymphoma, while the experimental datasets may only include datasets from tissue diagnosed with lymphoma. In some embodiments oversampling methods, such as Synthetic Minority Oversampling Technique (SMOTE), may be applied to the training datasets.

The sequencing data 804 may be processed to identify a plurality of cell population estimates, which may include one or more sets of cell population estimates. In some embodiments, each set of cell population estimates may be processed to identify features at step 806. For example, the features may include, for each set of cell population estimates, the size of the largest cell population estimate and the ratio between the sizes of the largest and second largest cell population estimates. In some embodiments, only one set of cell population estimates (e.g., associated with one receptor chain) is processed to identify features at step 806.

In some embodiments, it may be desirable to select training and/or testing datasets with certain characteristics. For example, it may be desirable to select training datasets that have sequencing data that is well covered to produce a robust machine learning model. A well-covered dataset may have a larger average coverage relative to the other available datasets. As another example, it may be desirable to select testing datasets that have a high clonality.

In some embodiments, information pertaining to the sequencing data 804, such as coverage, may be used to filter the input to the machine learning algorithm. Samples with low average coverage may contain potentially incorrect information and may be filtered out at a low coverage filtration step 808. In some embodiments, it may be desirable to filter out sequencing data that has a coverage below a given threshold, as described above.

In some embodiments, the features identified at act 806 for each set of the cell population estimates are processed, at act 810, using the machine learning model 802 to determine whether the largest cell population estimate of each set includes malignant cells of the first type (e.g., act 810). In some embodiments, an output is obtained for each set of estimate cell populations.

In some embodiments, a training stage may include a hyperparameter grid search 810 and cross-validation 812. The training stage may train the machine learning model 802 on the features identified from the training datasets at act 806. The hyperparameter grid search 810 may be used to obtain the hyperparameters for the machine learning model 802. For example, hyperparameters may include learning rate and a number of estimators.

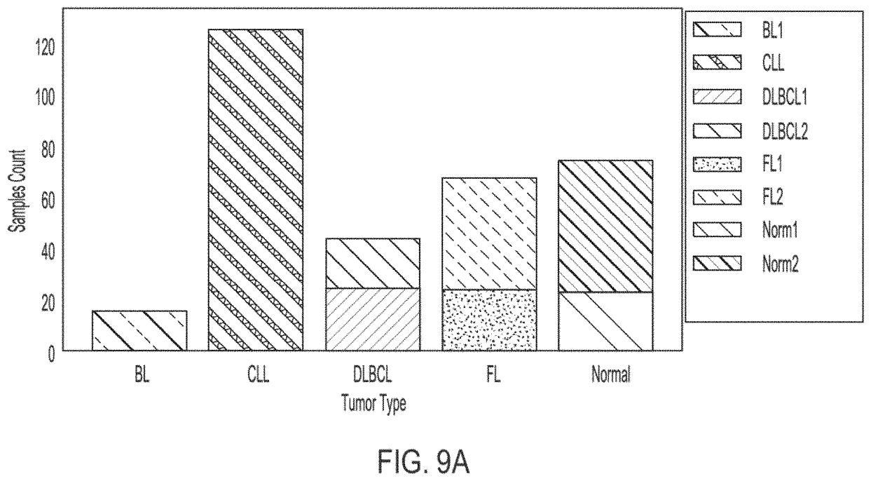

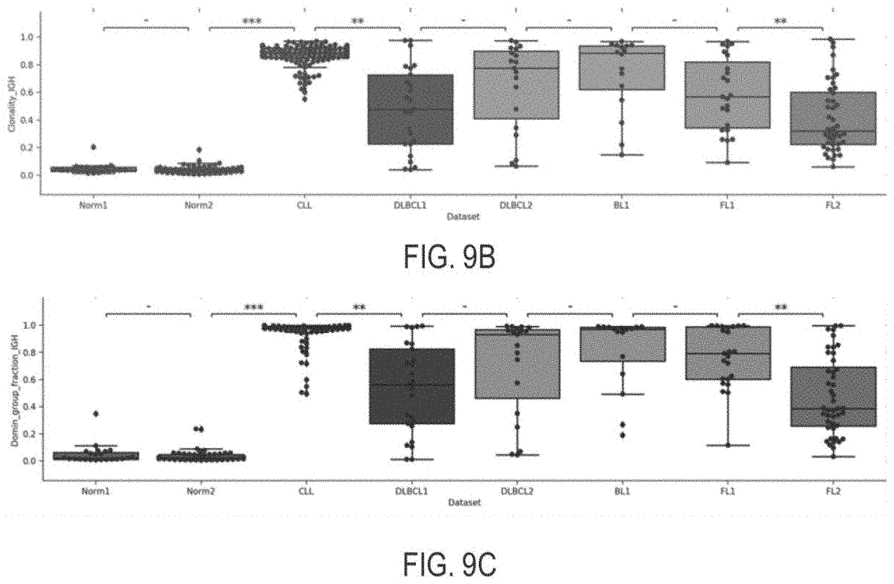

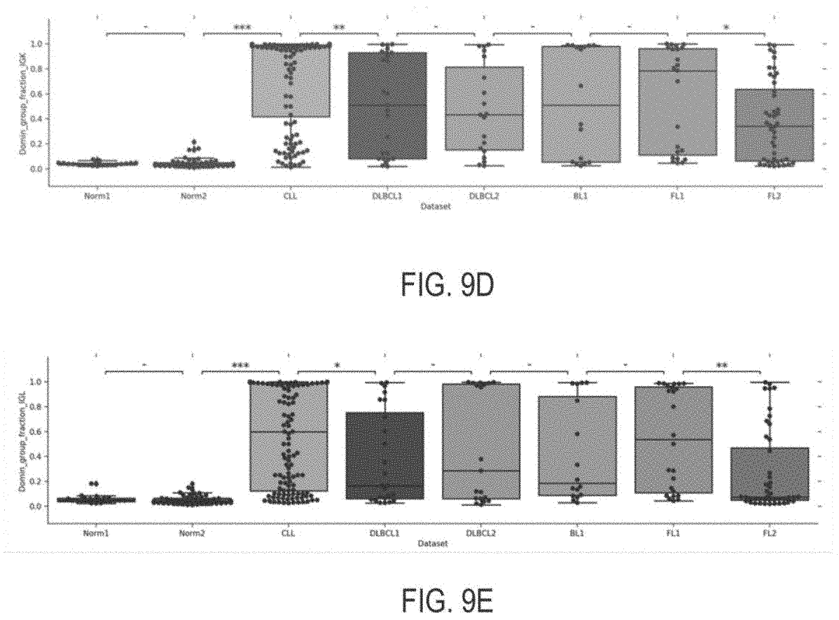

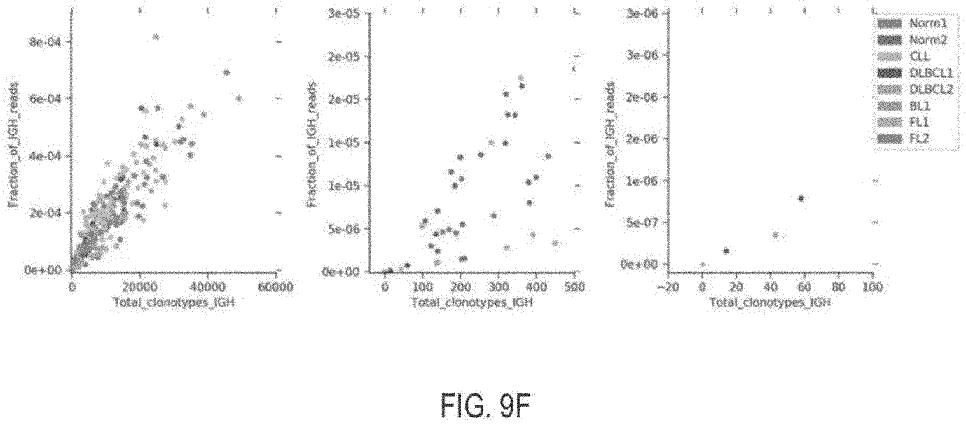

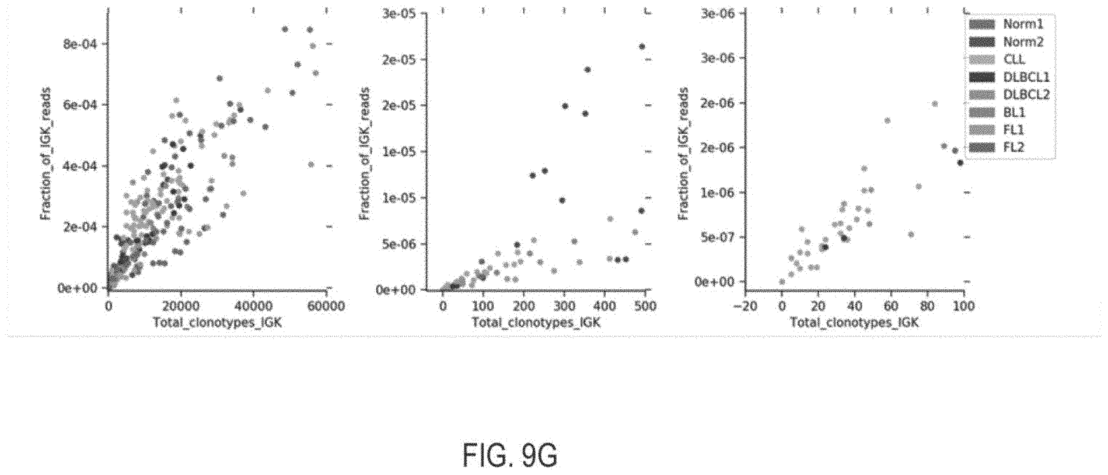

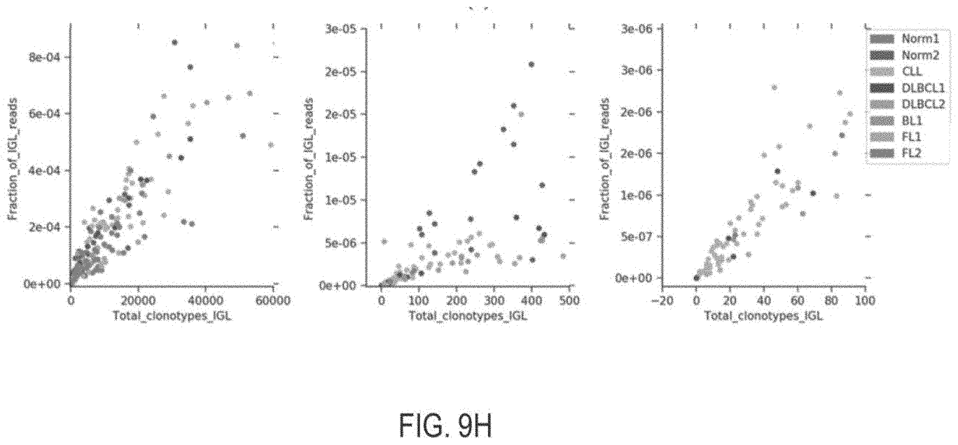

In some embodiments, it may be beneficial to perform a hyperparameter grid search 810 for more than one type of machine learning model 802 and to evaluate each of the machine learning models during cross-validation 812. One of the machine learning models may then be selected based on the metrics that result from cross-validation 812. For example, in some embodiments, such metrics may include area under the receiver operating characteristic curve (AUC ROC), precision, recall, accuracy, and Fi score. Below, FIGS. 9A-G provide non-limiting, illustrative examples of the described training and machine learning model selection methods.

In some embodiments, once the machine learning model 802 is trained, the process 800 proceeds to the testing stage 814. A hyperparameter grid search and cross-validation may also be performed on the testing data during stage 814. Metrics and predictions 816 may be output at the testing stage 814 as a result of cross-validation and testing, respectively. In some embodiments, machine learning model 802 may be applied to the testing datasets several times to ensure that the model is stable. In some embodiments, all the potential machine learning models may be tested or only the selected machine learning model may be tested.

After the testing stage 814, process 800 proceeds to the experimental stage 818. At this stage, the machine learning model 802 may be trained to identify malignant cell populations in any biological sample, as described herein. In some embodiments, experimental datasets may be input as sequencing data 804 to yield predictions 820. The predictions 820 may be the result of determining whether a largest cell population estimate includes malignant cells of a first type.

In some embodiments, a separate machine learning model may be trained, using process 800, for sequencing data 804 for each type of receptor chain for each cell type. For example, a machine learning model may be trained on sequencing data associated with the IgH receptor chain and a separate machine learning model may be trained on sequencing data associated with TRB. In other embodiments, a machine learning model trained on sequencing data for one type of immunoglobulin may also be used to make predictions on sequencing data for another type of immunoglobulin. For example, a machine learning model trained on sequencing data associated with IgH may be used to make predictions based on sequencing data associated with the immunoglobulin light chains, IgL and IgK.

### EXAMPLE 1

### Example Datasets for Training and Testing a Machine Learning Model for Identifying Malignant Cell Populations

FIGS. 9A-H are graphs and plots that depict descriptive statistics for example datasets used for training and testing the example machine learning models, in accordance with some embodiments of the technology described herein. The datasets were obtained from the Genotype-Tissue Expression (GTEx) Portal, International Cancer Genome Consortium (ICGC), and other publicly accessible sources. Table 1 lists the label and source for each dataset.

TABLE 1
Dataset sources.

| Dataset | Dataset |
|---|---|
| FL1 | SRP056293 |
| FL2 | MALY-DE from ICGC: |
| BL1 | |
| DLBCL1 | |
| DLBCL2 | |
| CLL | CLLE_ES from ICGC |
| Norml and | GSE45982 |
| Norm2- | GSE58335 |
| combination | GSE111405 |
| from a few | GSE57944 |
| datasets | GTEx |
| | Phs000424.v6.p1 from dbGAP |
| | GSE84022 |
| | GSE63816 |
| | GSE61410 |
| | GSE112057 |
| | GSE90081 |
| | GSE60424 |
| | GSE120795 |
| | GSE43603 |
| TCR Dataset | SRP044708 |

Experiments were undertaken using the example datasets to train, test, and experiment on a machine learning model, as described with respect to the above embodiments. Table 2 lists each of the datasets used for such training, testing, and experimenting. The Norm 2 dataset was selected for training over the Norm 1 dataset because of its greater average coverage. FIG. 9A is a bar plot summarizing the types of tumors in the sample datasets summarized in Table 2.

TABLE 2
Datasets for testing, training, and experimenting
on a machine learning model for
identifying malignant cell populations in B cell samples.

| Dataset | | | Number of | Average | |
|---|---|---|---|---|---|
| Name | Disease | Status | Samples | Coverage | Stage |
| CLL | Chronic | Tumor | 128 | 10475 | Test |
| | lymphocytic | | | | |
| | leukemia | | | | |
| DLBCL | Diffuse | Tumor | 25 | 6198 | Experi- |
| 1 | large B-cell | | | | mental |
| | lymphoma | | | | |
| DLBCL | Diffuse | Tumor | 19 | 15018 | Experi- |
| 2 | large B-cell | | | | mental |
| | lymphoma | | | | |
| BL 1 | B-cell lymphoma | Tumor | 16 | 9507 | Experi- |
| | | | | | mental |
| FL 1 | Follicular | Tumor | 24 | 6752 | Train |
| | lymphoma | | | | |
| FL 2 | Follicular | Tumor | 44 | 21769 | Experi- |
| | lymphoma | | | | mental |
| Norm 1 | Normal cells | Normal | 22 | 3729 | Test |
| Norm 2 | Normal cells | Normal | 53 | 10475 | Train |

FIG. 9B is a plot indicating the IgH clonality distribution for each of the example datasets in Table 2. Clonality is in indication of whether there is one dominant (e.g., large) cell population in a sample, or whether there are several small cell populations in a sample. If the clonality is high, this indicates that there is likely one dominant clone. If the clonality is low, this indicates that there are many small, difference cell populations in a sample. The CLL dataset was chosen as a test dataset due to its high clonality.

FIGS. 9C-E are plots indicating the sizes of the largest initial cell population estimates (e.g., dominant group fraction) in different samples, where the cell population estimates were generated using IgH, IgK, and IgL receptor chains, respectively. Each datapoint represents the largest cell population in a sample of the dataset. Unlike the normal datasets (e.g., Norm1 and Norm2), samples with large cell population estimates are present in the cancer samples. A large number of such groups in the FL1 and CLL datasets indicates the purity of the data.

FIGS. 9F-H are plots showing the proportion of the largest initial cell population estimate in a sample compared to the number of initial cell populations estimates identified for the sample, where the initial cell population estimates were generated using IgH, IgK, and IgL receptor chains, respectively.

The horizontal axis shows the total number of initial cell population estimates (e.g., clonotypes). The vertical axis shows fraction of sequence reads for the largest initial cell population estimate (e.g., dominant clonotype) in the sample. A large number of samples having a largest initial cell population estimate of the same or of a similar size indicates a purity of the data (e.g., the FL1 dataset).

### EXAMPLE 2

### Visual Representation of Grouping Cells in Cell Populations

Experiments were undertaken to generate an exemplary report for identifying a plurality of cell population estimates from sequencing data. The report may be generated as a result of processing the sequencing data as described herein including at least with respect to FIGS. 2 and 3A-B.

FIG. 10A is a visual of an example report and user interface generated during the identification of B cell population estimates. The visual shows an overview of some of the statistics made available for the sequencing data. The statistics may include immunoglobulin heavy and/or light chain clonality and diversity, a total number of CDR3 identified, diagnoses associated with specific CDR3 present in the sample, the B cell CDR3 region of the dominant cell population in the sample, information regarding BCR antigens, IGHV mutation status for the BCR, explicit or not BCR CDR3 for B cell lymphoma, and characteristics for B cell lymphoma.

Clonality and diversity provide an indication of whether there is one large cell population that is dominant in the biological sample (e.g., high clonality and low diversity), or whether there are several, small, different cell populations in the biological sample.

The characteristics for B cell lymphoma may include fraction of the dominant cell population, light chain of the dominant cell population, CDR3 sequences for the immunoglobulin heavy and light chains for the dominant cell populations, and stereotyped V or J gene usage.

Further, the user interface includes an option for a user to select a receptor chain. In some embodiments, choosing a receptor chain may be equivalent to choosing a set of the plurality of cell population estimates, as discussed herein including at least with respect to FIGS. 2 and 3A-B. As a result of choosing a receptor chain, the report displays a visual of how sequence reads associated with identical CDR3 regions are combined to obtain initial cell population estimates, and how the initial cell population estimates are combined to form a set of cell population estimates. The visual includes a map and an associated chart for displaying the initial cell populations and set of cell population estimates. In the present example, these are labelled “Clonal Composition” and “Clonal Composition Map,” respectively.

FIG. 10B is an example of a map for displaying a set of cell population estimates when there is a large initial cell population estimate (e.g., a large number of sequence reads with identical CDR3 regions). The initial cell population estimates are represented by the nodes and the cell population estimates are represented by the initial cell population estimates that are connected to one another by edges. The size of the node corresponds to the size of the initial cell population estimate, or the fraction of sequence reads that cover its particular CDR3. The initial cell population estimates connected to one another represent somatic hypermutation results (e.g., the CDR3 of each of the initial cell population estimates was derived from the same CDR3.) In this example, a single initial cell population estimate makes up 91% of the sequence reads in the sample. Additionally, six other initial cell population estimates are connected to the large initial cell population estimate, representing a cell population estimate that makes up 99.91% of the sample. The bolded outline encompassing this cell population estimate may indicate that it has been identified to be malignant using the machine learning techniques described herein.

FIG. 10C is an example of a map for displaying the initial cell population estimates and cell population estimates when there is no dominant initial cell population estimate. As shown, the size of each of the initial cell population estimates varies slightly, the largest initial cell population making up 0.52% of the sample. This example also shows cell population estimates, represented by the nodes connected by edges.

FIG. 10D is an example representation of selecting a set of cell population estimates associated with IgH and, as a result, displaying initial cell population estimates which are connected to form the set cell population estimates associated with IgH. This display further shows a chart that breaks down the initial cell population estimates by size (e.g., large, doubleton, singleton) and displays the percentage of each size within the sample.

FIG. 10E is an example representation of selecting a set of cell population estimates associated with IgL and, as a result, displaying the initial cell population estimates, which are connected to form the set of cell population estimates associated with IgL.

FIG. 10F is an example representation of selecting a set of cell population estimates associated with IgK and, as a result, displaying the initial cell population estimates, which are connected to form the set of cell population estimates associated with IgK.

FIG. 10G is an example representation of selecting a set of initial cell population estimates associated with TRA and, as a result, displaying the initial cell population estimates associated with TRA.

FIG. 10H is an example representation of selecting a set of cell population estimates associated with IgH and, as a result, displaying the initial cell population estimates, which are connected to form the set of cell population estimates associated with IgH.

The visuals of FIG. 10H correspond to the display of FIG. 10I, which lists each of the initial cell population estimates, differing by their unique CDR3 regions. The display shows, for each of the initial cell population estimates, the amino acid sequence and nucleotide sequence shared by the initial cell population estimate, lists the V(D)J segments, identifies the percentage of reads for the initial cell population estimate compared to all of the initial cell population estimates (e.g., fraction), identifies the cell population estimate the initial cell population estimate belongs to, notes the number of reads covering the same CDR3 sequence, and lists the isotype. As an example, FIG. 10H shows an initial cell population estimate represented by the largest node on the map. Further, the map shows that the fraction of this estimate is 22%. This initial cell population estimate is connected to 25 other initial cell population estimates, as shown by the connecting edges and legend, making up cell population estimate 1, which has a proportion of 26.5%. The first entry of FIG. 10I includes all of the information about the largest initial cell population estimate, and subsequent entries include information about initial cell population estimates that also belong to cell population estimate 1. The sequencing data for each of the initial cell population estimates in cell population estimate 1 is also given. As shown, the amino acid and nucleotide sequences are not identical for the first two initial cell population estimates in cell population estimate 1 (e.g., first two entries), but they are very similar. The sequence reads were clustered to identify this similarity and combine them into the same cell population estimate.

FIG. 10J is a screenshot of an example report indicating information about cell population estimates for a biological sample. The report shows example statistics associated with the sequencing data, including specific details for each type of receptor chain. In this example, only the IgK receptor chain may be explicit due to its high clonality (e.g., one dominant cell population estimate (compared to the low clonality of the IgL receptor chain). Therefore, this sample may not require filtering stages for identifying an output associated with either the IgK or IgL receptor chain.

FIG. 10K corresponds to the example report in FIG. 10J and includes maps that show the set of cell population estimates associated with IgH and the set of cell population estimates associated with IgK. The red outline encompassing the largest cell population estimate in each map indicates that these cell population estimates have been identifies as containing malignant cells using the machine learning techniques described herein.

### EXAMPLE 3

### Training and Selecting a Machine Learning Model

FIGS. 11A-D depict graphs illustrating the decision boundaries of different example machine learning models when selected by accuracy, precision, recall, and F₁, respectively. For each figure, the columns represent different machine learning models (e.g., SVM, Random Forest, AdaBoost, and Naïve Bayes) and the rows represent different coverage thresholds applied to the data (e.g., coverage thresholds of 0, 40, and 100). For each subplot, the horizontal axis is the dominant group fraction (e.g., size of largest cell populations) and the vertical axis is the ratio of the dominant group fraction and second group fraction (e.g., ratio of sizes of largest and second largest cell populations). The FL1 and Norm 2 datasets are used for training and cross validating the algorithm. The coverage of samples was used as a weight in the classification.

Table 3 shows the average metrics, corresponding to FIGS. 11A-D, on the cross-validation datasets, while Table 4 shows the average metrics of the classifiers on test datasets, CLL and Norm 1. For both tables, the first column contains the metric by which the classifier was selected during the hyperparameter grid search, the second column contains the average coverage threshold, the third column contains the classifier names, and the remaining columns contain cross-validation metrics. The classifier used for this particular model (e.g., Naïve Bayes classifier selected for accuracy with a coverage threshold of 40), is shaded in grey. Although the selected model does not show maximum values, it returns the optimal decision boundary (shown in FIG. 11A) and corresponds to a biological understanding of the problem.

TABLE 3
Model evaluation on cross-validation datasets

| Metric | Threshold | Classifier | Accuracy | Precision | Recall | F1 | AUC ROC |
|---|---|---|---|---|---|---|---|
| Accuracy | 0 | SVM | 98.72 | 98.33 | 100.0 | 99.12 | 96.35 |
| | | Random Forest | 97.44 | 98.33 | 98.15 | 99.14 | 97.04 |
| | | AdaBoost | 98.72 | 98.33 | 100.0 | 99.12 | 97.5 |
| | | Naïve Bayes | 96.15 | 98.15 | 96.3 | 97.17 | 97.27 |
| | 40 | SVM | 98.72 | 98.33 | 100.0 | 99.12 | 96.35 |
| | | Random Forest | 97.44 | 98.33 | 98.15 | 98.14 | 97.04 |
| | | AdaBoost | 98.72 | 98.33 | 100.0 | 99.12 | 97.5 |
| | | Naïve Bayes | 96.15 | 98.15 | 96.30 | 97.17 | 97.04 |
| | 100 | SVM | 98.61 | 98.33 | 100.0 | 99.12 | 94.71 |
| | | Random Forest | 98.61 | 98.33 | 100.0 | 99.12 | 95.9 |
| | | AdaBoost | 98.61 | 98.33 | 100.0 | 99.12 | 96.8 |
| | | Naïve Bayes | 95.94 | 98.15 | 96.3 | 97.17 | 96.2 |
| Precision | 0 | SVM | 98.72 | 98.33 | 100.0 | 99.12 | 98.15 |
| | | Random Forest | 98.72 | 98.33 | 100.0 | 99.12 | 97.04 |
| | | AdaBoost | 97.44 | 98.33 | 98.15 | 98.14 | 97.5 |
| | | Naïve Bayes | 97.44 | 96.67 | 100.0 | 98.25 | 96.35 |
| | 40 | SVM | 98.72 | 98.33 | 100.0 | 99.12 | 96.35 |
| | | Random Forest | 98.72 | 98.33 | 100.0 | 99.12 | 97.04 |
| | | AdaBoost | 98.72 | 98.33 | 100.0 | 99.12 | 97.5 |
| | | Naïve Bayes | 97.44 | 96.67 | 100.0 | 98.25 | 96.35 |
| | 100 | SVM | 98.61 | 98.33 | 100.0 | 99.12 | 99.49 |
| | | Random Forest | 98.61 | 98.33 | 100.0 | 99.12 | 95.6 |
| | | AdaBoost | 98.61 | 98.33 | 100.0 | 99.12 | 96.8 |
| | | Naïve Bayes | 97.22 | 96.48 | 100.0 | 98.14 | 94.71 |
| Recall | 0 | SVM | 98.72 | 98.33 | 100.0 | 99.12 | 96.35 |
| | | Random Forest | 97.44 | 98.33 | 98.15 | 98.14 | 97.04 |
| | | AdaBoost | 97.44 | 98.33 | 98.15 | 98.14 | 97.5 |
| | | Naïve Bayes | 96.15 | 98.15 | 96.3 | 97.17 | 97.27 |
| | 40 | SVM | 98.72 | 98.33 | 100..0 | 99.12 | 96.35 |
| | | Random Forest | 98.72 | 98.33 | 100.0 | 99.12 | 97.5 |
| | | AdaBoost | 98.72 | 98.33 | 100.0 | 99.12 | 97.5 |
| | | Naïve Bayes | 96.15 | 98.15 | 96.3 | 97.17 | 97.27 |
| | 100 | SVM | 98.61 | 98.33 | 100.0 | 99.12 | 94.71 |
| | | Random Forest | 97.33 | 98.33 | 98.15 | 98.14 | 95.01 |
| | | AdaBoost | 98.61 | 98.33 | 100.0 | 99.12 | 96.8 |
| | | Naïve Bayes | 49.25 | 61.11 | 31.48 | 39.83 | 94.7 |
| F1 | 0 | SVM | 97.33 | 96.48 | 100.0 | 98.14 | 95.9 |
| | | Random Forest | 98.72 | 98.33 | 100.0 | 99.12 | 97.5 |
| | | AdaBoost | 98.72 | 98.33 | 100.0 | 99.12 | 97.5 |
| | | Naïve Bayes | 96.15 | 98.15 | 96.3 | 97.17 | 97.04 |
| | 40 | SVM | 98.72 | 98.33 | 100.0 | 99.12 | 96.35 |
| | | Random Forest | 98.72 | 98.33 | 100.0 | 99.12 | 97.5 |
| | | AdaBoost | 97.44 | 98.33 | 98.15 | 98.14 | 97.5 |
| | | Naïve Bayes | 96.15 | 98.15 | 96.3 | 97.17 | 97.04 |
| | 100 | SVM | 98.61 | 98.33 | 100.0 | 99.12 | 94.71 |
| | | Random Forest | 97.33 | 98.33 | 98.15 | 98.14 | 96.2 |
| | | AdaBoost | 98.61 | 98.33 | 100.0 | 99.12 | 96.8 |
| | | Naïve Bayes | 97.22 | 96.48 | 100.0 | 98.14 | 94.71 |

TABLE 4
Model evaluation on test datasets.

| Metric | Threshold | Classifier | Accuracy | Precision | Recall | F1 | AUC ROC |
|---|---|---|---|---|---|---|---|
| Accuracy | 0 | SVM | 100.0 | 100.0 | 100.0 | 100.0 | 99.49 |
| | | Random Forest | 99.34 | 100.0 | 95.65 | 97.76 | 99.49 |
| | | AdaBoost | 100.0 | 100.0 | 100.0 | 100.0 | 99.49 |
| | | Naïve Bayes | 90.73 | 100.0 | 39.13 | 56.25 | 99.49 |
| | 40 | SVM | 100.0 | 100.0 | 100.0 | 100.0 | 99.49 |
| | | Random Forest | 99.34 | 100.0 | 95.65 | 97.76 | 99.49 |
| | | AdaBoost | 99.87 | 100.0 | 99.13 | 99.56 | 99.49 |
| | | Naïve Bayes | 94.7 | 100.0 | 65.22 | 78.95 | 99.49 |
| | 100 | SVM | 100.0 | 100.0 | 100.0 | 100.0 | 99.49 |
| | | Random Forest | 100.0 | 100.0 | 100.0 | 100.0 | 99.49 |
| | | AdaBoost | 99.74 | 100.0 | 98.26 | 99.11 | 99.49 |
| | | Naïve Bayes | 90.73 | 100.0 | 39.13 | 56.25 | 99.49 |
| Precision | 0 | SVM | 100.0 | 100.0 | 100.0 | 100.0 | 99.49 |
| | | Random Forest | 100.0 | 100.0 | 100.0 | 100.0 | 99.49 |
| | | AdaBoost | 99.87 | 100.0 | 99.13 | 99.56 | 99.49 |
| | | Naïve Bayes | 97.35 | 85.19 | 100.0 | 92.0 | 99.49 |
| | 40 | SVM | 100.0 | 100.0 | 100.0 | 100.0 | 99.49 |
| | | Random Forest | 99.87 | 100.0 | 99.13 | 99.56 | 99.49 |
| | | AdaBoost | 99.74 | 100.0 | 98.26 | 99.11 | 99.49 |
| | | Naïve Bayes | 97.35 | 85.19 | 100.0 | 92.0 | 99.49 |
| | 100 | SVM | 100.0 | 100.0 | 100.0 | 100.0 | 99.49 |
| | | Random Forest | 100.0 | 100.0 | 100.0 | 100.0 | 99.49 |
| | | AdaBoost | 99.74 | 100.0 | 98.26 | 99.11 | 99.49 |
| | | Naïve Bayes | 91.39 | 100.0 | 43.38 | 60.61 | 99.45 |
| Recall | 0 | SVM | 100.0 | 100.0 | 100.0 | 100.0 | 99.49 |
| | | Random Forest | 99.6 | 100.0 | 97.39 | 98.65 | 99.49 |
| | | AdaBoost | 99.87 | 100.0 | 99.13 | 99.56 | 99.49 |
| | | Naïve Bayes | 85.43 | 100.0 | 4.35 | 8.33 | 99.49 |
| | 40 | SVM | 100.0 | 100.0 | 100.0 | 100.0 | 99.49 |
| | | Random Forest | 99.34 | 100.0 | 95.65 | 97.78 | 99.49 |
| | | AdaBoost | 99.6 | 100.0 | 97.39 | 98.6 | 99.49 |
| | | Naïve Bayes | 90.73 | 100.0 | 39.13 | 56.25 | 99.49 |
| | 100 | SVM | 100.0 | 100.0 | 100.0 | 100.0 | 99.49 |
| | | Random Forest | 99.47 | 100.0 | 96.52 | 98.2 | 99.49 |
| | | AdaBoost | 99.87 | 100.0 | 99.13 | 99.56 | 99.49 |
| | | Naïve Bayes | 93.38 | 100.0 | 56.52 | 72.22 | 99.49 |
| F1 | 0 | SVM | 100.0 | 100.0 | 100.0 | 100.0 | 99.49 |
| | | Random Forest | 99.34 | 100.0 | 95.65 | 97.78 | 99.49 |
| | | AdaBoost | 99.87 | 100.0 | 99.13 | 99.56 | 99.49 |
| | | Naïve Bayes | 85.43 | 100.0 | 4.35 | 8.33 | 99.49 |
| | 40 | SVM | 100.0 | 100.0 | 100.0 | 100.0 | 99.49 |
| | | Random Forest | 99.34 | 100.0 | 95.65 | 97.78 | 99.49 |
| | | AdaBoost | 100.0 | 100.0 | 100.0 | 100.0 | 99.49 |
| | | Naïve Bayes | 96.03 | 100.0 | 73.91 | 85.0 | 99.49 |
| | 100 | SVM | 100.0 | 100.0 | 100.0 | 100.0 | 99.49 |
| | | Random Forest | 99.87 | 100.0 | 99.13 | 99.56 | 99.46 |
| | | AdaBoost | 99.74 | 100.0 | 98.26 | 99.11 | 99.49 |
| | | Naïve Bayes | 91.39 | 100.0 | 43.48 | 60.61 | 99.45 |

FIG. 11E is an example calibration plot for the different example machine learning models (e.g., SVM, Random Forest, AdaBoost, and Naïve Bayes) with hyperparameters selected by accuracy and an average coverage threshold of 40.

FIG. 11F is an example probability distribution for the different example machine learning models (e.g., SVM, Random Forest, AdaBoost, and Naïve Bayes) with hyperparameters selected by accuracy and an average coverage threshold of 40.

FIG. 11G is an example averaged receiver operating characteristic (ROC) curve for a selected machine learning model (e.g., Naive Bayes) with hyperparameters chosen by accuracy and a coverage threshold of 40.

### EXAMPLE 4

### Example Results of Classifying Immunoglobulin Light Chains

FIG. 12 shows example results of identifying an output prediction (e.g., an IgK or IgL prediction) when a biological sample includes B cells with both types of light chain receptors. “Normal” datapoints correspond to a biological sample that results in only one set of cell population estimates for a light chain receptor. The remaining datapoints indicate an output selected based on the filtration steps described with respect to FIGS. 4-7. The “IgK” datapoints indicate that the result associated with IgK was selected as output, the “IgL” datapoints indicate that the result associated with IgL was selected as output, and the “Outlier” datapoints indicate that neither result was selected for that particular sample.

### EXAMPLE 5

### Testing and Using a Selected Machine Learning Model to Identify Malignant Cell Populations

FIGS. 13A-D depict example predictions as a result of implementing technique 100 for identifying malignant cell populations.

FIG. 13A is an example graph depicting the predictions of a Naive Bayes classifier used to process sequencing data from a test dataset associated with cell population estimates for cells associated with IgH. As shown, the horizontal axis indicates the IgH dominant group fraction. In some embodiments, the IgH dominant group fraction is indicative of the size of the largest cell population estimate identified for a sample. The vertical axis indicates the ratio between the IgH dominant group fractions of a largest and second largest cell population estimates identified for the sample. In some embodiments, this ratio is indicative of a ratio between the sizes of the largest cell population and the second largest cell population of a sample. The shading is indicative of the decision boundaries of the classifier, determining the likelihood that the cell population is one of two classes. In some embodiments, the two classes are malignant or normal. In this example, the data points are classified as malignant if they are within the grey region and they are classified as normal if they are within the red region. In some embodiments, the data points may be representative of the largest cell population estimate identified for the sample.

FIG. 13B is an example chart illustrating how the Naive Bayes classifier used to process sequencing data in an experimental dataset associated with cell population estimates for cells associated with IgH chains, as described in relation to FIG. 13A.

FIG. 13C predicted cell population estimates in FIG. 13B for each of the biological samples from the experimental dataset. As shown, the horizontal axis indicates the class, or biological sample, from which the sequencing data was collected. The vertical axis indicates the normalized number of cell populations, ranging from 0 (none) to 1.0 (all cell populations within the sample). As indicated by the legend, the shading on the bars is indicative of how the cell population estimates identified for each sample were classified by the techniques described at least in part by FIG. 2. The cell population estimates were classified as malignant (or tumor), normal, or as having a coverage value that did not exceed the coverage threshold. In some embodiments, cell population estimates with a coverage value that does not exceed the coverage threshold (e.g., coverage factor) are not included in the machine learning classification techniques described herein.

FIG. 13D is another example graph depicting the predictions of a Naïve Bayes classifier used to process sequencing data for cell population estimates for cells associated with TCR chains, as described in relation to FIG. 13A.

FIG. 13E shows predictions with the Naive Bayes classifier using test datasets for B cell samples. The graph plots the IgH dominant group counts against IgK dominant group counts for each sample.

FIG. 13F shows predictions with the Naive Bayes classifier using experimental datasets for B cell samples. The graph plots the IgH dominant group counts against IgL dominant group counts for each sample.

Aspects of the disclosure provide computer implemented methods for identifying one or more malignant cell populations within a biological sample.

In some embodiments, a software program may provide a user with a visual representation presenting information relates to cell populations within a biological sample (e.g., identification of cell populations and subsequent classification as malignant or normal). Such a software program may execute in any suitable computing environment including, but not limited to, a cloud-computing environment, a device co-located with a user (e.g., the user's laptop, desktop, smartphone, etc.), one or more devices remote from the user (e.g., one or more servers), etc.

For example, in some embodiments, the techniques described herein may be implemented in the illustrative environment 1400 shown in FIG. 14. As shown in FIG. 14, within illustrative environment 1400, one or more biological samples of a patient 1402 may be provided to a laboratory 1404. Laboratory 1404 may process the biological sample(s) to obtain sequencing data (e.g., transcriptome, exome, and/or genome sequencing data) and provide it via network 1408, to at least one database 1406 that stores information about patient 1402.

Network 1408 may be a wide area network (e.g., the Internet), a local area network (e.g., a corporate Intranet), and/or any other suitable type of network. Any of the devices shown in FIG. 14 may connect to the network 1408 using one or more wired links, one or more wireless links, and/or any suitable combination thereof.

In the illustrated embodiment of FIG. 14, the at least one database 1406 may store sequencing data for the patient, sequencing data for the patient, medical history data for the patient, test result data for the patient, and/or any other suitable information about the patient 1402. Examples of stored test result data for the patient include biopsy test results, imaging test results (e.g., MRI results), and blood test results. The information stored in at least one database 1406 may be stored in any suitable format and/or using any suitable data structure(s), as aspects of the technology described herein are not limited in this respect. The at least one database 1406 may store data in any suitable way (e.g., one or more databases, one or more files). The at least one database 1406 may be a single database or multiple databases.

As shown in FIG. 14, illustrative environment 1400 includes one or more external databases 1416, which may store information for patients other than patient 1402. For example, external databases 1416 may store sequencing data (of any suitable type) for one or more patients, medical history data for one or more patients, test result data (e.g., imaging results, biopsy results, blood test results) for one or more patients, demographic and/or biographic information for one or more patients, and/or any other suitable type of information. In some embodiments, external database(s) 1416 may store information available in one or more publicly accessible databases such as TCGA (The Cancer Genome Atlas), one or more databases of clinical trial information, and/or one or more databases maintained by commercial sequencing suppliers. The external database(s) 1416 may store such information in any suitable way using any suitable hardware, as aspects of the technology described herein are not limited in this respect.

In some embodiments, the at least one database 1406 and the external database(s) 1416 may be the same database, may be part of the same database system, or may be physically co-located, as aspects of the technology described herein are not limited in this respect.

In some embodiments, information stored in patient information database 1406 and/or in external database(s) 1416 may be used to perform any of the techniques described herein related to determining a therapy score and/or impact score indicative of a patient's response to a therapy. For example, the information stored in the database(s) 1406 and/or 1416 may be accessed, via network 1408, by software executing on server(s) 1410 to perform any one or more of the techniques described herein in connection with FIG. 2.

For example, in some embodiments, server(s) 1410 may access information stored in database(s) 1406 and/or 1416 and use this information to perform process 200, described with reference to FIG. 2, for identifying malignant cell population(s) based on sequencing data obtained from biological samples.

In some embodiments, server(s) 1410 may include multiple computing devices. When server(s) 1410 include multiple computing devices, the device(s) may be physically co-located (e.g., in a single room) or distributed across multi-physical locations. In some embodiments, server(s) 1410 may be part of a cloud computing infrastructure. In some embodiments, one or more server(s) 1410 may be co-located in a facility operated by an entity (e.g., a hospital, research institution) with which doctor 1414 is affiliated. In such embodiments, it may be easier to allow server(s) 1410 to access private medical data for the patient 1402.

As shown in FIG. 14, in some embodiments, the results of the analysis performed by server(s) 1410 may be provided to doctor 1414 through a computing device 1414 (which may be a portable computing device, such as a laptop or smartphone, or a fixed computing device such as a desktop computer). The results may be provided in a written report, an e-mail, a graphical user interface, and/or any other suitable way. It should be appreciated that although in the embodiment of FIG. 14, the results are provided to a doctor, in other embodiments, the results of the analysis may be provided to patient 1402 or a caretaker of patient 1402, a healthcare provider such as a nurse, or a person involved with a clinical trial.

In some embodiments, the results may be part of a graphical user interface (GUI) presented to the doctor 1414 via the computing device 1412. In some embodiments, the GUI may be presented to the user as part of a webpage displayed by a web browser executing on the computing device 1412. In some embodiments, the GUI may be presented to the user using an application program (different from a web-browser) executing on the computing device 1412. For example, in some embodiments, the computing device 1412 may be a mobile device (e.g., a smartphone) and the GUI may be presented to the user via an application program (e.g., “an app”) executing on the mobile device.

### Biological Samples

Any of the methods, systems, or other claimed elements may use or be used to analyze a biological sample from a subject. In some embodiments, a biological sample is obtained from a subject having, suspected of having cancer, or at risk of having cancer. The biological sample may be any type of biological sample including, for example, a biological sample of a bodily fluid (e.g., blood, urine or cerebrospinal fluid), one or more cells (e.g., from a scraping or brushing such as a cheek swab or tracheal brushing), a piece of tissue (cheek tissue, muscle tissue, lung tissue, heart tissue, brain tissue, skin tissue, or blood), or some or all of an organ (e.g., brain, lung, liver, bladder, kidney, pancreas, intestines, or muscle), or other types of biological samples (e.g., feces or hair).

In some embodiments, the biological sample is a sample of a tumor from a subject. In some embodiments, the biological sample is a sample of blood from a subject. In some embodiments, the biological sample is a sample of tissue from a subject.

A sample of a tumor, in some embodiments, refers to a sample comprising cells from a tumor. In some embodiments, the sample of the tumor comprises cells from a benign tumor, e.g., non-cancerous cells. In some embodiments, the sample of the tumor comprises cells from a premalignant tumor, e.g., precancerous cells. In some embodiments, the sample of the tumor comprises cells from a malignant tumor, e.g., cancerous cells.

Examples of tumors include, but are not limited to, adenomas, fibromas, hemangiomas, lipomas, cervical dysplasia, metaplasia of the lung, leukoplakia, carcinoma, sarcoma, germ cell tumors, and blastoma.

A sample of blood, in some embodiments, refers to a sample comprising cells, e.g., cells from a blood sample. In some embodiments, the sample of blood comprises non-cancerous cells. In some embodiments, the sample of blood comprises precancerous cells. In some embodiments, the sample of blood comprises cancerous cells. In some embodiments, the sample of blood comprises a combination of non-cancerous, precancerous, and/or cancerous cells. In some embodiments, the sample of blood comprises blood cells. In some embodiments, the sample of blood comprises red blood cells. In some embodiments, the sample of blood comprises white blood cells. In some embodiments, the sample of blood comprises platelets. Examples of cancerous blood cells include, but are not limited to, leukemia, lymphoma, and myeloma. In some embodiments, a sample of blood is collected to obtain the cell-free nucleic acid (e.g., cell-free DNA) in the blood.

A sample of blood may be a sample of whole blood or a sample of fractionated blood. In some embodiments, the sample of blood comprises whole blood. In some embodiments, the sample of blood comprises fractionated blood. In some embodiments, the sample of blood comprises buffy coat. In some embodiments, the sample of blood comprises serum. In some embodiments, the sample of blood comprises plasma. In some embodiments, the sample of blood comprises a blood clot.

A sample of a tissue, in some embodiments, refers to a sample comprising cells from a tissue. In some embodiments, the sample of the tissue comprises non-cancerous cells from a tissue. In some embodiments, the sample of the tissue comprises precancerous cells from a tissue. In some embodiments, the sample of the tumor comprises cancerous cells from a tissue. In some embodiments, the sample of the tumor comprises a combination of non-cancerous cells, precancerous cells, and/or cancerous cells from a tissue.

Methods of the present disclosure encompass a variety of tissue including organ tissue or non-organ tissue, including but not limited to, muscle tissue, brain tissue, lung tissue, liver tissue, epithelial tissue, connective tissue, and nervous tissue. In some embodiments, the tissue may be normal tissue or it may be diseased tissue or it may be tissue suspected of being diseased. In some embodiments, the tissue may be sectioned tissue or whole intact tissue. In some embodiments, the tissue may be animal tissue or human tissue. Animal tissue includes, but is not limited to, tissues obtained from rodents (e.g., rats or mice), primates (e.g., monkeys), dogs, cats, and farm animals.

The biological sample may be from any source in the subject's body including, but not limited to, any fluid [such as blood (e.g., whole blood, blood serum, or blood plasma), saliva, tears, synovial fluid, cerebrospinal fluid, pleural fluid, pericardial fluid, ascitic fluid, and/or urine], hair, skin (including portions of the epidermis, dermis, and/or hypodermis), oropharynx, laryngopharynx, esophagus, stomach, bronchus, salivary gland, tongue, oral cavity, nasal cavity, vaginal cavity, anal cavity, bone, bone marrow, brain, thymus, spleen, small intestine, appendix, colon, rectum, anus, liver, biliary tract, pancreas, kidney, ureter, bladder, urethra, uterus, vagina, vulva, ovary, cervix, scrotum, penis, prostate, testicle, seminal vesicles, and/or any type of tissue (e.g., muscle tissue, epithelial tissue, connective tissue, or nervous tissue). Any of the biological samples described herein may be obtained from the subject using any known technique. See, for example, the following publications on collecting, processing, and storing biological samples, each of which are incorporated by reference herein in its entirety: Biospecimens and biorepositories: from afterthought to science by Vaught et al. (Cancer Epidemiol Biomarkers Prev. 2012 February; 21(2):253-5), and Biological sample collection, processing, storage and information management by Vaught and Henderson (IARC Sci Publ. 2011;(163):23-42).

In some embodiments, the biological sample may be obtained from a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy).

In some embodiments, one or more than one cell (i.e., a cell biological sample) may be obtained from a subject using a scrape or brush method. The cell biological sample may be obtained from any area in or from the body of a subject including, for example, from one or more of the following areas: the cervix, esophagus, stomach, bronchus, or oral cavity. In some embodiments, one or more than one piece of tissue (e.g., a tissue biopsy) from a subject may be used. In certain embodiments, the tissue biopsy may comprise one or more than one (e.g., 2, 3, 4, 5, 6, 7, 8, 9, 10, or more than 10) biological samples from one or more tumors or tissues known or suspected of having cancerous cells.

Any of the biological samples from a subject described herein may be stored using any method that preserves stability of the biological sample. In some embodiments, preserving the stability of the biological sample means inhibiting components (e.g., DNA, RNA, protein, or tissue structure or morphology) of the biological sample from degrading until they are measured so that when measured, the measurements represent the state of the sample at the time of obtaining it from the subject. In some embodiments, a biological sample is stored in a composition that is able to penetrate the same and protect components (e.g., DNA, RNA, protein, or tissue structure or morphology) of the biological sample from degrading. As used herein, degradation is the transformation of a component from one from to another such that the first form is no longer detected at the same level as before degradation.

In some embodiments, a biological sample (e.g., tissue sample) is fixed. As used herein, a “fixed” sample relates to a sample that has been treated with one or more agents or processes in order to prevent or reduce decay or degradation, such as autolysis or putrefaction, of the sample. Examples of fixative processes include but are not limited to heat fixation, immersion fixation, and perfusion. In some embodiments a fixed sample is treated with one or more fixative agents. Examples of fixative agents include but are not limited to cross-linking agents (e.g., aldehydes, such as formaldehyde, formalin, glutaraldehyde, etc.), precipitating agents (e.g., alcohols, such as ethanol, methanol, acetone, xylene, etc.), mercurials (e.g., B-5, Zenker's fixative, etc.), picrates, and Hepes-glutamic acid buffer-mediated organic solvent protection effect (HOPE) fixative. In some embodiments, a biological sample (e.g., tissue sample) is treated with a cross-linking agent. In some embodiments, the cross-linking agent comprises formalin. In some embodiments, a formalin-fixed biological sample is embedded in a solid substrate, for example parrafin wax. In some embodiments, the biological sample is a formalin-fixed paraffin-embedded (FFPE) sample. Methods of preparing FFPE samples are known, for example as described by Li et al. JCO Precis Oncol. 2018; 2: P0.17.00091.

In some embodiments, the biological sample is stored using cryopreservation. Non-limiting examples of cryopreservation include, but are not limited to, step-down freezing, blast freezing, direct plunge freezing, snap freezing, slow freezing using a programmable freezer, and vitrification. In some embodiments, the biological sample is stored using lyophilisation. In some embodiments, a biological sample is placed into a container that already contains a preservant (e.g., RNALater to preserve RNA) and then frozen (e.g., by snap-freezing), after the collection of the biological sample from the subject. In some embodiments, such storage in frozen state is done immediately after collection of the biological sample. In some embodiments, a biological sample may be kept at either room temperature or 4° C. for some time (e.g., up to an hour, up to 8 h, or up to 1 day, or a few days) in a preservant or in a buffer without a preservant, before being frozen.

Non-limiting examples of preservants include formalin solutions, formaldehyde solutions, RNALater or other equivalent solutions, TriZol or other equivalent solutions, DNA/RNA Shield or other equivalent solutions, EDTA (e.g., Buffer AE (10 mM Tris.Cl; 0.5 mM EDTA, pH 9.0)) and other coagulants, and Acids Citrate Dextronse (e.g., for blood specimens).

In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

Any of the biological samples from a subject described herein may be stored under any condition that preserves stability of the biological sample. In some embodiments, the biological sample is stored at a temperature that preserves stability of the biological sample. In some embodiments, the sample is stored at room temperature (e.g., 25° C.). In some embodiments, the sample is stored under refrigeration (e.g., 4° C.). In some embodiments, the sample is stored under freezing conditions (e.g., −20° C.). In some embodiments, the sample is stored under ultralow temperature conditions (e.g., −50° C. to −800° C.). In some embodiments, the sample is stored under liquid nitrogen (e.g., −1700° C.). In some embodiments, a biological sample is stored at −60° C. to −8° C. (e.g., −70° C.) for up to 5 years (e.g., up to 1 month, up to 2 months, up to 3 months, up to 4 months, up to 5 months, up to 6 months, up to 7 months, up to 8 months, up to 9 months, up to 10 months, up to 11 months, up to 1 year, up to 2 years, up to 3 years, up to 4 years, or up to 5 years). In some embodiments, a biological sample is stored as described by any of the methods described herein for up to 20 years (e.g., up to 5 years, up to 10 years, up to 15 years, or up to 20 years).

Methods of the present disclosure encompass obtaining one or more biological samples from a subject for analysis. In some embodiments, one biological sample is collected from a subject for analysis. In some embodiments, more than one (e.g., 2, 3, 4, 5, 6, 7, 8, 9, 10, 11, 12, 13, 14, 15, 16, 17, 18, 19, 20, or more) biological samples are collected from a subject for analysis. In some embodiments, one biological sample from a subject will be analyzed. In some embodiments, more than one (e.g., 2, 3, 4, 5, 6, 7, 8, 9, 10, 11, 12, 13, 14, 15, 16, 17, 18, 19, 20, or more) biological samples may be analyzed. If more than one biological sample from a subject is analyzed, the biological samples may be procured at the same time (e.g., more than one biological sample may be taken in the same procedure), or the biological samples may be taken at different times (e.g., during a different procedure including a procedure 1, 2, 3, 4, 5, 6, 7, 8, 9, 10 days; 1, 2, 3, 4, 5, 6, 7, 8, 9, 10 weeks; 1, 2, 3, 4, 5, 6, 7, 8, 9, 10 months, 1, 2, 3, 4, 5, 6, 7, 8, 9, 10 years, or 1, 2, 3, 4, 5, 6, 7, 8, 9, 10 decades after a first procedure).

A second or subsequent biological sample may be taken or obtained from the same region (e.g., from the same tumor or area of tissue) or a different region (including, e.g., a different tumor). A second or subsequent biological sample may be taken or obtained from the subject after one or more treatments and may be taken from the same region or a different region. As a non-limiting example, the second or subsequent biological sample may be useful in determining whether the cancer in each biological sample has different characteristics (e.g., in the case of biological samples taken from two physically separate tumors in a patient) or whether the cancer has responded to one or more treatments (e.g., in the case of two or more biological samples from the same tumor or different tumors prior to and subsequent to a treatment). In some embodiments, each of the at least one biological sample is a bodily fluid sample, a cell sample, or a tissue biopsy sample.

In some embodiments, one or more biological specimens are combined (e.g., placed in the same container for preservation) before further processing. For example, a first sample of a first tumor obtained from a subject may be combined with a second sample of a second tumor from the subject, wherein the first and second tumors may or may not be the same tumor. In some embodiments, a first tumor and a second tumor are similar but not the same (e.g., two tumors in the brain of a subject). In some embodiments, a first biological sample and a second biological sample from a subject are sample of different types of tumors (e.g., a tumor in muscle tissue and brain tissue).

In some embodiments, a sample from which RNA and/or DNA is extracted (e.g., a sample of tumor, or a blood sample) is sufficiently large such that at least 2 μg (e.g., at least 2 μg, at least 2.5 μg, at least 3 μg, at least 3.5 μg or more) of RNA can be extracted from it. In some embodiments, the sample from which RNA and/or DNA is extracted can be peripheral blood mononuclear cells (PBMCs). In some embodiments, the sample from which RNA and/or DNA is extracted can be any type of cell suspension. In some embodiments, a sample from which RNA and/or DNA is extracted (e.g., a sample of tumor, or a blood sample) is sufficiently large such that at least 1.8 μg of RNA can be extracted from it. In some embodiments, at least 50 mg (e.g., at least 1 mg, at least 2 mg, at least 3 mg, at least 4 mg, at least 5 mg, at least 10 mg, at least 12 mg, at least 15 mg, at least 18 mg, at least 20 mg, at least 22 mg, at least 25 mg, at least 30 mg, at least 35 mg, at least 40 mg, at least 45 mg, or at least 50 mg) of tissue sample is collected from which RNA and/or DNA is extracted. In some embodiments, at least 20 mg of tissue sample is collected from which RNA and/or DNA is extracted. In some embodiments, at least 30 mg of tissue sample is collected. In some embodiments, at least 10-50 mg (e.g., 10-50 mg, 10-15 mg, 10-30 mg, 10-40 mg, 20-30 mg, 20-40 mg, 20-50 mg, or 30-50 mg) of tissue sample is collected from which RNA and/or DNA is extracted. In some embodiments, at least 30 mg of tissue sample is collected. In some embodiments, at least 20-30 mg of tissue sample is collected from which RNA and/or DNA is extracted. In some embodiments, a sample from which RNA and/or DNA is extracted (e.g., a sample of tumor, or a blood sample) is sufficiently large such that at least 0.2 μg (e.g., at least 200 ng, at least 300 ng, at least 400 ng, at least 500 ng, at least 600 ng, at least 700 ng, at least 800 ng, at least 900 ng, at least 1 μg, at least 1.1 μg, at least 1.2 μg, at least 1.3 μg, at least 1.4 μg, at least 1.5 μg, at least 1.6 μg, at least 1.7 μg, at least 1.8 μg, at least 1.9 μg, or at least 2 μg) of RNA can be extracted from it. In some embodiments, a sample from which RNA and/or DNA is extracted (e.g., a sample of tumor, or a blood sample) is sufficiently large such that at least 0.1 μg (e.g., at least 100 ng, at least 200 ng, at least 300 ng, at least 400 ng, at least 500 ng, at least 600 ng, at least 700 ng, at least 800 ng, at least 900 ng, at least 1 μg, at least 1.1 μg, at least 1.2 μg, at least 1.3 μg, at least 1.4 μg, at least 1.5 μg, at least 1.6 μg, at least 1.7 μg, at least 1.8 μg, at least 1.9 μg, or at least 2 μg) of RNA can be extracted from it.

### Subjects

Aspects of this disclosure relate to a biological sample that has been obtained from a subject. In some embodiments, a subject is a mammal (e.g., a human, a mouse, a cat, a dog, a horse, a hamster, a cow, a pig, or other domesticated animal). In some embodiments, a subject is a human. In some embodiments, a subject is an adult human (e.g., of 18 years of age or older). In some embodiments, a subject is a child (e.g., less than 18 years of age). In some embodiments, a human subject is one who has or has been diagnosed with at least one form of cancer. In some embodiments, a cancer from which a subject suffers is a carcinoma, a sarcoma, a myeloma, a leukemia, a lymphoma, or a mixed type of cancer that comprises more than one of a carcinoma, a sarcoma, a myeloma, a leukemia, and a lymphoma. Carcinoma refers to a malignant neoplasm of epithelial origin or cancer of the internal or external lining of the body. Sarcoma refers to cancer that originates in supportive and connective tissues such as bones, tendons, cartilage, muscle, and fat. Myeloma is cancer that originates in the plasma cells of bone marrow. Leukemias (“liquid cancers” or “blood cancers”) are cancers of the bone marrow (the site of blood cell production). Lymphomas develop in the glands or nodes of the lymphatic system, a network of vessels, nodes, and organs (specifically the spleen, tonsils, and thymus) that purify bodily fluids and produce infection-fighting white blood cells, or lymphocytes. Non-limiting examples of a mixed type of cancer include adenosquamous carcinoma, mixed mesodermal tumor, carcinosarcoma, and teratocarcinoma. In some embodiments, a subject has a tumor. A tumor may be benign or malignant. In some embodiments, a cancer is any one of the following: skin cancer, lung cancer, breast cancer, prostate cancer, colon cancer, rectal cancer, cervical cancer, and cancer of the uterus. In some embodiments, a subject is at risk for developing cancer, e.g., because the subject has one or more genetic risk factors, or has been exposed to or is being exposed to one or more carcinogens (e.g., cigarette smoke, or chewing tobacco).

### Sequencing Data

As aspects of the disclosure relate to methods for identifying malignant cell populations in a biological sample using sequencing data obtained from the biological sample from a subject.

The sequencing data used in methods described herein may be derived from sequencing data obtained from the biological sample. In some embodiments, the sequencing data may include RNA sequencing data, which may be termed “RNA expression data.” In some embodiments, the sequencing data may include any type of data from which RNA expression data may derived. The sequencing data (e.g., RNA expression data) may be acquired using any method known in the art including, but not limited to: whole transcriptome, sequencing, total RNA sequencing, mRNA sequencing, targeted RNA sequencing, RNA exome capture sequencing, next generation sequencing, and/or deep RNA sequencing. In some embodiments, sequencing data (e.g., RNA expression data) may be obtained by processing microarray data.

In some embodiments, in which the sequencing data does not itself include RNA expression data, the sequencing data may be processed to produce RNA expression data. In some such embodiments, the sequencing data may be processed by one or more bioinformatics methods or software tools, for example RNA sequence quantification tools (e.g., Kallisto) and genome annotation tools (e.g., Gencode v23), in order to produce the RNA expression data. The Kallisto software is described in Nicolas L Bray, Harold Pimentel, Pall Melsted and Lior Pachter, Near-optimal probabilistic RNA-seq quantification, Nature Biotechnology 34, 525-527 (2016), doi:10.1038/nbt.3519, which is incorporated by reference in its entirety herein.

In some embodiments, microarray expression data may be processed using a bioinformatics R package, such as “affy” or “limma”, in order to produce RNA expression data. The “affy” software is described in Bioinformatics. 2004 Feb. 12; 20(3):307-15. doi:

10.1093/bioinformatics/btg405. “affy-analysis of Affymetrix GeneChip data at the probe level” by Laurent Gautier 1, Leslie Cope, Benjamin M Bolstad, Rafael A Irizarry PMID: 14960456 DOI: 10.1093/bioinformatics/btg405, which is incorporated by reference herein in its entirety. The “limma” software is described in Ritchie M E, Phipson B, Wu D, Hu Y, Law CW, Shi W, Smyth GK “limma powers differential expression analyses for RNA-sequencing and microarray studies.” Nucleic Acids Res. 2015 Apr. 20; 43(7):e47. 20. https://doi.org/10.1093/nar/gkv007 PMID: 25605792, PMCID: PMC4402510, which is incorporated by reference herein its entirety.

In some embodiments, the sequencing data comprises more than 5 kilobases (kb). For example, the sequencing data may comprise at least 10 kb, at least 100 kb, at least 500 kb, at least 1 megabase (Mb), at least 10 Mb, at least 100 Mb, at least 500 Mb, at least 1 gigabase (Gb), at least 10 Gb, at least 100 Gb, at least 500 Gb, between 10 kB and 100 Mb, between 1 Mb and 500 Gb, between 100 kb and 10 Gb, or any other suitable range within these ranges.

In some embodiments, the RNA expression data is acquired through bulk RNA sequencing. Bulk RNA sequencing may include obtaining expression levels for each gene across RNA extracted from a large population of input cells (e.g., a mixture of different cell types.) In some embodiments, the RNA expression data may be acquired through single cell sequencing (e.g., scRNA-seq). Single cell sequencing may include sequencing individual cells.

In some embodiments, bulk sequencing data comprises at least 1 million reads, at least 5 million reads, at least 10 million reads, at least 20 million reads, at least 50 million reads, or at least 100 million reads. In some embodiments, bulk sequencing data comprises between 1 million reads and 5 million reads, 3 million reads and 10 million reads, 5 million reads and 20 million reads, 10 million reads and 50 million reads, 30 million reads and 100 million reads, or 1 million reads and 100 million reads (or any number of reads including, and between).

In some embodiments, the RNA expression data comprises next-generation sequencing (NGS) data. In some embodiments, the RNA expression data comprises microarray data.

In some embodiments, sequencing data includes RNA expression data (e.g., indicating expression levels) for a plurality of genes, which may be used for any of the methods or compositions described herein. The number of genes which may be examined may be up to and inclusive of all the genes of the subject. In some embodiments, expression levels may be determined for all of the genes of a subject. As a non-limiting example, four or more, five or more, six or more, seven or more, eight or more, nine or more, ten or more, eleven or more, twelve or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 35 or more, 40 or more, 50 or more, 60 or more, 70 or more, 80 or more, 90 or more, 100 or more, 125 or more, 150 or more, 175 or more, 200 or more, 225 or more, 250 or more, 275 or more, or 300 or more genes may be used for any evaluation described herein.

In some embodiments, RNA expression data is obtained by accessing the RNA expression data from at least one computer storage medium on which the RNA expression data is stored. Additionally or alternatively, in some embodiments, RNA expression data may be received from one or more sources via a communication network of any suitable type. For example, in some embodiment, the RNA expression data may be received from a server (e.g., a SFTP server, or Illumina B aseSpace).

In some embodiments, the RNA expression data obtained may be in any suitable format, as aspects of the technology described herein are not limited in this respect. For example, in some embodiments, the RNA expression data may be obtained in a text-based file (e.g., in a FASTQ, FASTA, BAM, or SAM format). In some embodiments, a file in which sequencing data is stored may contains quality scores of the sequencing data. In some embodiments, a file in which sequencing data is stored may contain sequence identifier information.

The sequencing data, in some embodiments, includes gene expression levels. Gene expression levels may be detected by detecting a product of gene expression such as mRNA and/or protein. In some embodiments, gene expression levels are determined by detecting a level of a mRNA in a sample. As used herein, the terms “determining” or “detecting” may include assessing the presence, absence, quantity and/or amount (which can be an effective amount) of a substance within a sample, including the derivation of qualitative or quantitative concentration levels of such substances, or otherwise evaluating the values and/or categorization of such substances in a sample from a subject.

In some embodiments, sequencing data may be generated using a nucleic acid from a sample from a subject. In some embodiments, the sequencing data may indicate a nucleotide sequence of DNA and/or RNA from a previously obtained biological sample of a subject having, suspected of having, or at risk of having a disease. In some embodiments, the nucleic acid is deoxyribonucleic acid (DNA). In some embodiments, the nucleic acid is prepared such that the whole genome is present in the nucleic acid. In some embodiment, the nucleic acid is prepared such that fragmented DNA and/or RNA is present in the nucleic acid. In some embodiments, the nucleic acid is processed such that only the protein coding regions of the genome remain (e.g., exomes). When nucleic acids are prepared such that only the exomes are sequenced, it is referred to as whole exome sequencing (WES). A variety of methods are known in the art to isolate the exomes for sequencing, for example, solution-based isolation wherein tagged probes are used to hybridize the targeted regions (e.g., exomes) which can then be further separated from the other regions (e.g., unbound oligonucleotides). These tagged fragments can then be prepared and sequenced.

DNA sequencing data, in some embodiments, refers to a level of DNA (e.g., copy number of a chromosome, gene, or other genomic region) in a sample from a subject. The level of DNA in a sample from a subject having cancer may be elevated compared to the level of DNA in a sample from a subject not having cancer, e.g., a gene duplication in a cancer patient's sample. The level of DNA in a sample from a subject having cancer may be reduced compared to the level of DNA in a sample from a subject not having cancer, e.g., a gene deletion in a cancer patient's sample.

DNA sequencing data, in some embodiments, refers to data (e.g., sequencing data) obtained by processing a biological sample (e.g., DNA (e.g., coding or non-coding genomic DNA) present in a biological sample) using a sequencing apparatus. DNA that is present in a sample may or may not be transcribed, but it may be sequenced using DNA sequencing platforms. Such data may be useful, in some embodiments, to determine whether the patient has one or more mutations associated with a particular cancer.

Sequencing data may be generated by the nucleic acid sequencing protocol (e.g., the series of nucleotides in a nucleic acid molecule identified by any suitable generation of sequencing (Sanger sequencing, Illumina®, next-generation sequencing (NGS) etc.), as well as information contained therein (e.g., information indicative of source, tissue type, etc.) which may also be considered information that can be inferred or determined from the sequencing data. For example, in some embodiments RNA sequencing data may be analyzed to determine whether the nucleic acid was primarily polyadenylated or not.

DNA sequencing data may be acquired using any method known in the art including any known method of DNA sequencing. For example, DNA sequencing may be used to identify one or more mutations in the DNA of a subject. Any technique used in the art to sequence DNA may be used with the methods and compositions described herein. As a set of non-limiting examples, the DNA may be sequenced through single-molecule real-time sequencing, ion torrent sequencing, pyrosequencing, sequencing by synthesis, sequencing by ligation (SOLiD sequencing), nanopore sequencing, or Sanger sequencing (chain termination sequencing).

Protein expression data may be acquired using any method known in the art including, but not limited to: N-terminal amino acid analysis, C-terminal amino acid analysis, Edman degradation (including though use of a machine such as a protein sequenator), or mass spectrometry.

In some embodiments, sequencing data may include raw DNA or RNA sequencing data, DNA exome sequencing data (e.g., from whole exome sequencing (WES), DNA genome sequencing data (e.g., from whole genome sequencing (WGS)), RNA sequencing data, gene sequencing data, bias-corrected gene sequencing data, or any other suitable type of sequencing data comprising data obtained from a sequencing platform and/or comprising data derived from data obtained from a sequencing platform.

In some embodiments, the sequencing platform may be a next generation sequencing platform (e.g., Illumina®, Roche®, Ion Torrent®, etc.), or any high-throughput or massively parallel sequencing platform. In some embodiments, these methods may be automated, in some embodiments, there may be manual intervention. In some embodiments, the sequencing data may be the result of non-next generation sequencing (e.g., Sanger sequencing). In some embodiments, the sample preparation may be according to manufacturer's protocols. In some embodiments, the sample preparation may be custom made protocols, or other protocols which are for research, diagnostic, prognostic, and/or clinical purposes. In some embodiments, the protocols may be experimental.

### Alignment and Annotation

In some embodiments, a method to process sequencing data (e.g., data obtained from RNA sequencing (also referred to herein as RNA-seq data)) comprises aligning and annotating genes in the RNA sequencing data with known sequences of the human genome to obtain annotated RNA sequencing data.

In some embodiments, alignment of sequencing data comprises aligning the data to a known assembled genome for a particular species of subject (e.g., the genome of a human) or to a transcriptome database. Various sequence alignment software is available and can be used to align data to an assembled genome or a transcriptome database. Non-limiting examples of alignment software includes short (unspliced) aligners (e.g., BLAT; BFAST, Bowtie, Burrows-Wheeler Aligner, Short Oligonucleotide Analysis package, or Mosaik), spliced aligners, aligners based on known splice junctions (e.g., Errange, IsoformEx, or Splice Seq), or de novo splice aligner (e.g., ABMapper, BBMap, CRAC, or HiSAT). In some embodiments, any suitable tool can be used for aligning and annotating data. For example, Kallisto (github.com/pachterlab/kallisto) is used to align and annotate data. In some embodiments, a known genome is referred to as a reference genome. A reference genome (also known as a reference assembly) is a digital nucleic acid sequence database, assembled as a representative example of a species' set of genes. In some embodiments, human and mouse reference genomes used in any one of the methods described herein are maintained and improved by the Genome Reference Consortium (GRC). Non-limiting examples of human reference releases are GRCh38, GRCh37, NCBI Build 36.1, NCBI Build 35, and NCBI Build 34. A non-limiting example of transcriptome databases include Transcriptome Shotgun Assembly (TSA).

In some embodiments, annotating sequencing data comprises identifying the locations of genes and/or coding regions in the data to be processed by comparing it to assembled genomes or transcriptome databases. Non-limiting examples of data sources for annotation include GENCODE (www<dot>gencodegenes<dot>org), RefSeq (see e.g., www<dot>ncbi<dot>nlm<dot>nih<dot>gov/refseq/), and Ensembl. In some embodiments, annotating genes in RNA sequencing data is based on a GENCODE database (e.g., GENCODE V23 annotation; www<dot>gencodegenes<dot>org).

Consea et al. (A survey of best practices for RNA-seq data analysis; Genome Biology201617:13) provides best practices for analyzing RNA-seq data, which are applicable to any one of the methods described herein and is incorporated by reference in its entirety herein. Pereira and Rueda (RNA-seq: From reads to counts, Course Materials, Cambridge, U.K.: Cambridge Institute (2015)) also describe methods for analyzing sequencing data, which are applicable to any one of the methods described herein, and is incorporated by reference herein in its entirety.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

An illustrative implementation of a computer system 1500 that may be used in connection with any of the embodiments of the technology described herein (e.g., such as the methods of FIGS. 2 and 4) is shown in FIG. 15. The computer system 1500 includes one or more processors 1510 and one or more articles of manufacture that comprise non-transitory computer-readable storage media (e.g., memory 1520 and one or more non-volatile storage media 1530). The processor 1510 may control writing data to and reading data from the memory 1520 and the non-volatile storage device 1530 in any suitable manner, as the aspects of the technology described herein are not limited in this respect. To perform any of the functionality described herein, the processor 1510 may execute one or more processor-executable instructions stored in one or more non-transitory computer-readable storage media (e.g., the memory 1520), which may serve as non-transitory computer-readable storage media storing processor-executable instructions for execution by the processor 1510.

Computing device 1500 may also include a network input/output (I/O) interface 1540 via which the computing device may communicate with other computing devices (e.g., over a network), and may also include one or more user I/O interfaces 1550, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel. It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

1. A method, comprising:
using at least one computer hardware processor to perform:
obtaining sequencing data previously obtained from a biological sample from a subject;
processing the sequencing data to identify:
a plurality of cell population estimates for a cell of a first type, the plurality of cell population estimates including a first cell population estimate and a second cell population estimate associated respectively with largest and second largest cell population estimates from among the identified plurality of cell population estimates for the cell of the first type; and
features associated with the plurality of cell population estimates, the features including:
a first feature indicative of a size of the first cell population estimate; and
a second feature indicative of a ratio between sizes of the first cell population estimate and the second cell population estimate; and
determining, using the features and a trained machine learning model, whether the first cell population estimate includes malignant cells of the first type.

2. The method of claim 1, wherein processing the sequencing data to identify the plurality of cell population estimates comprises:
obtaining an initial estimate of cell populations; and
generating the plurality of cell population estimates based on the initial estimate, wherein the initial estimate is different from the plurality of cell population estimates.

3. The method of claim 2, wherein the sequencing data comprises a plurality of sequence reads and wherein obtaining the initial estimate of cell populations further comprises grouping sequence reads into groups based on similarity among sequence reads in the plurality of sequence reads.

4. The method of claim 2, wherein the initial estimate of cell populations comprises multiple initial cell population estimates; and
wherein obtaining the initial estimate of cell populations comprises obtaining, for each particular initial cell population estimate of at least some of the multiple initial cell population estimates:
information indicative of a type of receptor chain associated with the particular initial cell population estimate; and
sequence reads associated with the particular initial cell population estimate.

5. The method of claim 4, wherein generating the plurality of cell population estimates further comprises clustering sequence reads associated with at least some of the multiple initial cell population estimates.

6. The method of claim 2, further comprising determining a size of each cell population estimate of the plurality of cell population estimates based on a number of sequence reads associated with each particular initial cell population estimate.

7. The method of claim 4, wherein the receptor chain includes an immunoglobulin heavy chain (IgH) or an immunoglobulin light chain, wherein the immunoglobulin light chain includes at least one of a kappa light chain or a lambda light chain.

8. The method of claim 7, wherein the plurality of cell population estimates comprises a first set of cell population estimates generated for IgH, a second set of cell population estimates generated for IgK, and a third set of cell population estimates generated for IgL.

9. The method of claim 8, wherein the first set includes the first cell population estimate and the second cell population estimate, the second set includes a third cell population estimate and a fourth cell population estimate associated respectively with largest and second largest cell population estimates from among the second set, and the third set includes a fourth cell population estimate and a fifth cell population estimate associated respectively with largest and second largest cell population estimates from among the third set.

10. The method of claim 9, further comprising:
processing the sequencing data to identify:
features associated with the second set of cell population estimates, the features including:
a third feature indicative of a size of the third cell population estimate; and
a fourth feature indicative of a ratio between sizes of the third cell population estimate and the fourth cell population estimate; and
determining, using the features and the trained machine learning model, whether the third cell population estimate includes malignant cells of the first type.

11. The method of claim 10, further comprising:
processing the sequencing data to identify:
features associated with the third set of cell population estimates, the features including:
a fifth feature indicative of a size of the third cell population estimate; and
a sixth feature indicative of a ratio between sizes of the fifth cell population estimate and the sixth cell population estimate; and
determining, using the features and the trained machine learning model, whether the fifth cell population estimate includes malignant cells of the first type.

12. The method of claim 11, further comprising:
obtaining coverages of the second and third sets of cell population estimates; and
determining, based on the coverages and the third and fifth features, whether to output a first result of determining whether the third cell population estimate includes malignant cells of the first type, a second result of determining whether the fifth cell population estimate includes malignant cells of the first type, or neither the first nor the second result.

13. The method of claim 1, wherein the sequencing data comprises RNA sequencing data.

14. The method of claim 1, wherein the sequencing data comprises raw DNA sequencing data, raw RNA sequencing data, DNA exome sequencing data, DNA genome sequencing data, gene sequencing data, bias-corrected gene sequencing data, any sequencing data comprising data obtained from a sequencing platform, or any sequencing data derived from data obtained from a sequencing platform.

15. The method of claim 1, wherein the trained machine learning model is one of a Naïve Bayes classifier, a support vector machine classifier (SVM), a random forest classifier, or an Adaboost classifier.

16. The method of claim 2, further comprising:
generating a graphical user interface (GUI) including a visualization indicating a result of processing the sequencing data, the visualization comprising a plurality of nodes including a first set of nodes, the first set of nodes representing a cell population estimate of the plurality of cell population estimates, wherein each node included in the first set of nodes represents a respective initial cell population estimate of the initial estimate of cell populations.

17. The method of claim 16, wherein the first set of nodes includes a first node representing a first initial cell population estimate of the initial estimate of cell populations and a second node representing a second initial cell population estimate of the initial estimate of cell populations, wherein the first node is connected to the second node by an edge.

18. The method of claim 17, wherein a visual characteristic associated with at least some of the nodes in the first set of nodes is indicative of a characteristic of the first cell population estimate.

19. A system, comprising:
at least one computer hardware processor; and
at least one non-transitory computer-readable storage medium storing processor-executable instructions that, when executed by the at least one computer hardware processor, cause the at least one computer hardware processor to perform:
obtaining sequencing data previously obtained from a biological sample from a subject;
processing the sequencing data to identify:
a plurality of cell population estimates of a cell of a first type, the plurality of cell population estimates including a first cell population estimate and a second cell population estimate associated respectively with largest and second largest cell population estimates from among the identified plurality of cell population estimates for the cell of the first type; and
features associated with the plurality of cell population estimates, the features including:
a first feature indicative of a size of the first cell population estimate; and
a second feature indicative of a ratio between sizes of the first cell population estimate and the second cell population estimate; and
determining, using the features and a trained machine learning model, whether the first cell population estimate includes malignant cells of the first type.

20. At least one non-transitory computer-readable storage medium storing processor-executable instructions that, when executed by at least one computer hardware processor, cause the at least one computer hardware processor to perform:
obtaining sequencing data previously obtained from a biological sample from a subject;
processing the sequencing data to identify:
a plurality of cell population estimates of a cell of a first type, the plurality of cell population estimates including a first cell population estimate and a second cell population estimate associated respectively with largest and second largest cell population estimates from among the identified plurality of cell population estimates for the cell of the first type; and
features associated with the plurality of cell population estimates, the features including:
a first feature indicative of a size of the first cell population estimate; and
a second feature indicative of a ratio between sizes of the first cell population estimate and the second cell population estimate; and
determining, using the features and a trained machine learning model, whether the first cell population estimate includes malignant cells of the first type.


SEQUENCE LISTING (22 sequences)

SEQ ID NO: 1 - protein, 13 aa, Artificial Sequence
  Synthetic
CAASGGSNYKLTF

SEQ ID NO: 2 - protein, 13 aa, Artificial Sequence
  Synthetic
CGAGGTSYGKLTF

SEQ ID NO: 3 - protein, 17 aa, Artificial Sequence
  Synthetic
CARVIETVSNVGLFDIW

SEQ ID NO: 4 - protein, 17 aa, Artificial Sequence
  Synthetic
CARVIETVSKVGLFDIW

SEQ ID NO: 5 - protein, 12 aa, Artificial Sequence
  Synthetic
CARGPINWFDPW

SEQ ID NO: 6 - protein, 11 aa, Artificial Sequence
  Synthetic
CAHSWLPTGDW

SEQ ID NO: 7 - protein, 23 aa, Artificial Sequence
  Synthetic
CARGVGVIEWDMGTITEDGFDPW

SEQ ID NO: 8 - protein, 14 aa, Artificial Sequence
  Synthetic
CARDAPHSSSWQYW

SEQ ID NO: 9 - protein, 24 aa, Artificial Sequence
  Synthetic
CARDRIFGVVIMGMGSYYYAMDVW

SEQ ID NO: 10 - protein, 24 aa, Artificial Sequence
  Synthetic
CARDLRKTGVLFFGESGDYGMDVW

SEQ ID NO: 11 - protein, 19 aa, Artificial Sequence
  Synthetic
CARANEQVVVITNYGMDVW

SEQ ID NO: 12 - DNA, 51 bp, Artificial Sequence
  Synthetic
tgtgcgagagtgatagaaacggtatctaacgtgggcctatttgatatctgg

SEQ ID NO: 13 - DNA, 51 bp, Artificial Sequence
  Synthetic
tgtgcgagagtgatagaaacggtgtctaaggtgggcctgtttgatatctgg

SEQ ID NO: 14 - DNA, 36 bp, Artificial Sequence
  Synthetic
tgtgcgagaggtcccataaattggttcgacccctgg

SEQ ID NO: 15 - DNA, 33 bp, Artificial Sequence
  Synthetic
tgtgcccattcatggctgcctactggggactgg

SEQ ID NO: 16 - DNA, 69 bp, Artificial Sequence
  Synthetic
tgtgcgagaggtgttggtgtcatagagtgggacatgggaacaataacggaagacggtttcgacccctgg

SEQ ID NO: 17 - DNA, 42 bp, Artificial Sequence
  Synthetic
tgtgcgagagacgcaccacatagcagcagttggcagtactgg

SEQ ID NO: 18 - DNA, 72 bp, Artificial Sequence
  Synthetic
tgtgcgagagatcgaatttttggcgtggtaattatggggatgggctcctactactacgctatggacgtctgg

SEQ ID NO: 19 - DNA, 72 bp, Artificial Sequence
  Synthetic
tgtgcgagagatcttcgaaaaacgggagtgctttttttcggggagtcaggtgattacggtatggacgtctgg

SEQ ID NO: 20 - DNA, 57 bp, Artificial Sequence
  Synthetic
tgtgcgagagccaatgagcaggtagtggttattacgaattacggaatggacgtctgg

SEQ ID NO: 21 - protein, 16 aa, Artificial Sequence
  Synthetic
CANGTDPGDPRYFQYW

SEQ ID NO: 22 - protein, 11 aa, Artificial Sequence
  Synthetic
CQQYNGSPLTF